(19) United States
(12) Patent Application Publication
(10) Pub. No.: US 2015/0010474 A1
(43) Pub. Date: Jan. 8, 2015

(54) Compounds And Methods For Selective Imaging And/Or Ablation

(71) Applicant: Auckland Uniservices Limited, Auckland (NL)

(72) Inventors: Robert Forbes Anderson, Auckland (NZ); Jeffery Bruce Smaill, Auckland (NZ); Adam Vorn Patterson, Waiheke Island (NZ); Amir Ashoorzadeh, Manukau (NZ); David Francis Ackerley, Wellington (NZ); Janine Naomi Copp, Nelson (NZ); Alexandra Marie Mowday, Auckland (NZ); Elsie May Williams, Wellington (NZ); Christopher Paul Guise, Auckland (NZ); Cameron Koch, Philadelphia, PA (US); Alex Kachur, Philadelphia, PA (US); William R. Dolbier, JR., Gainsville, FL (US)

(21) Appl. No.: 14/368,261

(22) PCT Filed: Dec. 21, 2012
(86) PCT No.: PCT/NZ2012/000243

§ 371 Date: Jun. 23, 2014

(30) Foreign Application Priority Data
Dec. 23, 2011 (NZ) 597387

(51) Int. Cl. A61K 51/04; A61B 6/03; C07D 403/06; A61K 49/04

(52) U.S. Cl. CPC A61K 51/0453; A61K 49/0442; A61B 6/037; C07D 403/06
USPC 424/1.89; 600/431; 424/9.1; 514/397; 548/255

(57) ABSTRACT

The invention relates generally to compounds and methods for imaging and/or selective ablation of nitroreductase-expressing cells and/or biological agents. More particularly, although not exclusively, the invention provides compounds that are selectively metabolised by bacterial nitroreductases and are substantially insensitive to metabolism under oxic or hypoxic conditions by human nitroreductase enzymes.

The invention relates generally to compounds that have utility in imaging and/or selective ablation of nitroreductase-expressing cells or biological agents. More particularly, although not exclusively, said compounds have use in non-invasive imaging techniques, monitoring of therapeutic cell populations and gene-directed enzyme prodrug therapy.

### BACKGROUND OF THE INVENTION

Selective targeting of cancer tissues can be achieved by tumour-tropic organisms, including certain replication competent viral vectors and bacteria. Such organisms are generally antineoplastic in their own right, and a number are in clinical trials (or clinical use) as novel therapeutic agents. Ideally such agents would be introduced via systemic administration, and would “seek out” cancerous tissues. However, applications to date have been limited owing to an inability to non-invasively image the location of viruses or bacteria in the body post-administration. The self-amplifying nature and uncertain tropism for human tissues has hampered the selection and development of oncolytic viruses and bacteria.

### Non-Invasive Imaging Methods for Biological Vectors

Tissue biopsies and other invasive approaches to imaging tumour-tropic biological vectors cannot be applied to all organs of the body in concert and repeated sampling is rarely clinically feasible. However, the requirement for repeat sample analysis is necessary for dynamic agents that amplify and can redistribute micro-regionally and systemically with time, and mandates a non-invasive methodology that can be applied at regular intervals. This is desirable to allow early intravenous administration of novel vectors in human clinical trials. Of note, animal toxicological models are generally considered to have poor predictive value for human tropic viruses and consequently there is a need to monitor experimental vectors thereby establishing early proof of principle in (preclinical) animal models and in human trials.

Various indirect reporter gene approaches have been tried in an attempt to monitor vector behaviour in living systems including bioluminescence, fluorescence and secreted plasma markers, none of which are considered clinically viable for various reasons including signal attenuation or lack of spatial information.

Positron Emission Tomography (PET) technology is increasingly being applied to the area of therapy development and is the most attractive method for non-invasive and comprehensive measurement of whole body vector distribution. Multiple sampling from the same patient is also possible. PET is safe, accurate and results are reproducible. It also has extremely high sensitivity to imaging probe molecules and is ideal for monitoring cellular or molecular events early in the course of the disease, during therapy, and for evaluating disease recurrence.

PET-based vector imaging has been achieved in preclinical studies for the reporter gene Herpes simplex virus thymidine kinase (HSV-tk) (Bennett et al, 2001, Nat Med 7 (7): 859-863; Gambhir et al, 2000, Proc Natl Acad Sci USA 97 (6): 2785-2790; Soghomonyan et al, 2005, Cancer Gene Ther 12 (1): 101-108) and proof of principle studies are underway with newly designed HSV-tk PET probes (Hackman et al, 2002, Molec Imag 1 (1): 36-42; Jacobs et al, 2001, Cancer Res 61 (7): 2983-2995; Min et al, 2003, Eur J Nuc Med Mol Imaging 30 (11): 1547-1560; Miyagawa et al, 2008, J Nucl Med 49 (4): 637-648) including FHBG (9-(4-[18F]fluoro-3 hydroxymethylbutyl)guanine). However, it has been demonstrated that tumour retention of ¹⁸F-FHBG, monitored via PET, was unsuccessful in predicting HSV-1tk virus load due to tumour release of soluble phosphorylated ¹⁸F-FHBG following tumour cell oncolysis (Kuruppu et al, 2007, Cancer Res 67 (7): 3295-3300). In addition, imaging is hampered using current probes by excessive background signal and a lack of homogenous distribution throughout the body. Other disadvantages to known systems include laborious synthesis of the probes, that the probes can themselves be toxic and easy degradation of probe molecules in the blood, limiting the ability for systemic administration.

### PET Imaging of Tumour Hypoxia Using 2-Nitroimidazoles

Nitroheterocyclic and nitroaromatic compounds of the appropriate electron affinity are known to be capable of being metabolised by human one-electron reductases to form a nitro radical anion that can act as a direct oxygen sensor in cells. In the presence of oxygen this intermediate is rapidly back-oxidised to the parent nitroheterocyclic or nitroaromatic compound in a futile redox cycle resulting in no net overall metabolism. In the absence of oxygen, further reduction of the nitro radical anion can take place to result in the irreversible formation of nitroso and hydroxylamine species (see reaction schema below). These 2-electron and 4-electron reduction intermediates respectively, are capable of covalently reacting with cellular macromolecules, providing cellular retention of the reduction metabolite. 2-Nitroimidazole compounds are known to be of the appropriate electron affinity for human metabolism selectively under hypoxia, such that when these derivatives are radiolabelled (for example with ¹⁸F) the retention of the radiotracer can be used for PET imaging of tumour hypoxia.

The below figure uses [¹⁸F]-EF5 as a specific example of the PET imaging of tumour hypoxia.

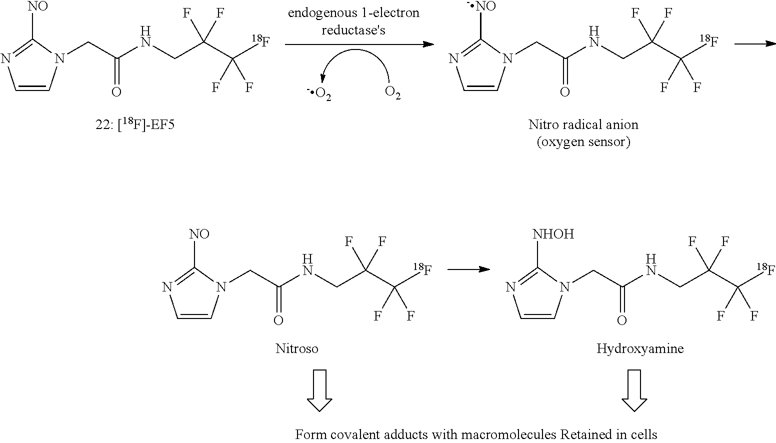

Examples of mesylate, tosylate and alkene radiolabelling precursors for the preparation of known ¹⁸F-labelled 2-nitroimidazole PET imaging agents for the detection of human tumour hypoxia

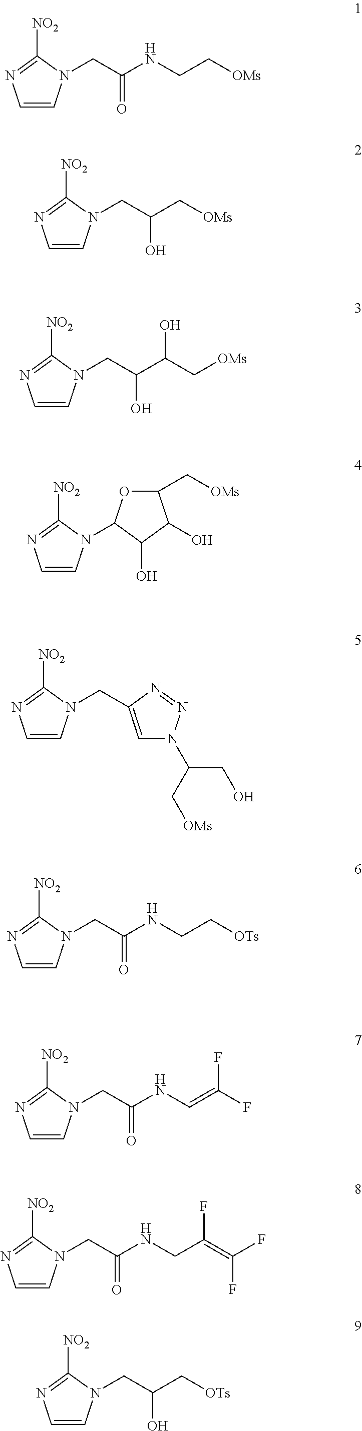

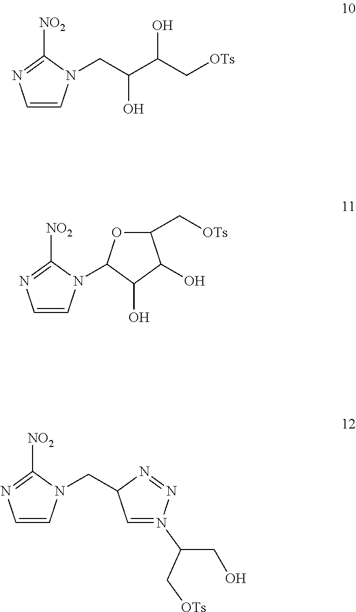

Non-radiolabelled (cold) examples of known 2-nitroimidazole PET imaging agents.

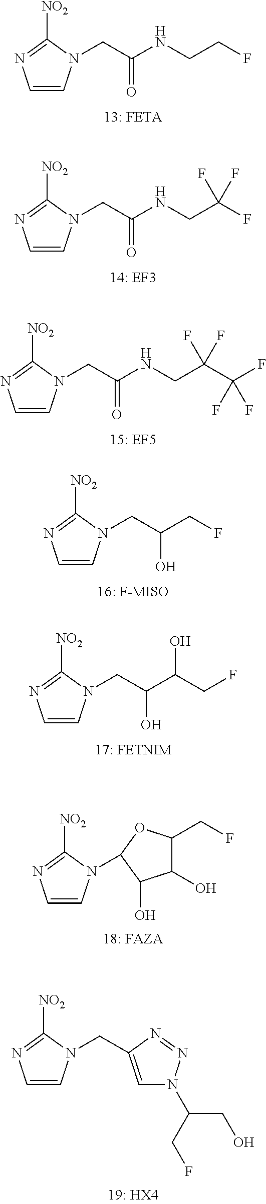

Known ¹⁸F-labelled 2-nitroimidazole PET imaging agents for the detection of human tumour hypoxia

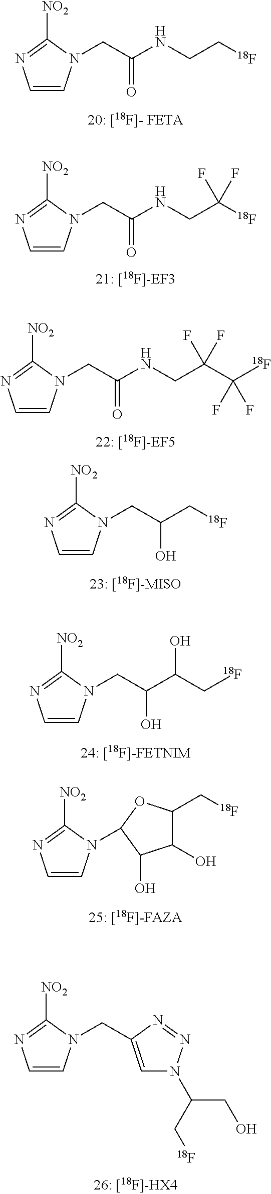

### Use of Bacterial Nitroreductases as Reporter Genes for Imaging

Bacterial nitroreductases (NTRs) can catalyse the reduction of certain nitroheterocyclic/nitrocarbocyclic/nitroaromatic molecules. Limited studies have been conducted on their utility as enzymes for reporter gene systems. Available publications and patents relating to imaging are restricted to the use of fluorescent probe substrates with minimal clinical utility. For example, the non-fluorescent compound 6-chloro-9-nitro-5H-benzo[a]phenoxazin-5-one (C-22220, CNOB) has been described as a fluorogenic probe for detection of nitroreductase activity (Molecular Probes Handbook, Ed. Richard P. Haugland, 10^{th} Edition, 2005, p 535). Escherichia coli NfsB can metabolise CNOB to a fluorescent aminophenoxazine (Ex/Em 617/625 nm) and CNOB has been used for the detection of E. coli nfsB expression in tumour bearing nude mice injected with E. coli NfsB-expressing Clostridia sporogenes spores (Liu et al, 2008, Cancer Res 68 (19): 7995-8003).

The non-fluorescent 6-nitroquinoline has been described as a fluorogenic probe for the detection of E. coli nfsB expression in cell culture monolayers (Singleton et al, 2007, Cancer Gene Ther 14(12): 953-967). In a further example, CytoCy5 is a cell-entrapped red fluorescent probe for E. coli NfsB with putative utility in vivo (U.S. Pat. No. 7,579,140). These systems are inadequate as nitroreductase-based reporter gene systems for clinical applications due to problems including signal attenuation and lack of spatial information.

Thus it is desirable to provide alternative non-invasive nitroreductase-based reporter gene imaging technologies that preferably allow for rapid, reproducible and quantitative imaging and/or that enable the monitoring of gene/vector and amplitude in the same patient or animal over time. Additionally, there would be an advantage in providing imaging technologies to monitor the spatial and temporal distribution of nitroreductase-based vector systems with time in a manner that is predictive of normal tissue toxicity and antitumour efficacy.

### Gene-Directed Enzyme Prodrug Therapy (GDEPT)

Gene-directed enzyme prodrug therapy (GDEPT) is a gene therapy strategy in which a therapeutic gene encodes an exogenous enzyme that will convert an administered non-toxic prodrug into an active cytotoxic derivative. GDEPT is made up of three components; the prodrug to be activated, the prodrug activating enzyme, and the delivery vector for the corresponding gene. Preferential activation of the prodrug in transduced tumour cells generates high intra-tumoural drug (activated prodrug metabolite) concentrations and therefore increases the therapeutic index of the drug.

It would be preferable to be able to utilise a single enzyme or gene product to enable both imaging and prodrug activation as imaging may directly predict the location and magnitude of prodrug activation, providing critical safety information prior to introduction of a conditionally cytotoxic therapy component.

Selectivity for tumour (over normal) tissues is predicated on the use of a biological vector, such as an oncolytic virus, that has been targeted to the tumour tissues. Therapy that utilises viral delivery vehicles is also known as virus-directed enzyme prodrug therapy (VDEPT). Alternatively, use of bacterial vectors tropic for tumour tissues, such as Clostridia sp., Salmonella sp. or Bifidobacter sp. is commonly termed bacterial-directed enzyme prodrug therapy (BDEPT), or in certain specific cases CDEPT (for Clostridia-directed enzyme prodrug therapy). These are all vector specific variants of GDEPT and are considered to be covered by this common acronym. An additional term, ADEPT, refers to antibody-directed enzyme prodrug therapy and encompasses the use of epitope-specific antibodies to guide systemically administered antibody-enzyme fusions to tumour sites in order to target prodrug activation.

The limited activity of GDEPT systems has led to the evaluation of the E. coli nitroreductase NfsB in combination with CB1954 (5-aziridinyl-2,4-dinitrobenzamide) and various other nitroheterocyclic/nitrocarbocyclic/nitroaromatic prodrugs (Denny, 2002, Curr Pharm Des 8 (15): 1349-1361; Searle et al, 2004, Clin Exp Pharmacol Physiol 31 811-816; Singleton et al, 2007, Cancer Gene Ther 14(12): 953-967). The NfsB/CB1954 combination has undergone evaluation in a VDEPT setting with some signs of activity (Palmer et al, 2004, J Clin Oncol 22 (9): 1546-1552). Alternate NTRs, an evolved form of E. coli YieF (Barak et al, Mol Can Ther 5 (1): 97-103) and wild-type E. coli NfsA (Vass et al, 2009, Br J Cancer 100 (12): 1903-1911; Prosser et al, 2010, Biochem Pharmacol 79, 678-687) have been evaluated in combination with CB1954 (and the former also with mitomycin C and CNOB (C-22220) (Thorne et al, 2009, Mol Can Ther 8 (2): 333-341)). Bacillus amyloliquefaciens YwrO and Enterobacter cloacae NR are also known to reduce the prodrug CB1954 (Anlezark et al, 2002, Microbiology 148 (Pt 1): 297-306).

The currently known and studied bacterial nitroreductase enzymes for GDEPT have not been shown to be capable of metabolising known 2-nitroimidazole hypoxia PET imaging agents, suggesting it will not be possible to re-purpose these agents for the non-invasive imaging of nitroreductase-based vector distribution and amplitude in the same patient or animal over time. Indeed should bacterial enzymes capable of metabolising this class of PET agent become available, imaging of tumour hypoxia will likely be a complicating factor in the detecting of the bacterial reporter gene, compromising their utility in this context.

The ability to ablate cells without localised damage to neighbouring tissue (known as single cell ablation) is seen as a valuable safety control for enabling the elimination of a vector in the matrix, cells or tissues should this be deemed necessary. The ability to control viral (VDEPT) or bacterial (BDEPT) infection is an additional biosafety feature and is considered to be a desired design feature in replicating biological vectors. To achieve this, activation of prodrugs that provide reduced, substantially minimal or zero bystander effect is also desirable.

### Detection of Bacterial Nitroreductases

Thus there is a need for nitroheterocyclic/nitroaromatic PET imaging agents that are selectively metabolised by bacterial nitroreductases and therefore retained in cells. Further these agents should be insensitive to metabolism in mammalian cells under either oxic or hypoxic conditions, allowing for optimised signal to noise in the context of the non-invasive imaging of bacterial nitroreductase based biological vectors for gene therapy applications. Further, in their ‘cold’ or non-radiolabelled form at high dose, it would be advantageous if such nitroheterocyclic/nitroaromaticagents through the metabolism of the bacterial nitroreductase should result in ‘single cell ablation’ of the bacterial nitroreductase expressing cell or biological vector with minimal cytotoxicity to neighbouring cells. This desirable feature can allow for the selective eradication of the replicating biological vector, and can be achieved through designing a substantially minimal bystander effect into the reduction metabolites of the nitroheterocyclic/nitroaromatic agents.

It is an object of the invention to meet at least one of the foregoing needs, to overcome or ameliorate at least one of the disadvantages of the prior art, or at least to provide the public with a useful choice.

### SUMMARY OF THE INVENTION

In a first aspect, the invention provides a method of imaging and/or ablation of a bacterial nitroreductase-expressing cell and/or a bacterial nitroreductase-expressing biological agent comprising:
a. introduction of a compound of formula I to a subject; and
b. metabolising the compound with a bacterial nitroreductase expressed by the cell and/or biological agent;

wherein the compound is substantially insensitive to metabolism under oxic or hypoxic conditions in a cell or biological agent that does not express a bacterial nitroreductase; and wherein formula I comprises:

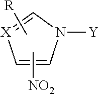

wherein:
a) when X═O, S or C—H,
   R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
   NO₂ is attached at any unsubstituted position; and
   Y comprises a formula selected from the group consisting of formulae IIa to IIg:

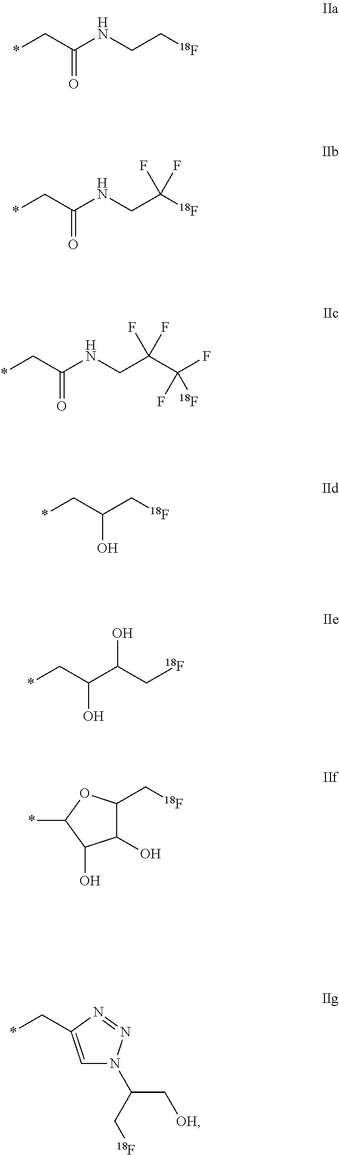

and IIIa to IIIh;

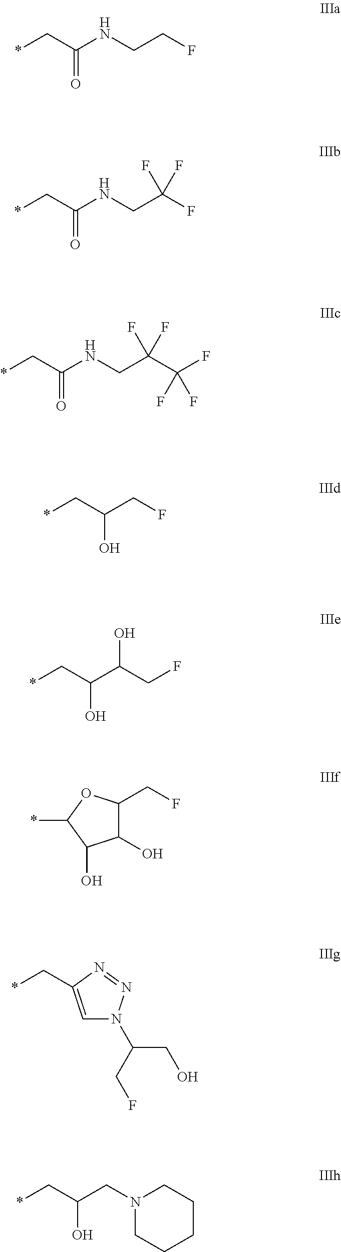

where *=a point of attachment to Formula I; or
b) when X═N,
   R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
   NO₂ is attached at the 4- or 5-position; and
   Y is selected from the group consisting of formulae IIa-g and IIIa-h where *=a point of attachment to Formula I.

In a particular embodiment, the method comprises a method of imaging and Y is selected from groups IIa to IIg. Preferably, the method is a PET or SPECT imaging method.

In a particular embodiment, the method comprises a method of single cell ablation and Y is selected from groups IIIa to IIIh. Preferably, the compound has a minimal bystander effect.

In a particular embodiment, the method comprises a method of imaging, and Y is selected from groups IIIb, IIIc or IIIh, and R is selected from CH₂F or CH₂¹⁸F. Preferably, the compound is recognized and bound by an antibody specific to the compound. Preferably, the method is a method of immunohistochemical imaging.

In a particular embodiment, the method comprises a method of imaging and Y is group IIg. This embodiment has particularly utility as an imaging agent because such compounds in their free unbound form are believed to have the capacity to be quickly removed from the body during and after administration therefore minimizing background radiosignal readily allowing for detection of the bound form.

In a particular embodiment, the method comprises the use of a compound comprising:
a. a radiolabelled compound according to formula 104:

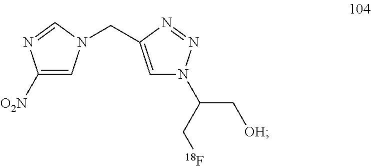 [SMILES: O=[N+]([O-])c1cn(Cc2cn(C(CO)C[18F])nn2)cn1]

and/or
b. a compound according to formula 97:

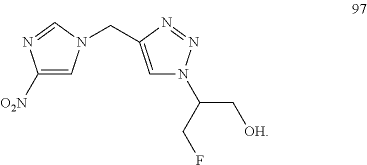 [SMILES: O=[N+]([O-])c1cn(Cc2cn(C(CO)CF)nn2)cn1]

In a particular embodiment, the nitroreductase enzyme is expressed by a wild type or mutant variant of E coli NfsA.

In a second aspect, the invention provides a compound of formula I:

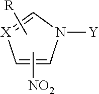

wherein:
1) when X═O, S or C—H,
   R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
   NO₂ is attached at any unsubstituted position; and
   Y comprises a formula selected from the group consisting of formulae IIa to IIg and IIIa to IIIh where *=a point of attachment to Formula I;
   or a precursor thereof; or
2) when X═N,
   R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
   NO₂ is attached at the 4- or 5-position; and
   Y is selected from the group consisting of: formulae IIa-g and IIIa-c and Ille-h where *=a point of attachment to Formula I;
   or a precursor thereof.

In a particular embodiment of the second aspect, the compound is a precursor compound and Y is selected from the group consisting of formulae IVa-g:

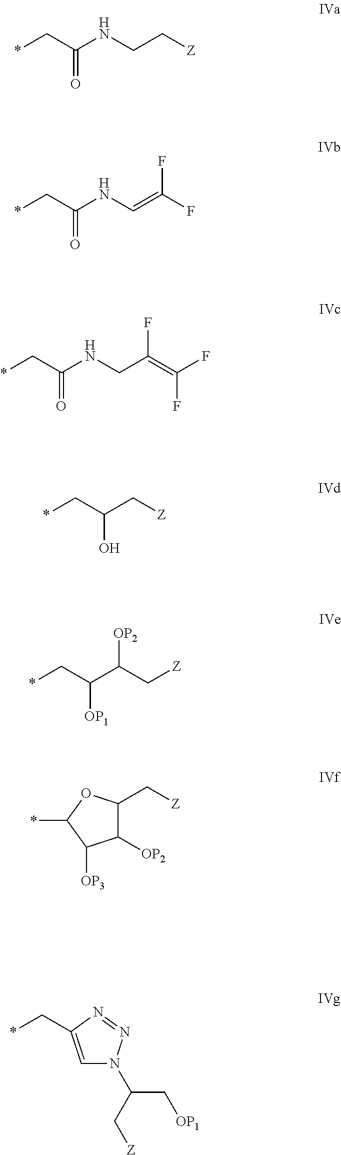

where *=a point of attachment to Formula I and Z═Cl, Br, I, OSO₂CH₃, OTs, ONs, OSO₂CF₃ and P₁ and P₂ can be independently selected from H, CO(C₁-C₆ alkyl), CO^{t}Bu, Si(CH₃)₃, Si(CH₃)₂^{t}Bu, Si(Ph)₂^{t}Bu, CH₂Ph, CH₂C₆H₄OMe, C(Ph)₃ or together may form an acetonide ring.

In a third aspect, the invention provides a compound of formula V:

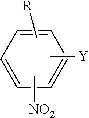

wherein:
R═H, CH₂¹⁸F, CH₂F, CF₃, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
NO₂ is attached at any unsubstituted position; and
Y is selected from the group consisting of formulae IIa-g and IIIa-h as defined in the first aspect, where *=a point of attachment to Formula V;

or a precursor thereof.
In a particular embodiment of the third aspect, the compound is a precursor compound and Y is selected from the group consisting of formula IVa-g as defined in the first aspect, Z ═Cl, Br, I, OSO₂CH₃, OTs, ONs, OSO₂CF₃ and P₁ and P₂ can be independently selected from H, CO(C₁-C₆ alkyl), CO^{t}Bu, Si(CH₃)₃, Si(CH₃)₂^{t}Bu, Si(Ph)₂^{t}Bu, CH₂Ph, CH₂C₆H₄OMe, C(Ph)₃ or together may form an acetonide ring.

In a further aspect, the invention provides a method of imaging and/or ablation of a bacterial nitroreductase-expressing cell and/or a bacterial nitroreductase-expressing biological agent comprising:
a. introduction of a compound as described in the third aspect excluding precursors, to a subject; and
b. metabolising the compound with a bacterial nitroreductase expressed by the cell and/or biological agent;

wherein the compound is substantially insensitive to metabolism under oxic or hypoxic conditions in a cell or biological agent that does not express a bacterial nitroreductase.

In a particular embodiment, the method comprises a method of imaging and Y is selected from groups IIa to IIg. Preferably, the method is a PET or SPECT imaging method.

In a particular embodiment, the method comprises a method of single cell ablation and Y is selected from groups IIIa to IIIh. Preferably, the compound has a minimal bystander effect.

In a particular embodiment, the method comprises a method of imaging, and Y is selected from groups IIIb, IIIc or IIIh, and R is selected from CH₂F or CH₂¹⁸F. Preferably, the compound is recognized and bound by an antibody specific to the compound. Preferably, the method is a method of immunohistochemical imaging.

In a particular embodiment, the method comprises a method of imaging and Y is group IIg. This embodiment has particularly utility as an imaging agent because such compounds in their free unbound form are believed to have the capacity to be quickly removed from the body during and after administration therefore minimizing background radiosignal readily allowing for detection of the bound form.

In a fourth aspect, the invention provides a compound of general formula I wherein:
X═N, O, S or C—H;
R═CH₂¹⁸F or CH₂F;
NO₂ is attached at the 4- or 5-position; and
Y is selected from formulas IIIb, IIIc or IIIh where *=a point of attachment to formula I; or a precursor thereof.

In a particular embodiment of the fourth aspect, the compound is a precursor compound and R═CH₂Z, where Z═Cl, Br, I, OSO₂CH₃, OTs, OSO₂CF₃.

In a fifth aspect, the invention provides a compound of general formula V wherein:
R═CH₂¹⁸F or CH₂F;
NO₂ is attached at any unsubstituted position; and
Y is selected from formulas IIIb, IIIc or IIIh where *=a point of attachment to formula V; or a precursor thereof.

In a particular embodiment of the fifth aspect, the compound is a precursor compound and R ═CH₂Z, where Z═Cl, Br, I, OSO₂CH₃, OTs, OSO₂CF₃.

In a sixth aspect, the invention provides a method of imaging and/or ablation of a bacterial nitroreductase-expressing cell and/or a bacterial nitroreductase-expressing biological agent comprising:
a. introduction of a compound as described in the fourth or fifth aspect excluding precursors, to a subject; and
b. metabolising the compound with a bacterial nitroreductase expressed by the cell and/or biological agent.

In a further aspect, the invention provides a method of treatment or diagnosis of a disease using a compound as defined in any one of the first to the fifth aspects wherein the disease is selected from the group consisting of cancer, Parkinson's disease, Alzheimer's disease, stroke, heart disease, rheumatological diseases and a disease treated by stem-cell transplantation.

[Methods of treatment using any compound (SSC version)] In a further aspect, the invention provides the use of a compound as defined in any one of the first to the fifth aspects in the manufacture of a medicament for the treatment of a disease selected from the group consisting of cancer, Parkinson's disease, Alzheimer's disease, stroke, heart disease, rheumatological diseases and a disease treated by stem-cell transplantation.

In a further aspect, the invention provides a compound as defined in any one of the first to the fifth aspects for use in the treatment of a disease selected from the group consisting of cancer, Parkinson's disease, Alzheimer's disease, stroke, heart disease, rheumatological diseases and a disease treated by stem-cell transplantation.

In a further aspect, the invention provides a composition comprising a compound as defined in any one of the first to the fifth aspects and a pharmaceutically acceptable diluent, excipient, carrier or adjuvant.

In a further aspect, the invention provides a kit for evaluation of in vivo distribution of a nitroreductase-expressing cell and/or biological agent comprising a compound as defined in any one of the first to the fifth aspects of the invention.

In a further aspect, the invention provides a kit comprising a one or more of:
a. a radiolabelled compound according to formula 104:

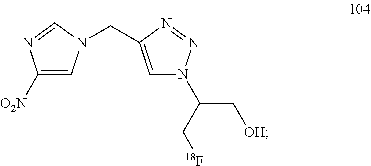 [SMILES: O=[N+]([O-])c1cn(Cc2cn(C(CO)C[18F])nn2)cn1]

b. a precursor compound according to formula 367:

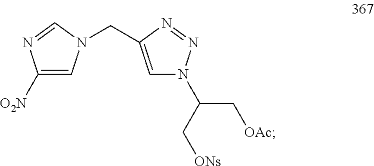

and/or
c. a compound according to formula 97:

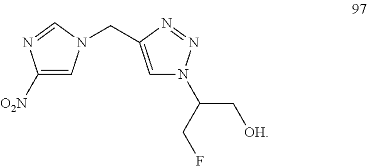 [SMILES: O=[N+]([O-])c1cn(Cc2cn(C(CO)CF)nn2)cn1]

In a particular embodiment, the kit is used in conjunction with a nitroreductase enzyme expressed by a wild type or mutant variant of E coli NfsA.

In a further aspect, the invention provides a kit for the control of a cell and/or a biological agent comprising a compound as defined in any one of the first to the fifth aspects of the invention.

In a further aspect, the invention provides a method of synthesis of a compound as defined in any one of the first to the fifth aspects of the invention.

In a particular embodiment, the method of synthesis comprises a method as described hereinafter.

In a particular embodiment, the method comprises a) a fluoride displacement of a mesylate, tosylate or nosylate followed by in situ deprotection of any protecting groups where necessary or b) a fluorine gas addition to a double bond or c) amide coupling of fluorinated amine intermediates with their acid counterparts to provide “cold” fluorine containing compounds or d) click coupling of azide intermediates with alkynes to provide triazole derivatives.

In a particular embodiment, the compound comprises compound 67 and 93 and the method comprises a Swern oxidation and an alkylation, respectively, as described below:

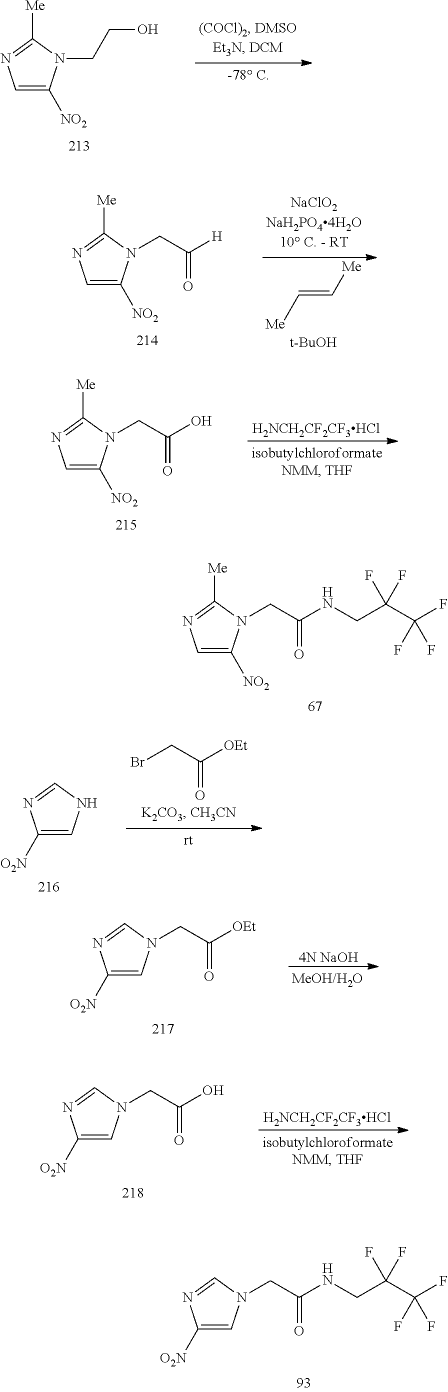

In a further aspect, the invention provides a method of synthesising a non-precursor compound as defined in any one of the first to the fifth aspects using a precursor compound as defined in any one of the first to the fifth aspects.

In a particular embodiment, the method comprises a) a fluoride displacement of a mesylate, tosylate or nosylate followed by in situ deprotection of any protecting groups where necessary or b) a fluorine gas addition to a double bond or c) amide coupling of fluorinated amine intermediates with their acid counterparts to provide “cold” fluorine containing compounds or d) click coupling of azide intermediates with alkynes to provide triazole derivatives.

In a further aspect, the invention provides a method of selecting a nitroheterocyclic or nitroaromatic compound for use in a method of imaging and/or ablation of a bacterial nitroreductase-expressing cell and/or a bacterial nitroreductase-expressing biological agent, the method comprising:
a. under both oxic and hypoxic conditions, separately measuring the sensitivity of the compound to metabolism by a human nitroreductase enzyme and a bacterial nitroreductase enzyme; and
b. selecting the compound if it is
   i. substantially insensitive to metabolism by a human nitroreductase enzyme; and
   ii. metabolised by a bacterial nitroreductase enzyme.

In a particular embodiment, the sensitivity to a human nitroreductase enzyme is measured by determining the one-electron reduction potential of the compound and the compound is selected if the one-electron potential is too low to accept electrons from human enzymes. Preferably, the one-electron reduction potential of the compounds selected is less than approximately −490 mV. Compounds selected by this method have utility in any of the methods of treatment described herein

Further aspects of the invention, which should be considered in all its novel aspects, will become apparent to those skilled in the art upon reading of the following description which provides at least one example of a practical application of the invention.

### DESCRIPTION OF THE FIGURES

Embodiments of the invention will now be described, by way of example only, with reference to the accompanying drawings in which:

FIG. 1 illustrates the family relationships of the 58 nitroreductase (NTR) candidates in the E. coli NTR over-expression library, derived from 13 bacterial enzyme families.

FIG. 2 illustrates the metabolism of compound 67 by members of the 58-membered NTR over-expression library as measured by (A) Growth Inhibition assay and (B) SOS assay.

FIG. 2.1 illustrates the metabolism of compound 93 by members of the 58-membered NTR over-expression library as measured by Growth Inhibition assay.

FIG. 2.2 illustrates the metabolism of compound 97 by members of the 58-membered NTR over-expression library as measured by Growth Inhibition assay.

FIG. 2.3 illustrates the IC₅₀ of compound 67 for selected NTR library strains.

FIG. 2.4 illustrates the IC₅₀ of compound 93 for selected NTR library strains.

FIG. 2.5 illustrates the IC₅₀ of compound 97 for selected NTR library strains.

FIG. 3 illustrates the results of flow cytometry analysis of HCT-116 cells stably expressing E. coli NfsA relative to HCT-116 wild-type cells after in vitro exposure to 200 of compounds 15, 93 and 67 for 2 hours.

FIG. 4 illustrates the results of a second independent flow cytometry analysis of compound 15 and 93 metabolism and binding in wild-type HCT-116 cells, HCT-116 cells stably over-expressing cytochrome P450 reductase (CYPOR), a human one-electron reductase known to metabolise nitroheterocyclic and nitroaromatic compounds, or HCT-116 cells stably expressing the bacterial nitroreductase E. coli NfsA.

FIG. 5 illustrates the results of flow cytometry analysis of HCT-116 cells stably over-expressing cytochrome P450 reductase (CYPOR), a human one-electron reductase known to metabolise nitroheterocyclic and nitroaromatic compounds. 1×10⁶HCT-116-CYPOR cells were seeded in 6 well plates in aerobic, anoxic and 0.2% oxygen conditions designed to replicate the lower limit of pathological hypoxia observed in human tumours.

FIG. 6 illustrates the results of flow cytometry analysis of compound 15 and 93 metabolism and binding in wild-type HCT-116 cells and HCT-116 cells stably over-expressing cytochrome P450 reductase (CYPOR), a human one-electron reductase known to metabolise nitroheterocyclic and nitroaromatic compounds.

FIG. 7 illustrates immunohistochemical detection of ‘cold’ EF5 (compound 15) binding in human tumour xenografts harbouring 0% or 25% HCT-116 NfsA-expressing cells.

FIG. 8 illustrates the in vivo binding of compounds 15, 93 and 67 in the human lung tumour xenograft NCI-H1299 harbouring approximately 5% NfsA-positive cells.

FIG. 9 illustrates the absence of hypoxic dependent binding of compound 67 in the human solid tumour xenograft HCT116 relative to compound 15 whilst including hypoxia co-staining by pimonidazole (Hypoxyprobe™) as an internal reference (positive control).

FIG. 10 illustrates the absence of hypoxic-dependent binding of compound 67 and compound 93 by fluorescent immune-histochemistry in the human solid tumour xenograft NCI-H1299, with reference to hypoxia staining by compound 15 and pimonidazole (Hypoxyprobe™) as internal standards (positive controls).

FIG. 11 illustrates the absence of hypoxic-dependent binding of compound 67 and compound 93 by flow cytometry in the human solid tumour xenograft NCI-H1299, with reference to hypoxia staining by compound 15 and pimonidazole (Hypoxyprobe™) as internal standards (positive controls).

### DETAILED DESCRIPTION OF THE INVENTION

### Definitions

EF3 also called trifluoroetanidazole, also called 2-(2-nitro-1H-imidazol-1-yl)-N-(2,2,2-trifluoroethyl)acetamide

EF5 also called pentafluoroetanidazole, also called 2-(2-nitro-1H-imidazol-1-yl)-N-(2,2,3,3,3-pentafluoropropyl)acetamide

F-MISO also called fluoromisonidazole, also called 1-fluoro-3-(2-nitro-1H-imidazol-1-yl)propan-2-ol

Metronidazole also called 2-(2-methyl-5-nitro-1H-imidazol-1-yl)ethanol

“Mesylate”—An ester of methanesulfonic acid (CH₃SO₃H). A group of organic compounds that share a common functional group with the general structure CH₃SO₂O—R, abbreviated as MsO-R, where R is an organic substituent. Mesylate is considered an excellent leaving group in nucleophilc substitution reactions. Also called a mesyl group.

“Tosylate”—An ester of p-toluenesulfonic acid (CH₃C₆H₄SO₃H). A group of organic compounds that share a common functional group with the general structure CH₃C₆H₄SO₂O—R, abbreviated as TsO-R, where R is an organic substituent. Tosylate is considered an excellent leaving group in nucleophilc substitution reactions. Also called a tosyl group.

“Nosylate”—An ester of 2-nitrobenzenesulfonic acid (2-NO₂C₆H₄SO₃H) or 4-nitrobenzenesulfonic acid (4-NO₂C₆H₄SO₃H). A group of organic compounds that share a common functional group with the general structure NO₂C₆H₄SO₂O—R, abbreviated as NsO-R, where R is an organic substituent. Nosylate is considered an excellent leaving group in nucleophilc substitution reactions. Also called a nosyl group.

“Nitroreductase” or “NTR”—an enzyme that catalyses the reduction of a nitro functional group (—NO₂) or quinine functional group. As referred to herein, “nitroreductase” or “NTR” is to be taken to mean a bacterial nitroreductase, i.e. a nitroreductase of bacterial origin.

“Prodrug”—An inactive compound that is converted to a reactive cytotoxic metabolite once activated that may have an endogenous or exogenous effect (see bystander effect). Preferably activation occurs within target cells or within the local microenvironment by reduction or selective action of a target-cell-specific enzyme. Prodrugs may also be activated by differences in pH/oxygenation between target and non-target tissue. Prodrugs include precursors to anti-parasitic agents. As well as being activated in a cell and/or biological agent, it is also contemplated that the prodrug is activated in a matrix.

“Matrix”—this term refers to the material that may support or contain a cell and/or biological agent. The term includes a tissue or a growth medium and the matrix may be found in vivo or in vitro.

“Ablation” is to be considered in its broadest context and as well meaning the complete ceasing of the function of the target being ablated, is also intended to encompass any degree of suppression of the function of the target where the target includes but is not limited to a cell or a biological agent.

“Imaging probe” or “probe”—a compound or agent that is labelled in such a way that it, or it's derivative can be detected by an imaging technique. The process may be used to detect, identify or obtain information about another substance in a sample or tissue. Imaging probes are often labelled using radioactive labels for use in non-invasive imaging (bio-detection) or radioimaging. In particular embodiments, radiolabelled imaging probes (or “radiotracers”) may be used to label particular tissues or cells for detection using Positron Emission Tomography (PET), micro-Positron Emission Tomography (micro-PET) or Single Photon Emission Tomography (SPECT). The labels for such imaging probes may comprise a positron-emitting nuclide such as ¹⁵O, ¹³N, ¹¹C, ¹²⁴I, ⁷⁶Br and ¹⁸F or a gamma-emitting nuclide such as ^{99m}Tc, ⁶⁷Ga, ¹¹¹In and ¹²³I. Imaging probes also include “cold” versions of a radiolabelled imaging probe labelled with a non-radioactive isotope (e.g. ¹⁹F). Such “cold” imaging probes have use in immunohistochemical staining techniques as they may have a particular structural conformation that can act as a substrate for antibodies detectable by Fluorescence-activated cell sorting (FACS) which is a specialized type of flow cytometry.

“Activation” or “metabolism” with reference to the compounds of use in the invention refers to the catalytic reduction process that the compound may undergo following contact with an enzyme. The compound may be activated/metabolised to yield alternative compounds that may have beneficial activity for imaging or therapeutic applications. The metabolites may also be retained by a cell, matrix and/or biological agent which can enable the temporal analysis of probe/prodrug distribution. Metabolism of a particular compound by a nitroreductase enzyme can be measured by incubating the compound and the purified recombinant enzyme in the presence of NADPH co-factor and following the loss of such co-factor by UV/Vis spectroscopy. Consumption of co-factor directly indicates enzymatic metabolism of the compound. Metabolism can also be identified by comparing the cytotoxicity or growth inhibition of test compounds in mammalian or bacterial cell lines that are engineered to over-express the enzyme, compared to the non-expressing control cell lines. Increased anti-proliferative activity or cytotoxicity of the compound selectively in the enzyme-expressing cell line indicates metabolism of the compound by the enzyme to metabolites with increased anti-proliferative or cytotoxic activity. Further, metabolism can be identified by incubating a compound in the presence of mammalian or bacterial cell lines that are engineered to over-express the enzyme, compared to the non-expressing control cell line followed by detection of cellular binding of the metabolites using immunohistochemistry. Increased metabolite binding in enzyme-expressing cells relative to the control cell line indicates enzymatic metabolism of the compound. Immunohistochemical assays such as this can be performed in vitro or following administration of compounds to tumour-bearing animals and isolation of the tumour and cross-sectioning ex vivo. When used in relation to immunohistochemical imaging, metabolism may also be taken to mean that the compound is recognized and bound by an antibody specific to the compound.

“Substantially insensitive to metabolism” when used in reference to oxic or hypoxic conditions is intended to refer to a compound that exhibits a very low or substantially zero degree of metabolism by a human nitroreductase enzyme when compared to a compound that is readily metabolised by human enzymes under hypoxia such as EF5. In a particular embodiment, the degree of metabolism of a compound that is substantially insensitive to metabolism is between 5 and 100 times less, preferably between 9 and 67 times less, than the metabolism of EF5 under substantially identical conditions. This lack of metabolism may be determined by the lack of detection of metabolite binding in control wild type (bacterial nitroreductase enzyme non-expressing) cells following incubation with the test compound. In a particular embodiment, detection is by immunohistochemical imaging of the bound metabolite adducts. In a particular embodiment, the sensitivity is measured by determining the one-electron reduction potential of the compound. The compound is determined to be substantially insensitive to metabolism if the one-electron reduction potential is too low to accept electrons from human enzymes. In a particular embodiment, substantially insensitive to metabolism indicates that the compound has a one-electron reduction potential of less than approximately −490 mV.

“Oxic conditions” refers to ambient atmospheric oxygen tension of approximately 4-21%.

“Hypoxic conditions” refers to oxygen tensions below approximately 1% (10,000 parts per million oxygen; 7.6 mmHg).

While the specification refers to compounds being “substantially insensitive to metabolism under oxic or hypoxic conditions” and a definition of hypoxic and oxic is provided, when used in reference to the sensitivity of a compound to metabolism by a bacterial NTR, this phrase indicates that the sensitivity of the compound is substantially independent of the oxygen status of the cell/biological agent.

“Precursor” refers to an intermediate compound that typically possesses a good leaving group such as a mesylate, tosylate or nosylate that can undergo reaction with a substituent group. In a particular embodiment, the substituent group is a radionucleotide such as 18-F-fluoride to provide a radiotracer or compound for PET or SPECT imaging purposes.

“Nitroimidazole or a derivative thereof”—this term includes substituted and unsubstituted nitroimidazole compounds including substituted and unsubstituted 2-nitroimidazole, 4-nitroimidazole, and 5-nitroimidazole compounds.

“Cell” refers to a biological sub-unit that is specialized in carrying out a particular function or functions. For the purposes of the invention as defined herein, the term “cell” also encompasses the medium in which the cell is found. For example this may mean a hypoxic region of a tumour or the cell matrix which supports the cell in vivo or in vitro.

“Biological agent” encompasses any biological unit (except cells as defined above) on which an activated prodrug may act and that has the capacity to express or deliver a nitroreductase enzyme. This term includes, but is not limited to vectors (particularly plasmid vectors), viruses (particularly adenovirsues, vaccinia virus, measles virus, picornaviruses), bacteria (particularly Clostridium sp. and Salmonella sp.), liposomes, nanoparticles, and antibodies. The term “nitroreductase-expressing biological agent” encompasses a biological agent that expresses a nitroreductase as well as a biological agent that does not directly express the nitroreductase but delivers it to a target tissue (for example in ADEPT). The NTR expressing cell/biological agent may be delivered according to any methods known in the art. Particularly methods described in the background section including VDEPT, BDEPT, CDEPT or GDEPT.

“Endogenous”—Naturally occurring, originating or produced within an organism, tissue, or cell. For example endogenous enzymes in a mammal are enzymes that are naturally present in mammalian cells.

“Exogenous”—Originating or produced outside of an organism, tissue, or cell. For example exogenous enzymes in a mammal are foreign enzymes that do not occur in mammalian cells. For example bacterial enzymes that may have been introduced through genetic manipulations.

“Bystander effect”—this effect is triggered by treatment of a target cell with a prodrug and refers to the secondary ablation effect on cells or tissues in the local microenvironment to the target cell/biological agent. Without wishing to be bound by theory, the bystander effect is believed to be caused by the diffusion of cytotoxic prodrug metabolites (activated prodrugs) from the site of production to affect unmodified cells exogenous to the target cell.

“Vector” encompasses any vehicle for the delivery of an enzyme or gene to a target. Examples of vectors include includes viruses, bacteria, plasmids, liposomes, nanoparticles, antibodies, human multipotent marrow stromal cells or genetic vectors but the vector may also be a cell, for example a stem cell.

“Treatment” is to be considered in its broadest context. The term does not necessarily imply that a subject is treated until total recovery. Accordingly, “treatment” broadly includes, for example, the prevention, amelioration or management of the disease, one or more symptoms of the disease, or the severity of one or more symptoms. It also includes the preventing or otherwise reducing the risk of developing secondary complications. development is completely prevented, and include delay of disease development.

### INVENTION DISCLOSURE

The following is a description of the present invention, including preferred embodiments thereof, given in general terms. The invention is further elucidated from the disclosure given under the heading “Examples” herein below, which provides experimental data supporting the invention, specific examples of various aspects of the invention, and means of performing the invention.

The invention provides a method of imaging and/or ablation of a bacterial nitroreductase-expressing cell and/or a bacterial nitroreductase-expressing biological agent comprising:
a. introduction of a compound of formula I (as defined above) to a subject; and
b. metabolising the compound with a bacterial nitroreductase expressed by the cell and/or biological agent;

wherein the compound is substantially insensitive to metabolism under oxic or hypoxic conditions in a cell or biological agent that does not express a bacterial nitroreductase.

### Imaging Using Compounds of the Invention

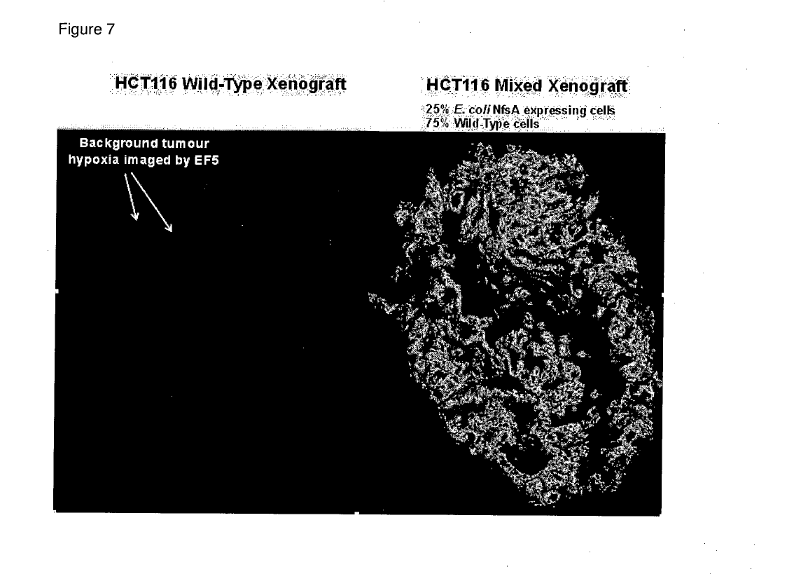

Among other uses, bacterial NTR-expressing cells or biological agents are introduced to a subject and used to image and/or treat tumours. Known imaging and prodrug combinations that are sensitive to metabolism by an NTR may be used to determine the distribution and amplitude of the NTR-expressing cell/biological agent. However, hypoxic regions of tumour tissue result in known imaging compounds being metabolised leading to undesirable background signal when imaging these NTR-expressing entities. Example 8 (FIGS. 7, 9, 10, 11) illustrates this undesirable background signal caused by metabolism and binding of EF5 and pimonidazole in the hypoxic regions of the tumour.

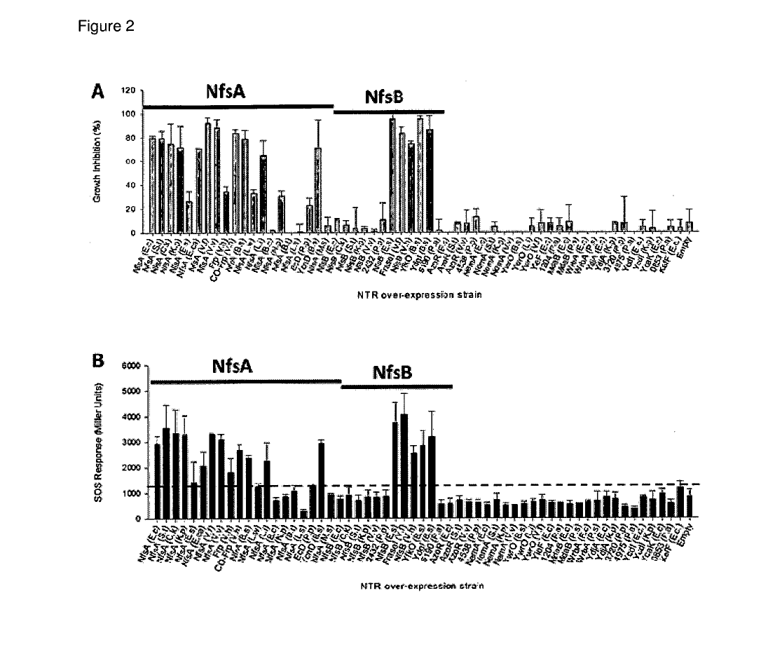

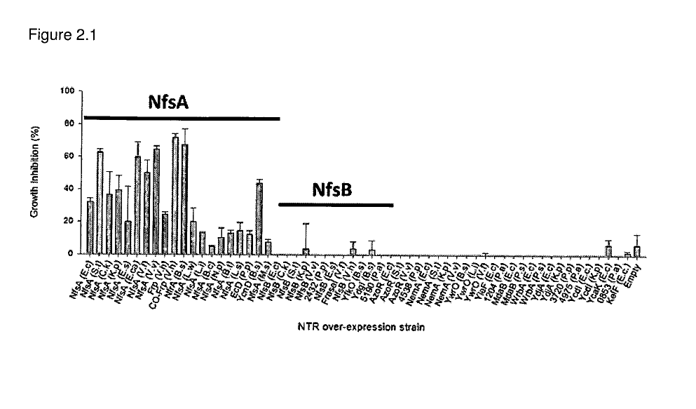

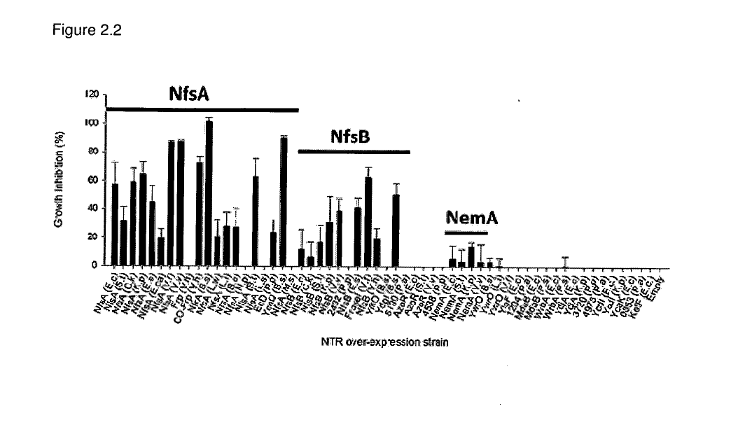

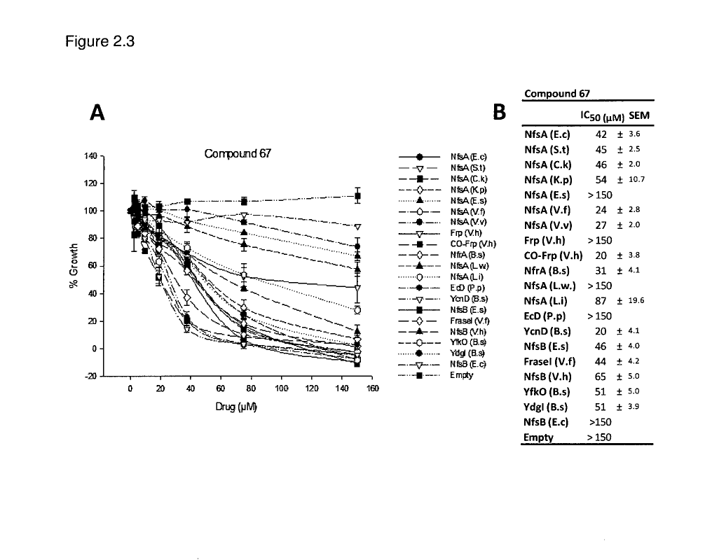

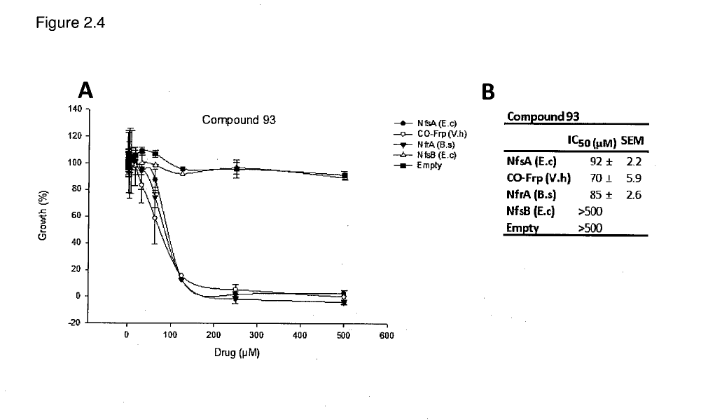

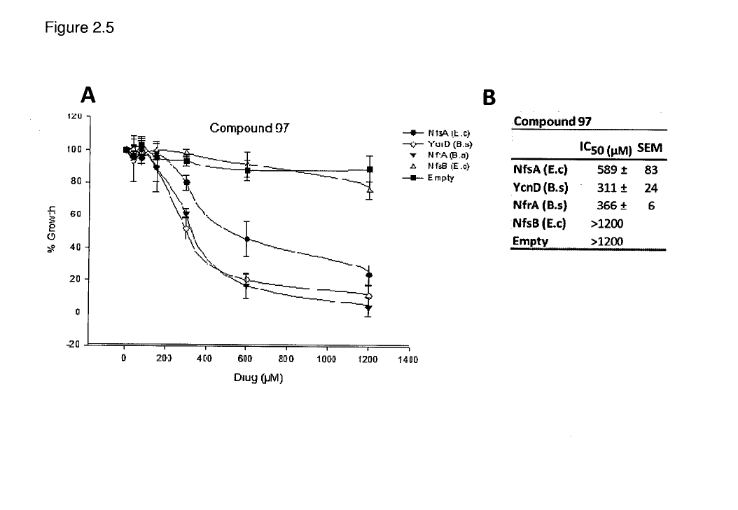

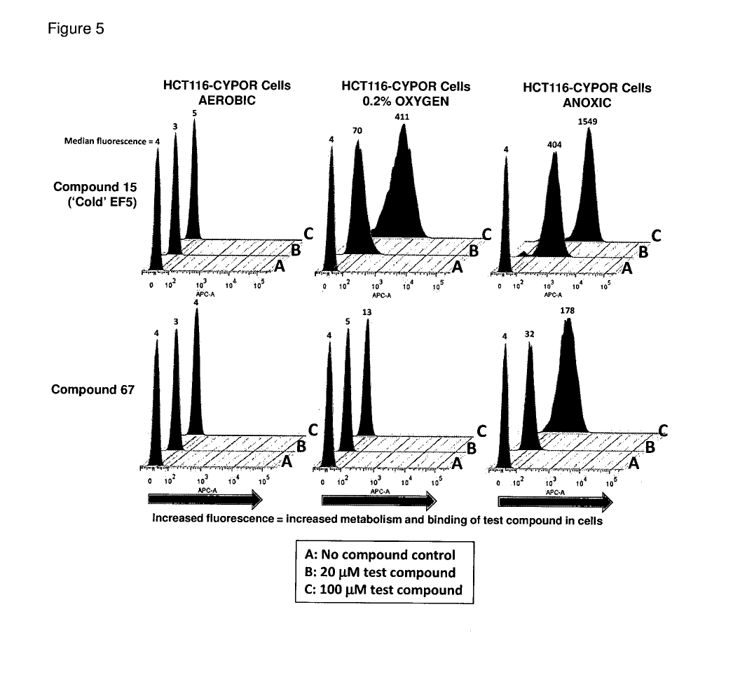

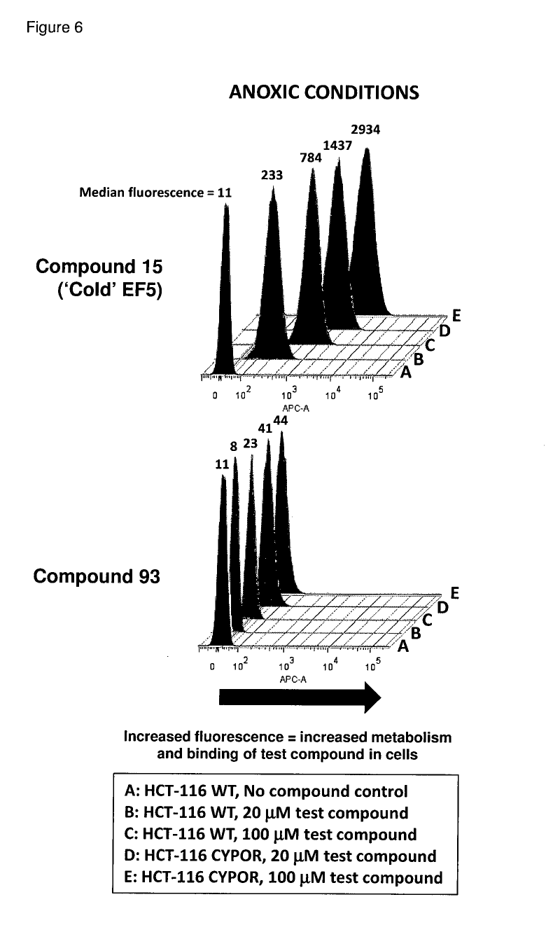

Compounds of use in the present invention are selectively metabolised by bacterial nitroreductases (such as nitroreductase enzymes is expressed by a wild type or mutant variant of E coli NfsA) and are substantially insensitive to metabolism in mammalian cells under either oxic or hypoxic conditions. Examples 2, 4 (FIGS. 2, 2.1, 2.2) 4.1, 4.2 (FIG. 2.3, 2.4, 2.5), 4.3, 5, 6 (FIG. 5), 7 (FIG. 6) and 8 (FIG. 7, 8, 9, 10, 11) demonstrate that compounds of use in the invention have one-electron reduction potentials sufficiently low to be substantially insensitive to metabolism and retention in human tumours experiencing pathological levels of hypoxia. This surprising finding would not have been expected when considering the background signal caused by known imaging agents such as EF5 and demonstrated in Example 8. Therefore the inventors have found and demonstrated that compounds of use in the invention are excellent substrates for bacterial nitroreductase metabolism under oxic conditions. This property provides retention of the reduction metabolites exclusively in bacterial nitroreductase expressing cells as imaged by FACS analysis. When ¹⁸F radiolabelled compounds of the invention are utilised, selective, rapid, reproducible and quantitative non-invasive imaging and/or monitoring of bacterial nitroreductase-expressing cell and/or biological agent distribution and amplitude in a patient or animal over time is made possible. This feature of the invention allows for optimised signal to noise when imaging nitroreductase expressing cells or biological agents. This has particular utility in imaging vectors for gene therapy applications.

In a particular embodiment, the radiolabelled compound is used to radioimage a subject using an imaging technique such as Positron Emission Tomography (PET), micro-Positron Emission Tomography (micro-PET) or Single Photon Emission Tomography (SPECT). The compound may contain a positron-emitting nuclide such as ¹⁵O, ¹³N, ¹¹C, ¹²⁴I, ⁷⁶Br and ¹⁸F (for PET) or a gamma-emitting nuclide such as ^{99m}Tc, ⁶⁷Ga, ¹¹¹In and ¹²³I (for SPECT). ¹⁸F is referred to throughout this specification as an exemplary radiolabel. However, it will be understood by one of skill in the art that other radiolabels including those mentioned above may have utility in place of ¹⁸F. Compounds which contain other radiolabels are intended to be included within the scope of the invention.

Further, in their “cold” or non-radiolabelled form at high dose, compounds of use in the invention are metabolised by the expressed NTRs and the cytotoxic metabolites selectively ablate the nitroreductase expressing cell or biological agent with minimal cytotoxicity to neighbouring cells. In a particular embodiment, this feature allows for the selective eradication of a replicating biological vector. This is achieved by using reduction metabolites with a substantially minimal or zero bystander effect.

A direct correlation between the intensity of fluorescence observed for immunohistochemical detection of EF5 binding in tumour xenografts using “high dose” cold EF5 and the intensity of 18-F PETsignal observed during small animal PET imaging of tumour xenografts when using “low dose” 18-F labeled EF5 has been reported [Koch et al., Eur J Nucl Med Mol Imaging, 2010, 37: 2048-2059; Yapp et al., Br J Urol Int, 2007, 99: 1154-1160], such that it is known to one skilled in the art that immunohistochemical evidence of probe binding in vivo is sufficient to predict an 18-F PET signature of probe binding.

### Dual Use of the Compound for Imaging and Single Cell Ablation

The inventors have surprisingly found that the class of compounds defined herein as part of the invention can be used in their radiolabelled and “cold” forms for imaging and single cell ablation respectively. This dual utility has major benefits in both a clinical and research context. Since the radiolabelled compound and the cold compound are essentially the same compound (they differ only in the isotopic form of one of the nuclides), the imaging of the radiolabelled compound directly reports about the pharmacokinetics, tissue distribution and clearance of the cold version used for single cell ablation. In known systems, the imaging agent (for example EF5) and the prodrug (for example metronidazole) have to be tested separately against the nitroreductase expressing vector or biological agent to determine their metabolic characteristics and to determine the enzyme activity. In contrast, using a compound of the present invention will only require a single test as the radiolabelled compound would have substantially the same metabolic characteristics as the cold compound.

The compound structures referred to within this specification predominantly refer to the use of F as the cold nuclide in place of the radionuclide in the corresponding radiolabeled compound. It will be understood by one of skill in the art that other nuclides may have utility in place of F. For example ¹⁶O, ¹⁴N, ¹²C, ¹²⁶I, ⁷⁹Br, ¹⁹F, ⁹⁷Tc, ⁶⁹Ga, ¹¹⁴In and ¹²⁶I are of particular utility in for use in the cold compounds. Compounds which contain other nuclides to those exemplified in the specification are intended to be included within the scope of the invention.

The radiolabelled imaging agent and the corresponding non-radiolabelled cold compound may also be used to facilitate the directed evolution of bacterial nitroreductase for use in bacterial nitroreductase expressing vectors and/or biological agents. Using a compound that differs only in the labeled isotope has substantial benefits in reducing the time and effort that would otherwise be needed to evolve the bacterial nitroreductase to be effective against two separate compounds.

Compounds of the Invention have Decreased Response to Hypoxic Regions

The Y side chains labelled IIa to IIg, IIIa to IIIh and IVa to IVg have been previously validated in the context of 2-nitroimidazole-based hypoxia PET imaging agents as having suitable labelling chemistries including desirable properties for ease of probe preparation, imaging of the probe and tissue pharmacokinetics and clearance [Minn, H. et al Current Pharmaceutical Design, 2008, 14, 2932-2942]. The favourable properties associated with these side chains have been validated in other studies and would be expected by one skilled in the art to be imparted to the compounds of the present invention. The inventors have surprisingly found that the compounds of use in the present invention have unexpected desirable properties such as the decreased metabolism of the compound by human nitroreductase enzymes in hypoxic tumour regions and the relatively greater selectivity for metabolism by bacterial NTR enzymes. In particular the compounds of use in the present invention have a nitroheterocyclic or nitroaromatic substituent with a sufficiently low one-electron reduction potential to prevent metabolism by human enzymes in the hypoxic areas of a tumour. Neither the known 2-nitroimidazoles or the compounds of the present invention are metabolised by human enzymes under oxic conditions unless a bacterial NTR is expressed.

Accordingly, in one aspect, the invention provides a method of selecting a nitroheterocyclic or nitroaromatic compound for use in a method of imaging and/or ablation of a bacterial nitroreductase-expressing cell and/or a bacterial nitroreductase-expressing biological agent, the method comprising:
a. under both oxic and hypoxic conditions, separately measuring the sensitivity of the compound to metabolism by a human nitroreductase enzyme and a bacterial nitroreductase enzyme; and
b. selecting the compound if it is
   i. substantially insensitive to metabolism by a human nitroreductase enzyme; and
   ii. metabolised by a bacterial nitroreductase enzyme.

In a particular embodiment of the invention, there is provided a compound of general formula I or V where the Y side chain is IIg, IIIg or IVg. Compounds with this side chain have particular utility as imaging agents because such compounds are believed to be quickly and easily removed from the body during and after administration therefore minimizing background radiosignal readily allowing for detection of the bound form.

Without wishing to be bound by theory, it is believed that this side chain has an optimal level of hydrophilicity and imparts renal clearance to the compound, such that much of the unbound radiolabelled compound is cleared by the kidneys into the bladder. In a clinical setting, this property enables the patient to ‘void’ the bladder by drinking a reasonable quantity of water which results in the compound being cleared by the body. The remaining radiolabelled compound that has been metabolized by a bacterial nitroreductase and therefore irreversibly bound in tissue can then be imaged free of a background of unbound radiolabelled compound. This enables bacterial nitroreductase positive areas in the central body cavity to be effectively imaged.

### Single Cell Ablation

In a particular embodiment, the invention comprises “cold” or non-radioactive compounds which contain a non-radioactive isotope. These compounds have particular utility for selective ablation of nitroreductase expressing cells and/or biological agents. The ability to ablate individual cells expressing a cognate NTR without localised damage to neighbouring tissue is seen as a valuable safety control for enabling the elimination of the NTR-expressing vector in the matrix, cells or tissues should this be deemed necessary. The ability to control viral (VDEPT) or bacterial (BDEPT) infection is an additional biosafety feature and is considered to be a desirable design feature in replicating biological vectors.

Once metabolised by an NTR enzyme, the compounds of use in the invention may suppress or ablate a target cell and/or biological agent. The target cell/biological agent that is ablated may either directly express a nitroreductase or be present in the local microenvironment of the cell/biological agent that expresses an NTR. It is envisaged that the target cell/biological agent local tissue microenvironment may be colonised regionally by tumour-tropic bacterium (e.g. Clostridium sp, Salmonella sp, Bifidobacterium sp).

In a particular embodiment, the cell or biological agent is a stem cell or a vector that expresses an NTR. This use enables the control and selective ablation of introduced cells to prevent uncontrolled growth (e.g. tumour formation) or to restrict the growth of therapeutic cells to a particular location. This use, especially combined with the use of the NTR-metabolised imaging probe represents a useful technology to improve the accuracy and ensure the safety of novel treatments, often with unknown outcomes.

The ability of activated compounds to diffuse from the site of production and ablate unmodified cells in the local microenvironment is termed the “bystander effect” and is an important determinant of the overall efficacy of any prodrug activating system. Bystander effect efficiency (BEE) can be quantified according to methods described in Wilson et al, 2002, Cancer Res. 62:1425-1432. A BEE value of less than about 15%, less than about 10%, less than about 5%, less than about 1% or zero is considered “substantially minimal”. A BEE value of greater than about 50%, about 60%, about 70% is considered “substantial”.

Prodrug conditional single cell ablation may be employed to improve the sensitivity of cells (such as transplanted stem cells, engrafted hematopoietic stem cells or genetically modified immune cells) to cell ablation by use of a vector selective for the cell or by direct modification of the cell to express an NTR of the invention. This minimises the unpredictable side effects that may result from uncontrolled spread of the modified cells. Methods that may benefit from the use of NTR expressing vectors/cells include ex-vivo transfection with systemic reintroduction, or cell selective in vivo methods of gene transfer. Such techniques have use in the treatment of a wide variety of human diseases, including Parkinson's disease, Alzheimer's disease, stroke, heart disease, rheumatological diseases and diseases treated by stem-cell transplantation.

A dose of the “cold” compound is used to perform the ablation where the dose is substantially higher than the dose of the radioactive compound used for imaging. The compound will be present in the tumour at sufficiently high concentrations that the NTR metabolism results in cytotoxicity of the NTR-expressing cell or biological agent. The reduction metabolites have a substantially minimal or zero bystander effect so that the adjacent cells are not ablated or harmed. The inventors have shown in a previous application (PCT/NZ2011/000137 incorporated herein by reference) that 2-NI probes (e.g. EF5), when administered at a high dose when compared to the dose used for the purpose of PET imaging, can selectively ablate NTR-expressing cells. The dosage required to enable ablation is preferably approximately the maximum tolerated dose (MTD) for the subject. “High dose” may also relate to the achievable concentrations in human plasma using ‘cold’ (radiolabel-free) EF5 administration. At 0.7 mM-hr cold EF5 provides 90% loss of viability for nfsA expressing HCT116 cells. A concentration-time of 0.89 mM-hr is readily achieved in human plasma following administration of cold EF5 (9 mg/kg). A dose of 21 mg/kg can be safely injected without any toxicities and will provide a plasma AUC of 2 mM-hr (Koch et al., Can Chemother Pharmacol, 2001, 48:177-187). A 1000-fold lower concentration (0.1%) of radiolabelled drug ¹⁸F-EF5 is administered for PET imaging and will not result in cell ablation (Koch et al., 2010, Eur J Nucl Med Mol Imaging, 37:2048-2059). In a particular embodiment, the “high” dose of the compound administered for the purposes of ablation is approximately 10 times, 100 times, 1000 times or 10000 times or greater than the dose of the compound typically used for the purposes of imaging. A “high” dose will be typically in the range of 1 to 30 mg/kg of body weight.

### Preferred Compounds of the Present Invention

The invention provides compounds of formula I and V as defined above.

¹⁸F PET Agents:
[¹⁸F]-2-(5-nitro-1H-imidazol-1-yl)-N-(2,2,2-trifluoroethyl)acetamide (47)
[¹⁸F]-2-(5-nitro-1H-imidazol-1-yl)-N-(2,2,3,3,3-pentafluoropropyl)acetamide (48)
[¹⁸F]-3-fluoro-2-(4-((5-nitro-1H-imidazol-1-yl)methyl)-1H-1,2,3-triazol-1-yl)propan-1-ol (52)
[¹⁸F]-2-(2-methyl-5-nitro-1H-imidazol-1-yl)-N-(2,2,2-trifluoroethyl)acetamide (73)
[¹⁸F]-2-(2-methyl-5-nitro-1H-imidazol-1-yl)-N-(2,2,3,3,3-pentafluoropropyl)acetamide (74)
[¹⁸F]-3-fluoro-2-(4-((2-methyl-5-nitro-1H-imidazol-1-yl)methyl)-1H-1,2,3-triazol-1-yl)propan-1-ol (78)
[¹⁸F]-2-(4-nitro-1H-imidazol-1-yl)-N-(2,2,2-trifluoroethyl)acetamide (99)
[¹⁸F]-2-(4-nitro-1H-imidazol-1-yl)-N-(2,2,3,3,3-pentafluoropropyl)acetamide (100)
[¹⁸F]-3-fluoro-2-(4-((4-nitro-1H-imidazol-1-yl)methyl)-1H-1,2,3-triazol-1-yl)propan-1-ol (104)
[¹⁸F]-2-(2-nitro-1H-pyrrol-1-yl)-N-(2,2,2-trifluoroethyl)acetamide (125)
[¹⁸F]-2-(2-nitro-1H-pyrrol-1-yl)-N-(2,2,3,3,3-pentafluoropropyl)acetamide (126)
[¹⁸F]-3-fluoro-2-(4-((2-nitro-1H-pyrrol-1-yl)methyl)-1H-1,2,3-triazol-1-yl)propan-1-ol (130)
[¹⁸F]-3-fluoro-2-(4-(4-nitrobenzyl)-1H-1,2,3-triazol-1-yl)propan-1-ol (156)
[¹⁸F]-2-(2-(fluoromethyl)-4-nitro-1H-imidazol-1-yl)-N-(2,2,3,3,3-pentafluoropropyl)acetamide (164)
[¹⁸F]-2-(5-(fluoromethyl)-4-nitro-1H-imidazol-1-yl)-N-(2,2,3,3,3-pentafluoropropyl)acetamide (172)
[¹⁸F]-2-(2-(fluoromethyl)-5-nitro-1H-imidazol-1-yl)-N-(2,2,3,3,3-pentafluoropropyl)acetamide (180)
[¹⁸F]-2-(2-(fluoromethyl)-5-nitro-1H-pyrrol-1-yl)-N-(2,2,3,3,3-pentafluoropropyl)acetamide (196)
[¹⁸F]-2-(2-(fluoromethyl)-4-nitrophenyl)-N-(2,2,3,3,3-pentafluoropropyl)acetamide (212)

### Cold PET Agents:

2-(5-nitro-1H-imidazol-1-yl)-N-(2,2,2-trifluoroethyl)acetamide (40)
2-(5-nitro-1H-imidazol-1-yl)-N-(2,2,3,3,3-pentafluoropropyl)acetamide (41)
3-fluoro-2-(4-((5-nitro-1H-imidazol-1-yl)methyl)-1H-1,2,3-triazol-1-yl)propan-1-ol (45)
2-(2-methyl-5-nitro-1H-imidazol-1-yl)-N-(2,2,2-trifluoroethyl)acetamide (66)
2-(2-methyl-5-nitro-1H-imidazol-1-yl)-N-(2,2,3,3,3-pentafluoropropyl)acetamide (67)
3-fluoro-2-(4-((2-methyl-5-nitro-1H-imidazol-1-yl)methyl)-1H-1,2,3-triazol-1-yl)propan-1-ol (71)
2-(4-nitro-1H-imidazol-1-yl)-N-(2,2,2-trifluoroethyl)acetamide (92)
2-(4-nitro-1H-imidazol-1-yl)-N-(2,2,3,3,3-pentafluoropropyl)acetamide (93)
3-fluoro-2-(4-((4-nitro-1H-imidazol-1-yl)methyl)-1H-1,2,3-triazol-1-yl)propan-1-ol (97)
2-(2-nitro-1H-pyrrol-1-yl)-N-(2,2,2-trifluoroethyl)acetamide (118)
2-(2-nitro-1H-pyrrol-1-yl)-N-(2,2,3,3,3-pentafluoropropyl)acetamide (119)
3-fluoro-2-(4-((2-nitro-1H-pyrrol-1-yl)methyl)-1H-1,2,3-triazol-1-yl)propan-1-ol (123)
3-fluoro-2-(4-(4-nitrobenzyl)-1H-1,2,3-triazol-1-yl)propan-1-ol (149)

### Precursors:

3-hydroxy-2-(4-((5-nitro-1H-imidazol-1-yl)methyl)-1H-1,2,3-triazol-1-yl)propyl methanesulfonate (31)
N-(2,2-difluorovinyl)-2-(5-nitro-1H-imidazol-1-yl)acetamide (33)
2-(5-nitro-1H-imidazol-1-yl)-N-(2,3,3-trifluoroallyl)acetamide (34)
3-hydroxy-2-(4-((5-nitro-1H-imidazol-1-yl)methyl)-1H-1,2,3-triazol-1-yl)propyl 4-methylbenzenesulfonate (38)
3-hydroxy-2-(4-((2-methyl-5-nitro-1H-imidazol-1-yl)methyl)-1H-1,2,3-triazol-1-yl)propyl methanesulfonate (57)
N-(2,2-difluorovinyl)-2-(2-methyl-5-nitro-1H-imidazol-1-yl)acetamide (59)
2-(2-methyl-5-nitro-1H-imidazol-1-yl)-N-(2,3,3-trifluoroallyl)acetamide (60)
3-hydroxy-2-(4-((2-methyl-5-nitro-1H-imidazol-1-yl)methyl)-1H-1,2,3-triazol-1-yl)propyl 4-methylbenzenesulfonate (64)
3-hydroxy-2-(4-((4-nitro-1H-imidazol-1-yl)methyl)-1H-1,2,3-triazol-1-yl)propyl methanesulfonate (83)
N-(2,2-difluorovinyl)-2-(4-nitro-1H-imidazol-1-yl)acetamide (85)
2-(4-nitro-1H-imidazol-1-yl)-N-(2,3,3-trifluoroallyl)acetamide (86)
3-hydroxy-2-(4-((4-nitro-1H-imidazol-1-yl)methyl)-1H-1,2,3-triazol-1-yl)propyl 4-methylbenzenesulfonate (90)
3-hydroxy-2-(4-((2-nitro-1H-pyrrol-1-yl)methyl)-1H-1,2,3-triazol-1-yl)propyl methanesulfonate (109)
N-(2,2-difluorovinyl)-2-(2-nitro-1H-pyrrol-1-yl)acetamide (111)
2-(2-nitro-1H-pyrrol-1-yl)-N-(2,3,3-trifluoroallyl)acetamide (112)
3-hydroxy-2-(4-((2-nitro-1H-pyrrol-1-yl)methyl)-1H-1,2,3-triazol-1-yl)propyl 4-methylbenzenesulfonate (116)
3-hydroxy-2-(4-(4-nitrobenzyl)-1H-1,2,3-triazol-1-yl)propyl methanesulfonate (135)
3-hydroxy-2-(4-(4-nitrobenzyl)-1H-1,2,3-triazol-1-yl)propyl 4-methylbenzenesulfonate (142)
3-hydroxy-2-(4-((5-nitro-1H-imidazol-1-yl)methyl)-1H-1,2,3-triazol-1-yl)propyl 2-nitrobenzenesulfonate (321)
3-((methylsulfonyl)oxy)-2-(4-((5-nitro-1H-imidazol-1-yl)methyl)-1H-1,2,3-triazol-1-yl)propyl acetate (325)
2-(4-((5-nitro-1H-imidazol-1-yl)methyl)-1H-1,2,3-triazol-1-yl)-3-(tosyloxy)propyl acetate (329)
2-(4-((5-nitro-1H-imidazol-1-yl)methyl)-1H-1,2,3-triazol-1-yl)-3-(((2-nitrophenyl)sulfonyl)oxy)propyl acetate (333)
3-hydroxy-2-(4-((2-methyl-5-nitro-1H-imidazol-1-yl)methyl)-1H-1,2,3-triazol-1-yl)propyl 2-nitrobenzenesulfonate (338)
2-(4-((2-methyl-5-nitro-1H-imidazol-1-yl)methyl)-1H-1,2,3-triazol-1-yl)-3-((methylsulfonyl)oxy)propyl acetate (342)
2-(4-((2-methyl-5-nitro-1H-imidazol-1-yl)methyl)-1H-1,2,3-triazol-1-yl)-3-(tosyloxy)propyl acetate (346)
2-(4-((2-methyl-5-nitro-1H-imidazol-1-yl)methyl)-1H-1,2,3-triazol-1-yl)-3-(((2-nitrophenyl)sulfonyl)oxy)propyl acetate (350)
3-hydroxy-2-(4-((4-nitro-1H-imidazol-1-yl)methyl)-1H-1,2,3-triazol-1-yl)propyl 2-nitrobenzenesulfonate (355)
3-((methylsulfonyl)oxy)-2-(4-((4-nitro-1H-imidazol-1-yl)methyl)-1H-1,2,3-triazol-1-yl)propyl acetate (359)
2-(4-((4-nitro-1H-imidazol-1-yl)methyl)-1H-1,2,3-triazol-1-yl)-3-(tosyloxy)propyl acetate (363)
2-(4-((4-nitro-1H-imidazol-1-yl)methyl)-1H-1,2,3-triazol-1-yl)-3-(((2-nitrophenyl)sulfonyl)oxy)propyl acetate (367)
3-hydroxy-2-(4-((2-nitro-1H-pyrrol-1-yl)methyl)-1H-1,2,3-triazol-1-yl)propyl 2-nitrobenzenesulfonate (372)
3-((methylsulfonyl)oxy)-2-(4-((2-nitro-1H-pyrrol-1-yl)methyl)-1H-1,2,3-triazol-1-yl)propyl acetate (376)
2-(4-((2-nitro-1H-pyrrol-1-yl)methyl)-1H-1,2,3-triazol-1-yl)-3-(tosyloxy)propyl acetate (380)
2-(4-((2-nitro-1H-pyrrol-1-yl)methyl)-1H-1,2,3-triazol-1-yl)-3-(((2-nitrophenyl)sulfonyl)oxy)propyl acetate (384)
3-hydroxy-2-(4-(4-nitrobenzyl)-1H-1,2,3-triazol-1-yl)propyl 2-nitrobenzenesulfonate (389)
3-((methylsulfonyl)oxy)-2-(4-(4-nitrobenzyl)-1H-1,2,3-triazol-1-yl)propyl acetate (393)
2-(4-(4-nitrobenzyl)-1H-1,2,3-triazol-1-yl)-3-(tosyloxy)propyl acetate (397)
2-(4-(4-nitrobenzyl)-1H-1,2,3-triazol-1-yl)-3-(((2-nitrophenyl)sulfonyl)oxy)propyl acetate (401)

In a particular embodiment of the invention, there is provided a method of imaging and/or ablation comprising the use of:
a. a radiolabelled compound according to formula 104:

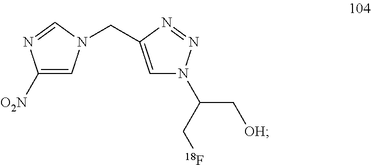 [SMILES: O=[N+]([O-])c1cn(Cc2cn(C(CO)C[18F])nn2)cn1]

and/or
b. a compound according to formula 97:

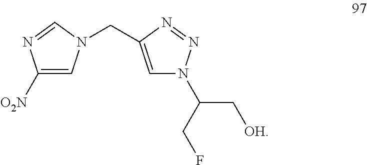 [SMILES: O=[N+]([O-])c1cn(Cc2cn(C(CO)CF)nn2)cn1]

These compounds may be prepared by the novel precursor compound according to formula 367:

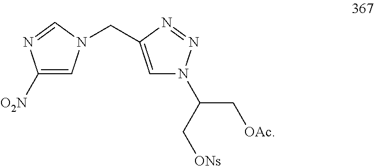

### Structures of Preferred Compounds of the Present Invention

The invention provides novel compounds that have particular utility as imaging agents and/or as compounds to carry out single cell ablation. The invention also provides precursor compounds to make these imaging/single cell ablation compounds. Preferred compounds of use in the invention are outlined below.

Mesylate, tosylate, nosylate and alkene radiolabelling precursors, bearing either unprotected or acetate-protected alcohol substituents, for the preparation of ¹⁸F-labelled 5-nitroimidazole PET imaging agents for the detection of bacterial nitroreductase expression

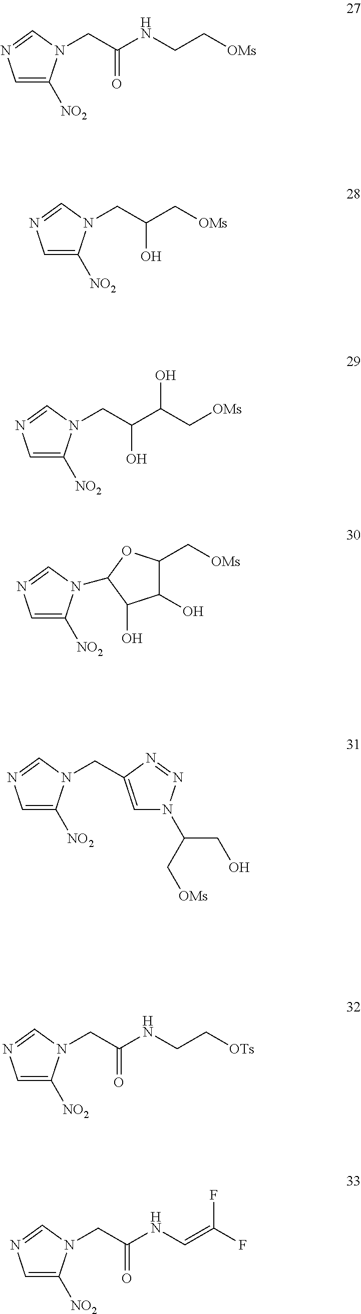

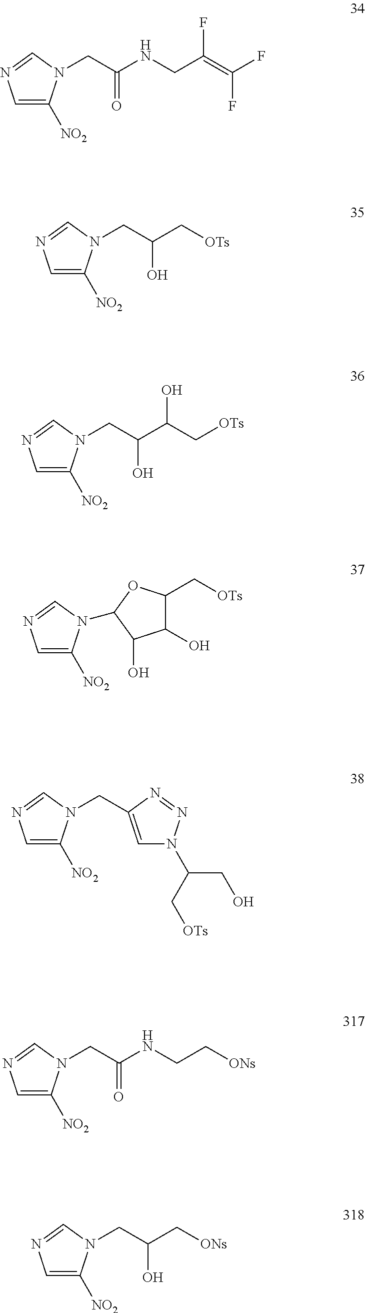

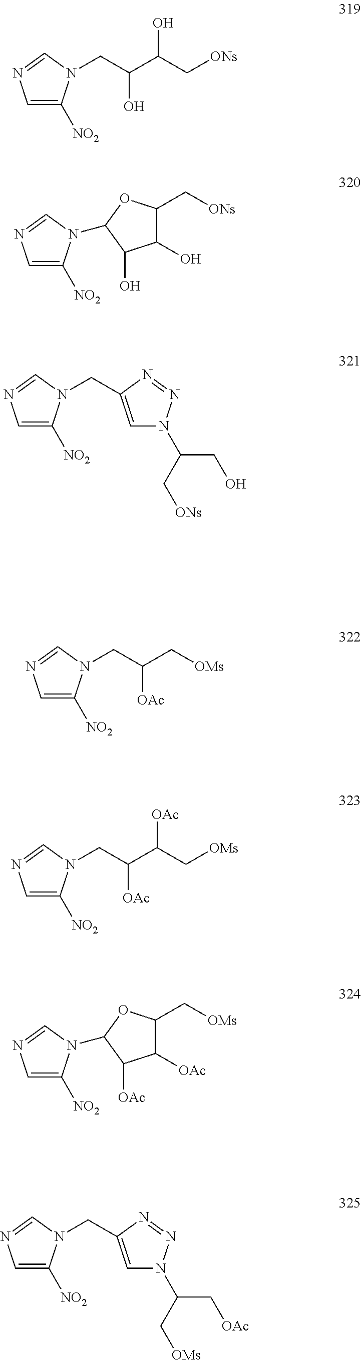

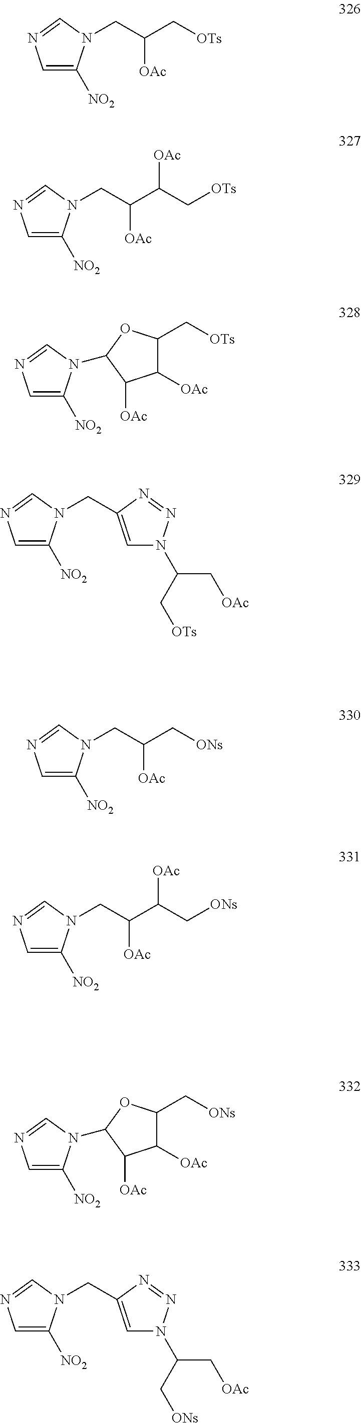

Non-radiolabelled (cold) examples of 5-nitroimidazole PET imaging agents for use at high dose to perform single cell ablation of bacterial nitroreductase expressing cells or biological agents (including viruses and bacteria) and for immunohistochemical detection of bacterial nitroreductase expression through antibody detection of trifluoro and pentafluoro side chain epitopes following tissue biopsy (for compounds 40 and 41 respectively)

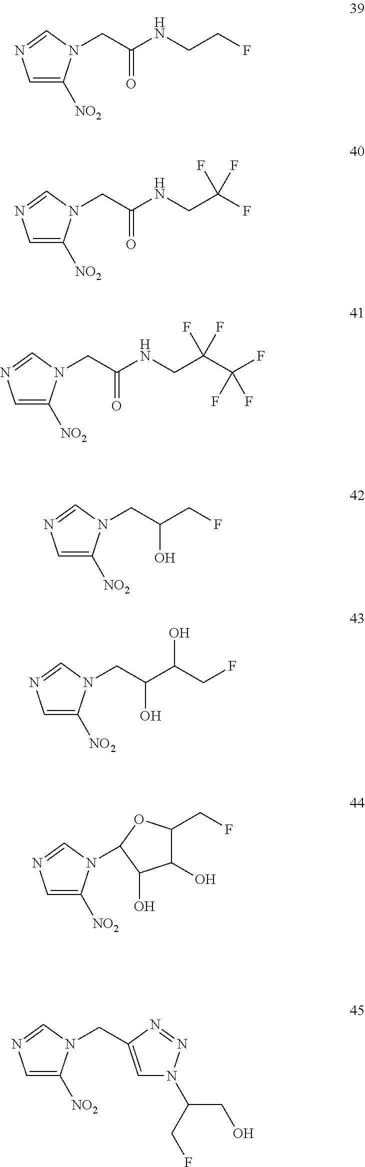

¹⁸F-labelled 5-nitroimidazole PET imaging agents for the detection of bacterial nitroreductase expression:

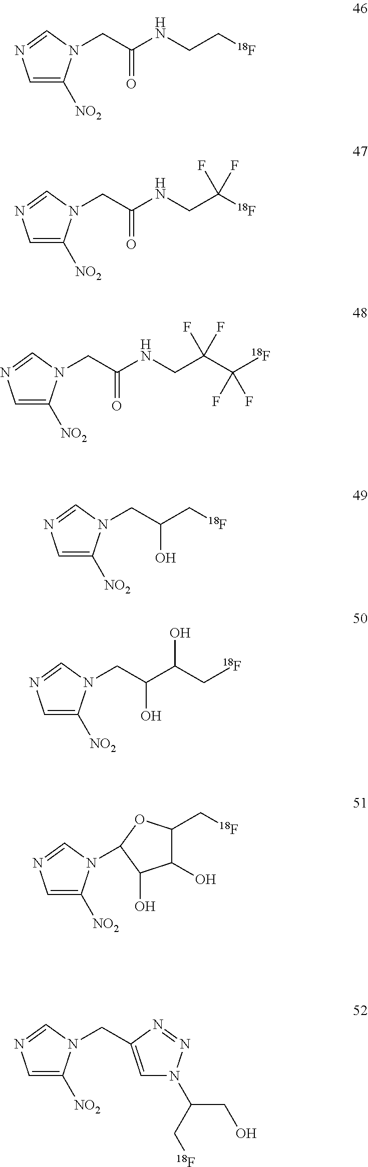

Mesylate, tosylate, nosylate and alkene radiolabelling precursors, bearing either unprotected or acetate-protected alcohol substituents, for the preparation of ¹⁸F-labelled 2-methyl-5-nitroimidazole PET imaging agents for the detection of bacterial nitroreductase expression:

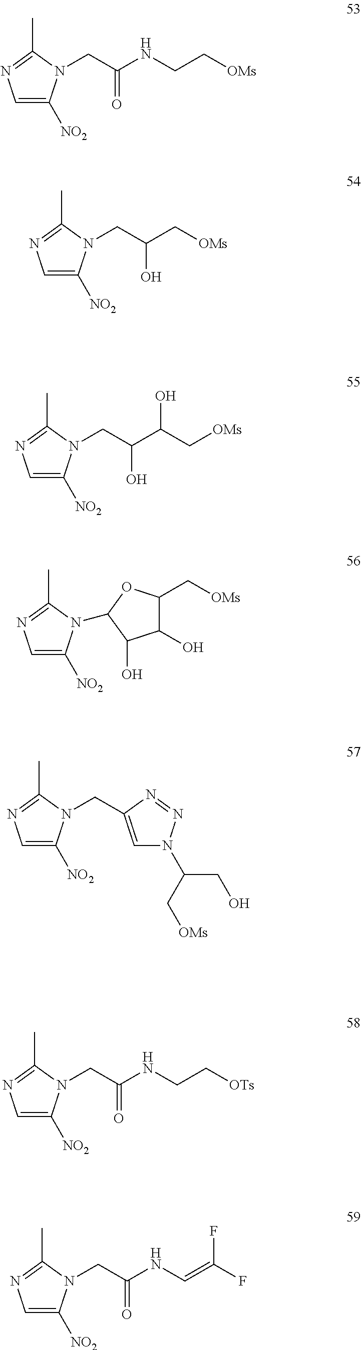

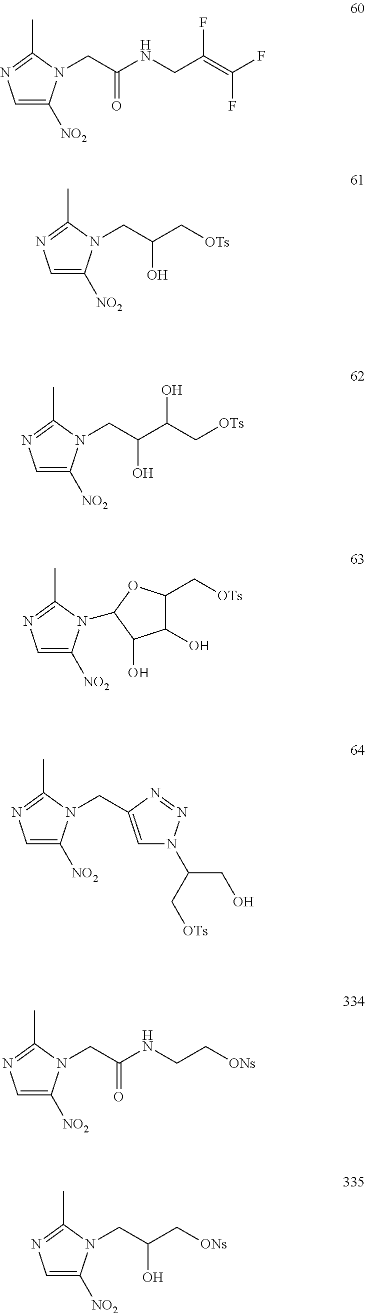

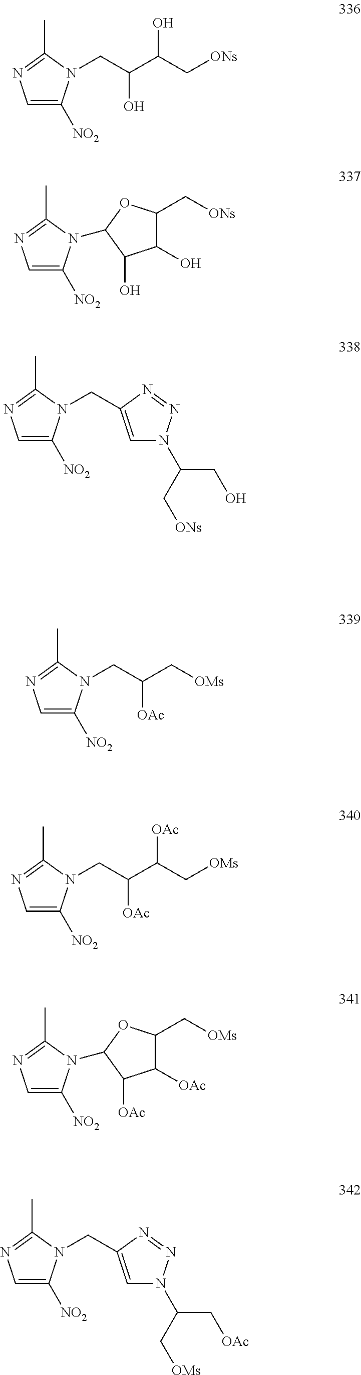

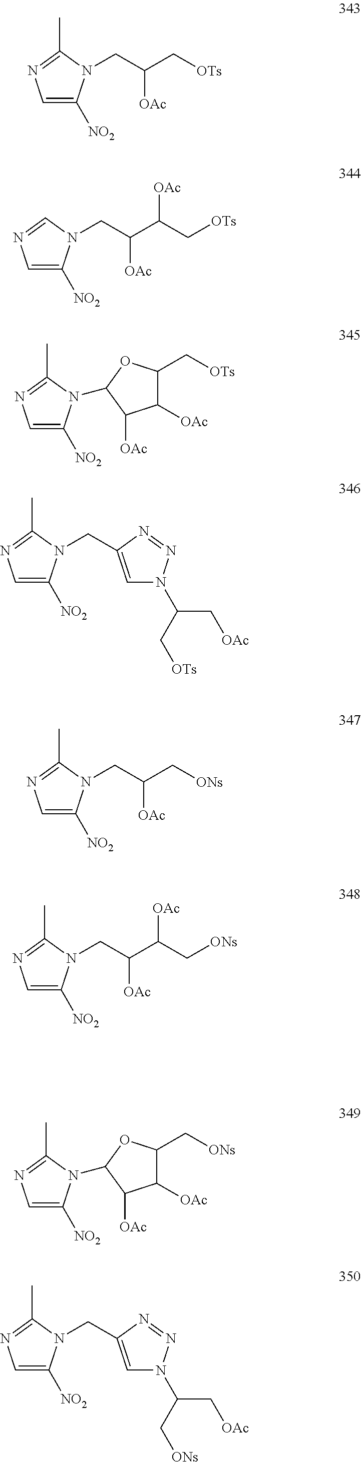

Non-radiolabelled (cold) examples of 2-methyl-5-nitroimidazole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy (for compounds 66 and 67 respectively):

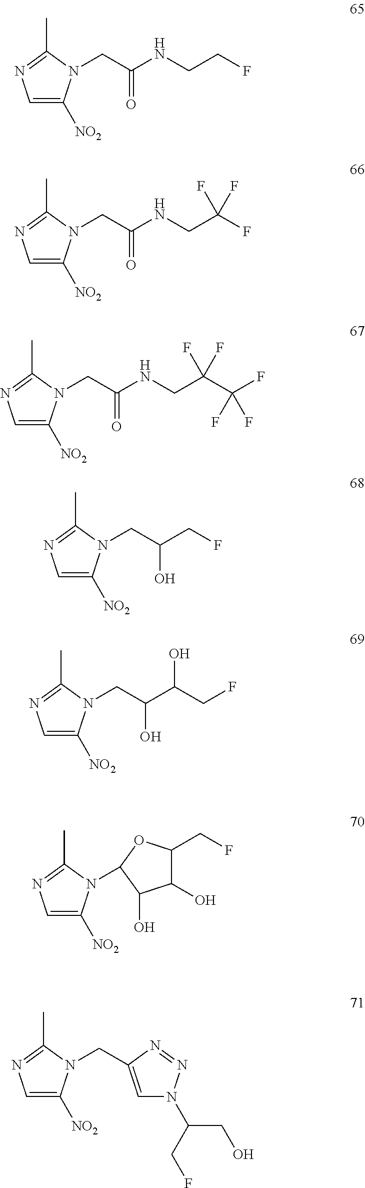

It should be noted that the structure of compound 68 has been previously disclosed as being a potential antibiotic [Cen, Junda; Zhong, Huijuan. PCT Int. Appl. 2006, WO 2006058457 A1]. However, the inventors have unexpectedly recognised its potential for use as a compound in imaging and single cell ablation methods.

¹⁸F-labelled 2-methyl-5-nitroimidazole PET imaging agents for the detection of bacterial nitroreductase expression:

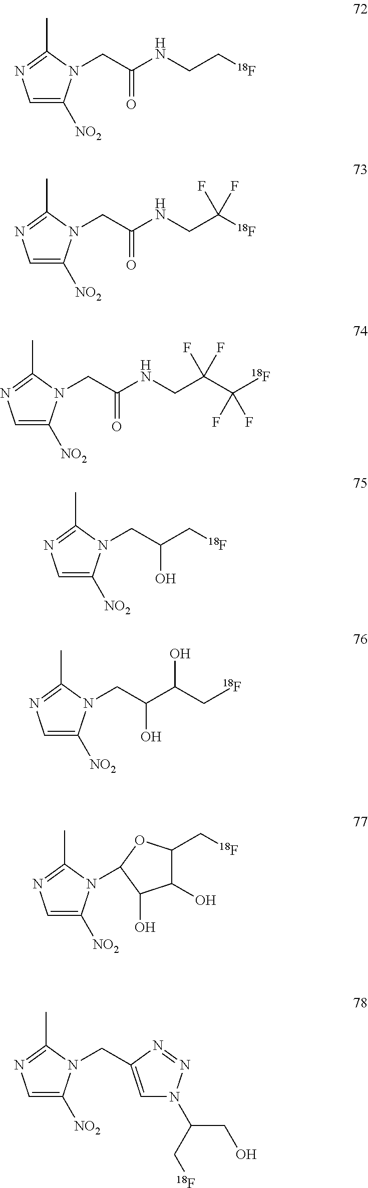

Mesylate, tosylate, nosylate and alkene radiolabelling precursors, bearing either unprotected or acetate-protected alcohol substituents, for the preparation of ¹⁸F-labelled 4-nitroimidazole PET imaging agents for the detection of bacterial nitroreductase expression:

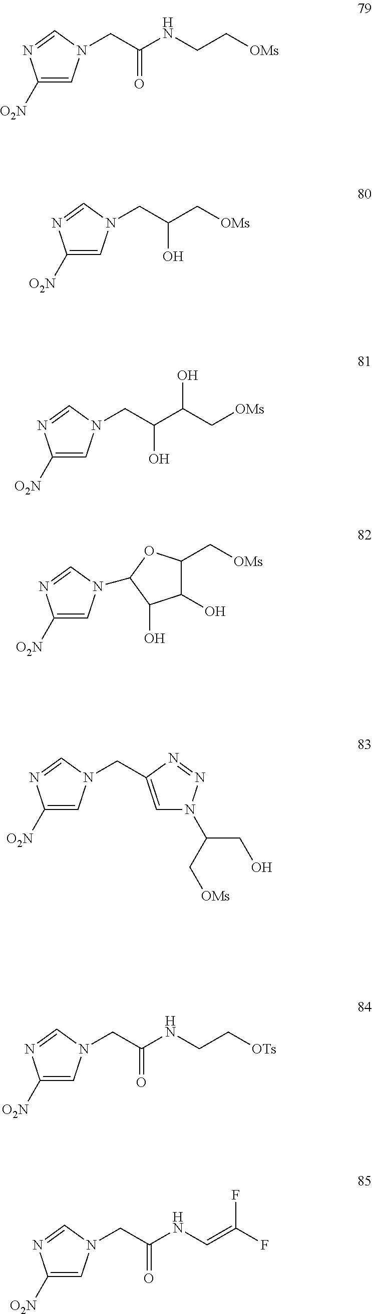

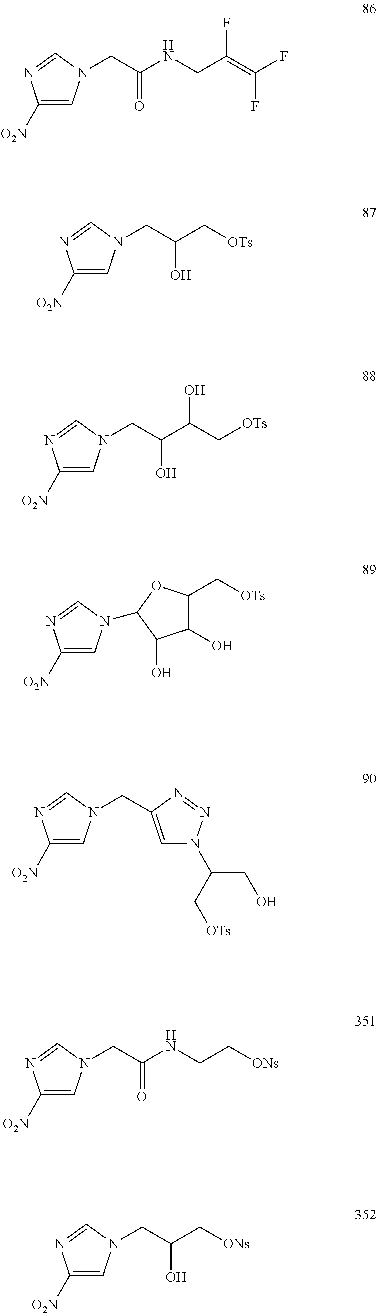

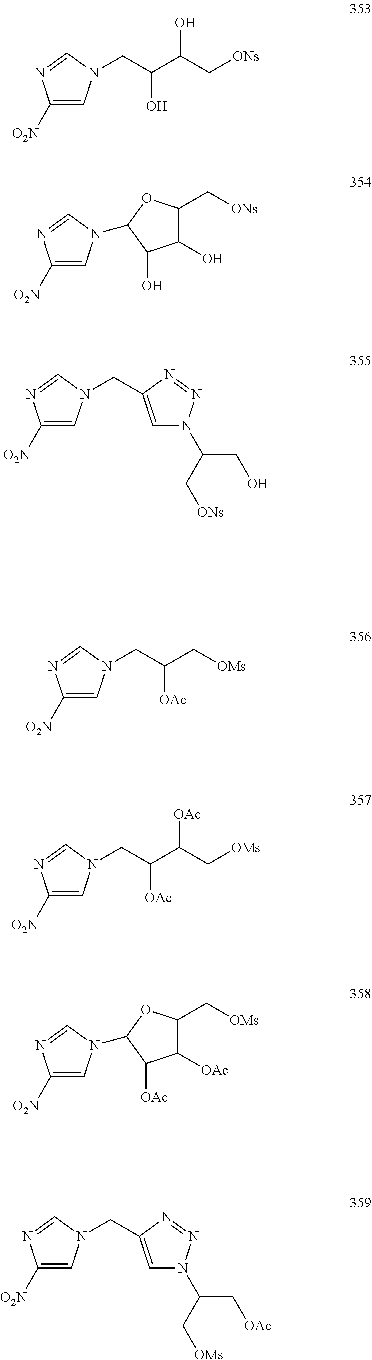

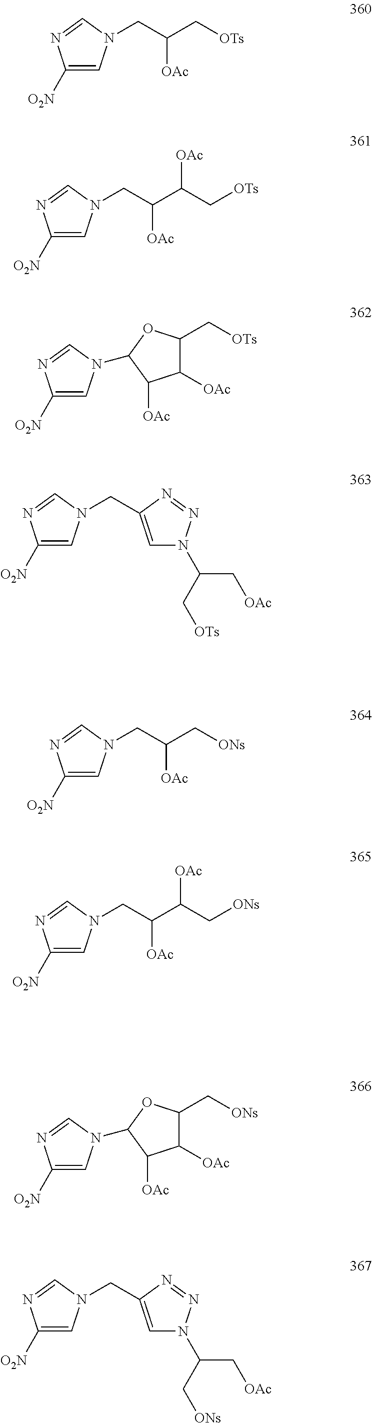

Non-radiolabelled (cold) examples of 4-nitroimidazole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy (for compounds 92 and 93 respectively):

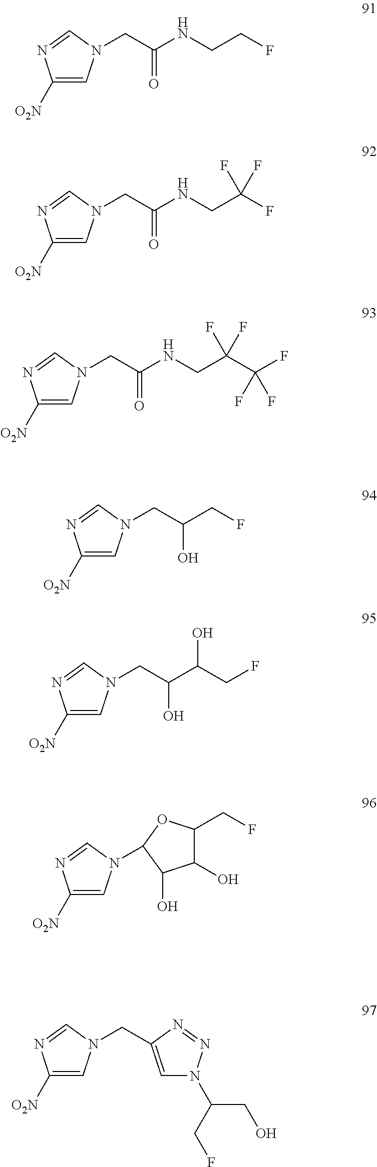

¹⁸F-labelled 4-nitroimidazole PET imaging agents for the detection of bacterial nitroreductase expression:

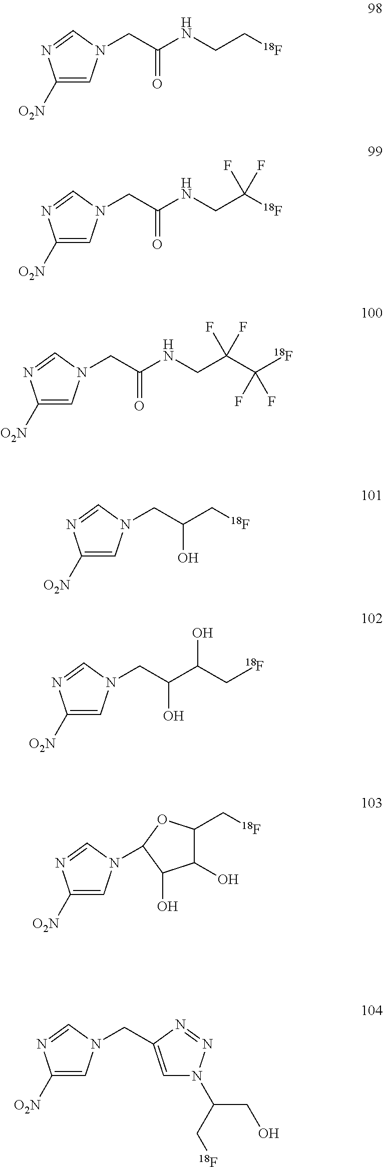

Mesylate, tosylate, nosylate and alkene radiolabelling precursors, bearing either unprotected or acetate-protected alcohol substituents, for the preparation of ¹⁸F-labelled 2-nitropyrrole PET imaging agents for the detection of bacterial nitroreductase expression:

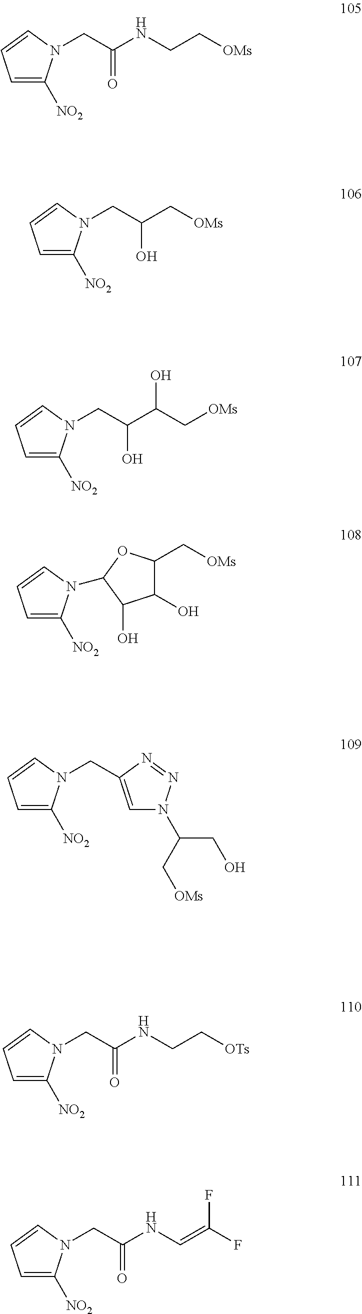

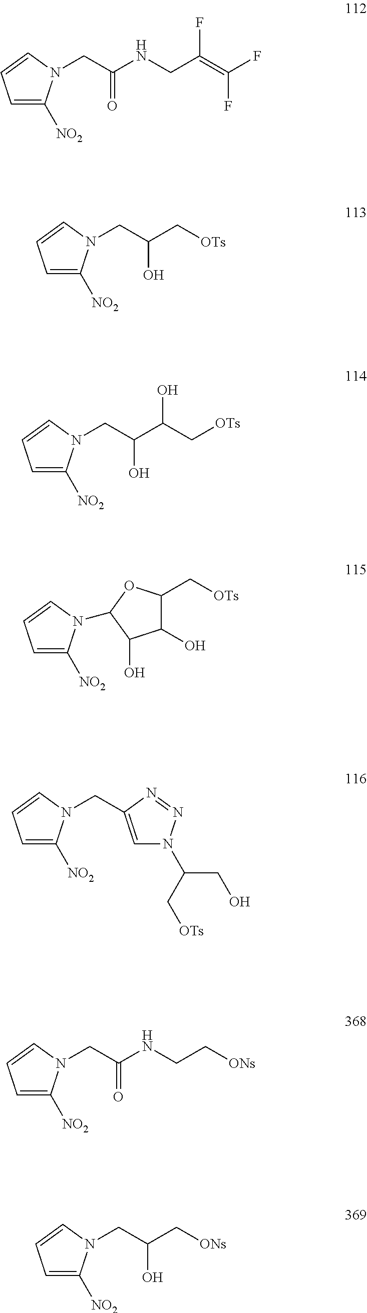

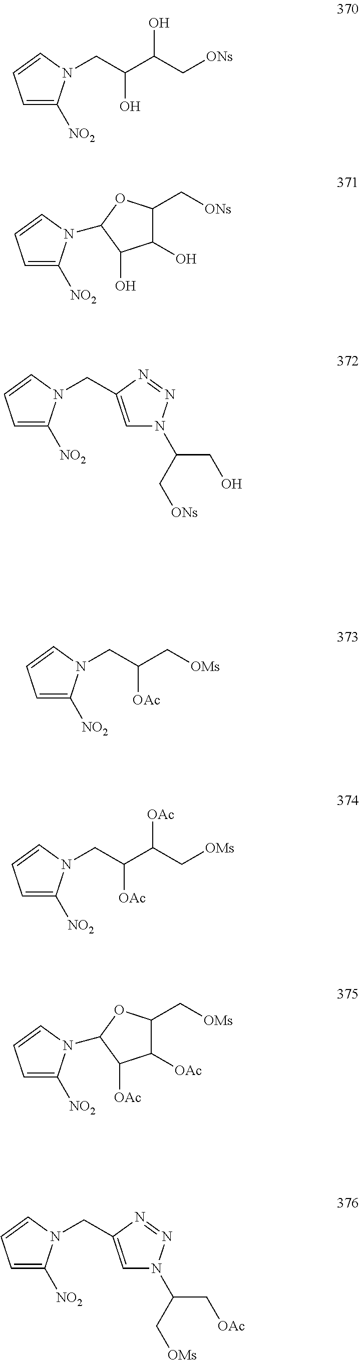

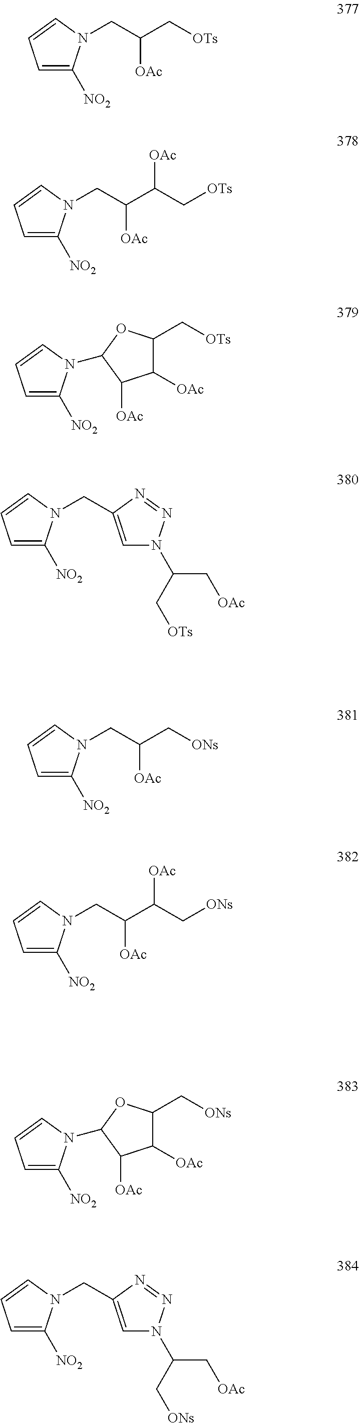

Non-radiolabelled (cold) examples of 2-nitropyrrole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy (for compounds 118 and 119 respectively):

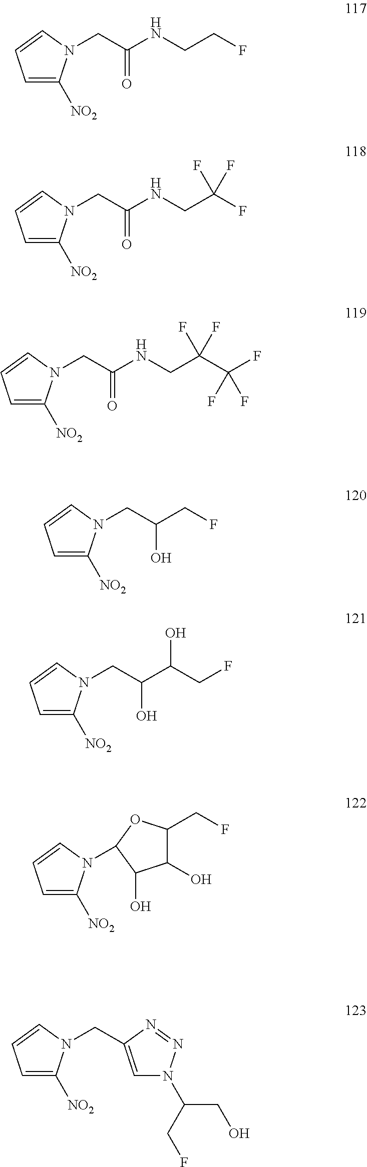

¹⁸F-labelled 2-nitropyrrole PET imaging agents for the detection of bacterial nitroreductase expression:

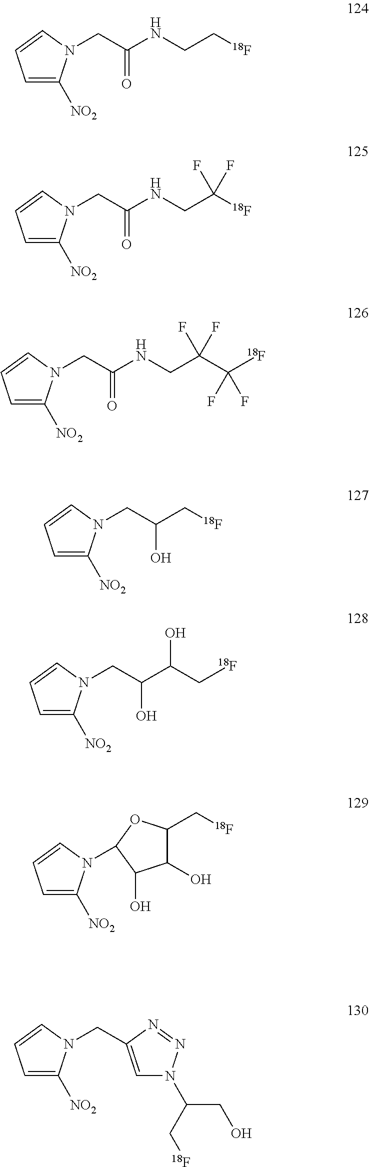

Mesylate, tosylate, nosylate and alkene radiolabelling precursors, bearing either unprotected or acetate-protected alcohol substituents, for the preparation of ¹⁸F-labelled 4-nitrophenyl PET imaging agents for the detection of bacterial nitroreductase expression:

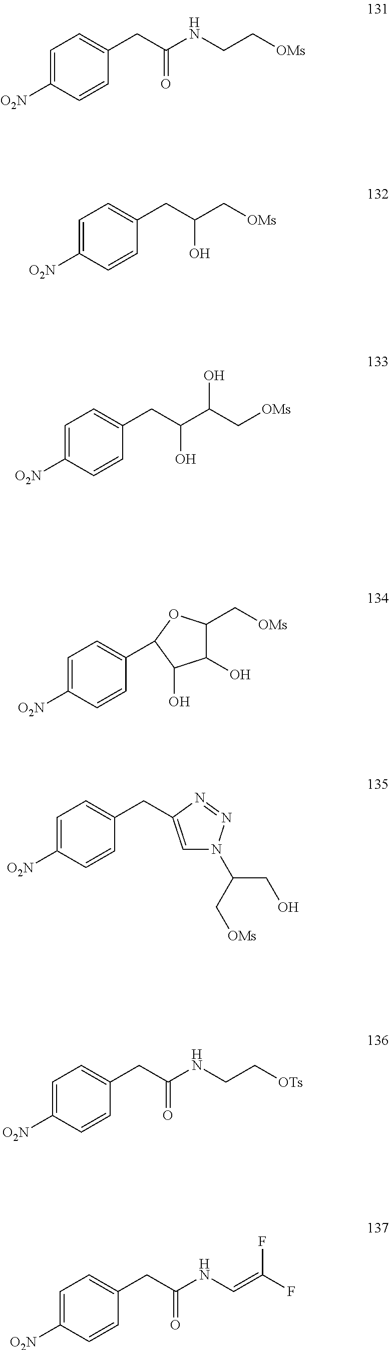

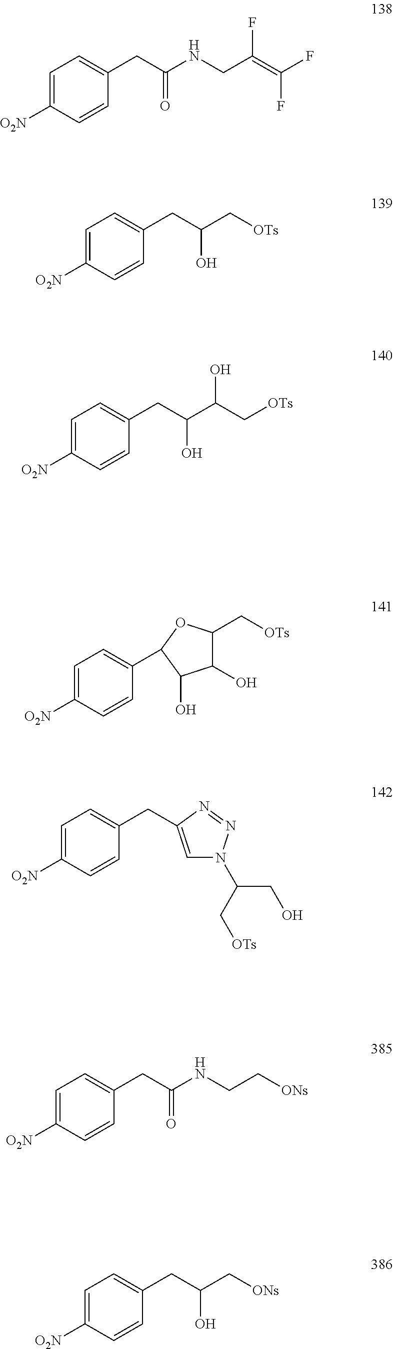

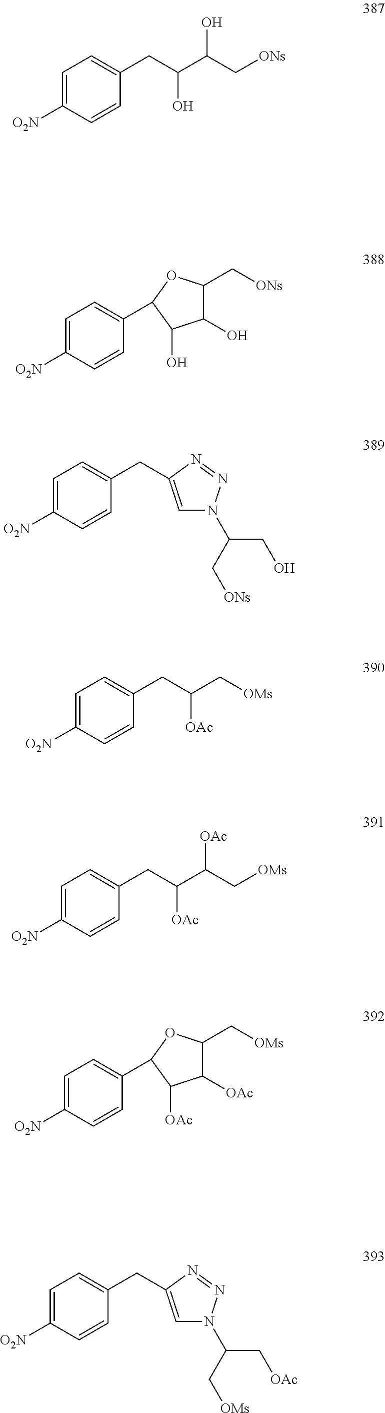

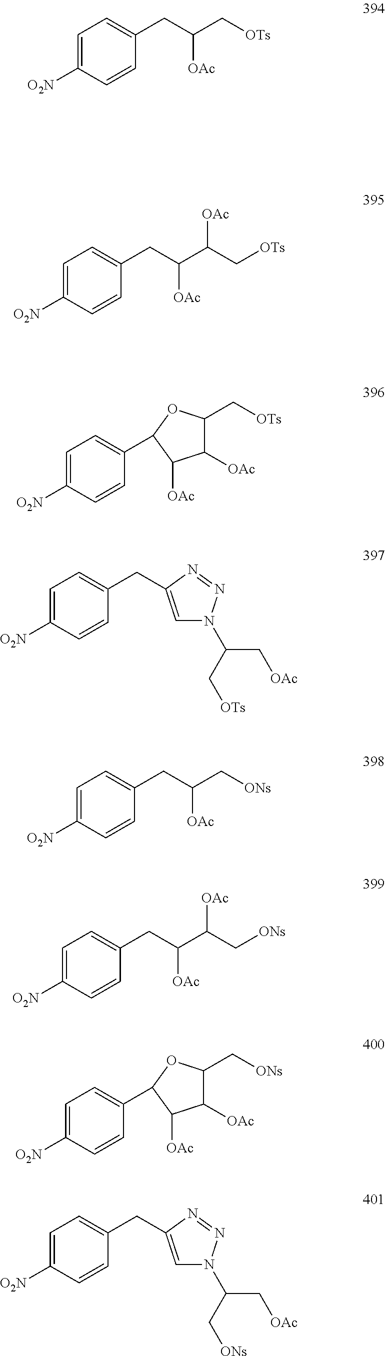

Non-radiolabelled (cold) examples of 4-nitrophenyl PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy (for compounds 144 and 145 respectively):

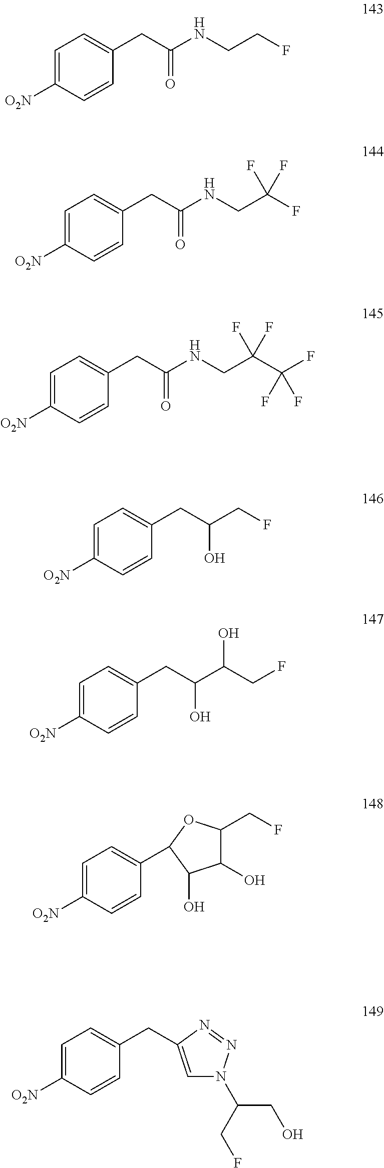

¹⁸F-labelled 4-nitrophenyl PET imaging agents for the detection of bacterial nitroreductase expression:

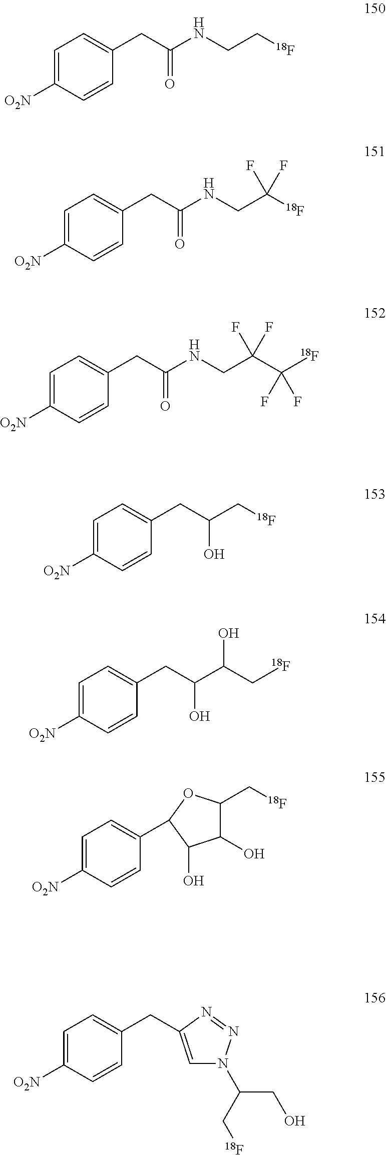

Mesylate, tosylate and nosylate radiolabelling precursors for the preparation of ¹⁸F-labelled 2-substituted-4-nitroimidazole PET imaging agents for the detection of bacterial nitroreductase expression:

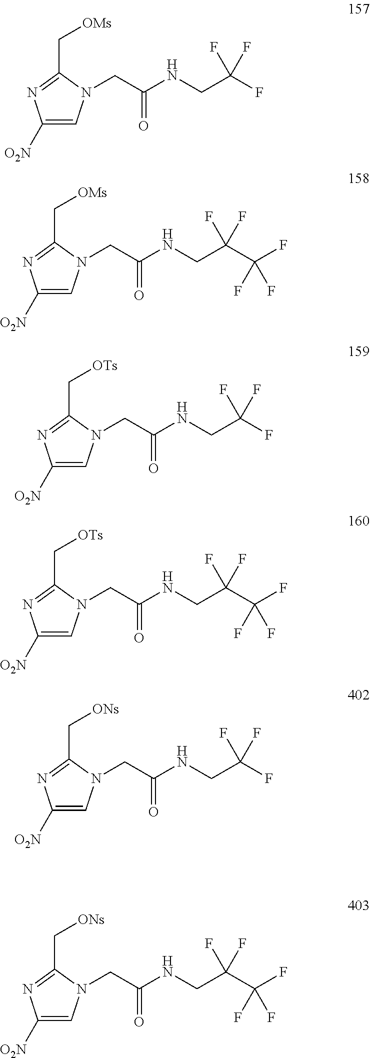

Non-radiolabelled (cold) examples of 2-substituted-4-nitroimidazole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

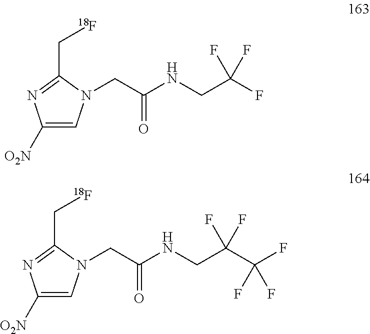 [SMILES: O=C(Cn1cc([N+](=O)[O-])nc1C[18F])NCC(F)(F)F ; O=C(Cn1cc([N+](=O)[O-])nc1C[18F])NCC(F)(F)C(F)(F)F]

¹⁸F-labelled 2-substituted-4-nitroimidazole PET imaging agents for the detection of bacterial nitroreductase expression:

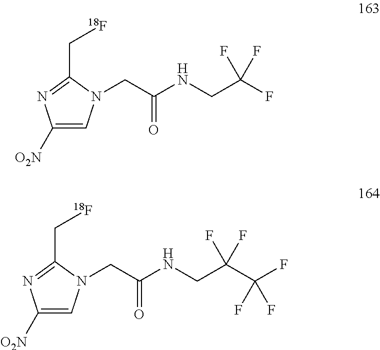 [SMILES: O=C(Cn1cc([N+](=O)[O-])nc1C[18F])NCC(F)(F)F ; O=C(Cn1cc([N+](=O)[O-])nc1C[18F])NCC(F)(F)C(F)(F)F]

Mesylate, tosylate and nosylate radiolabelling precursors for the preparation of ¹⁸F-labelled 5-substituted-4-nitroimidazole PET imaging agents for the detection of bacterial nitroreductase expression:

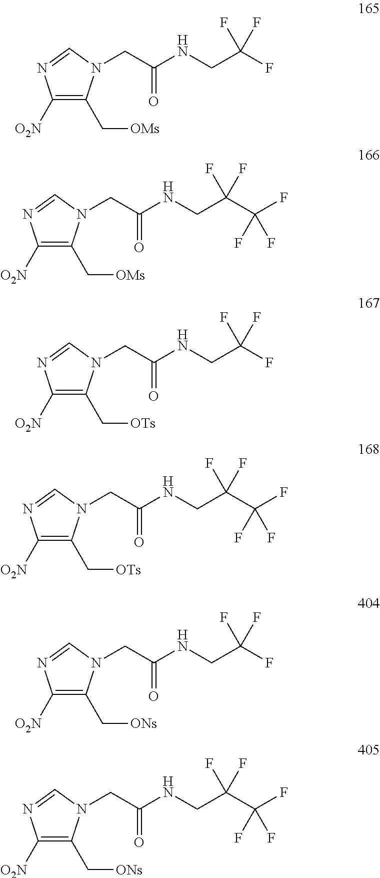

Non-radiolabelled (cold) examples of 5-substituted-4-nitroimidazole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

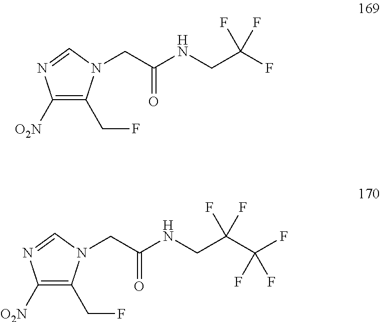 [SMILES: O=C(Cn1cnc([N+](=O)[O-])c1CF)NCC(F)(F)F ; O=C(Cn1cnc([N+](=O)[O-])c1CF)NCC(F)(F)C(F)(F)F]

¹⁸F-labelled 5-substituted-4-nitroimidazole PET imaging agents for the detection of bacterial nitroreductase expression:

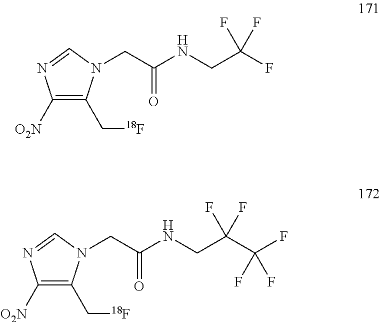 [SMILES: O=C(Cn1cnc([N+](=O)[O-])c1C[18F])NCC(F)(F)F ; O=C(Cn1cnc([N+](=O)[O-])c1C[18F])NCC(F)(F)C(F)(F)F]

Mesylate, tosylate and nosylate radiolabelling precursors for the preparation of ¹⁸F-labelled 2-substituted-5-nitroimidazole PET imaging agents for the detection of bacterial nitroreductase expression:

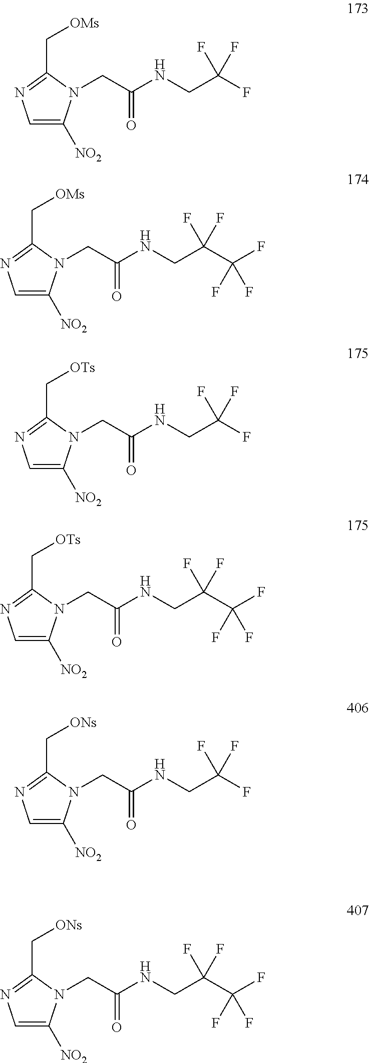

Non-radiolabelled (cold) examples of 2-substituted-5-nitroimidazole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

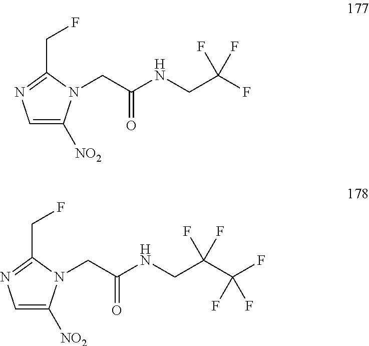 [SMILES: O=C(Cn1c([N+](=O)[O-])cnc1CF)NCC(F)(F)F ; O=C(Cn1c([N+](=O)[O-])cnc1CF)NCC(F)(F)C(F)(F)F]

¹⁸F-labelled 2-substituted-5-nitroimidazole PET imaging agents for the detection of bacterial nitroreductase expression:

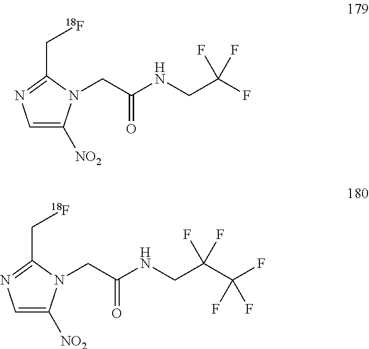 [SMILES: O=C(Cn1c([N+](=O)[O-])cnc1C[18F])NCC(F)(F)F ; O=C(Cn1c([N+](=O)[O-])cnc1C[18F])NCC(F)(F)C(F)(F)F]

Mesylate, tosylate and nosylate radiolabelling precursors for the preparation of ¹⁸F-labelled 4-substituted-5-nitroimidazole PET imaging agents for the detection of bacterial nitroreductase expression:

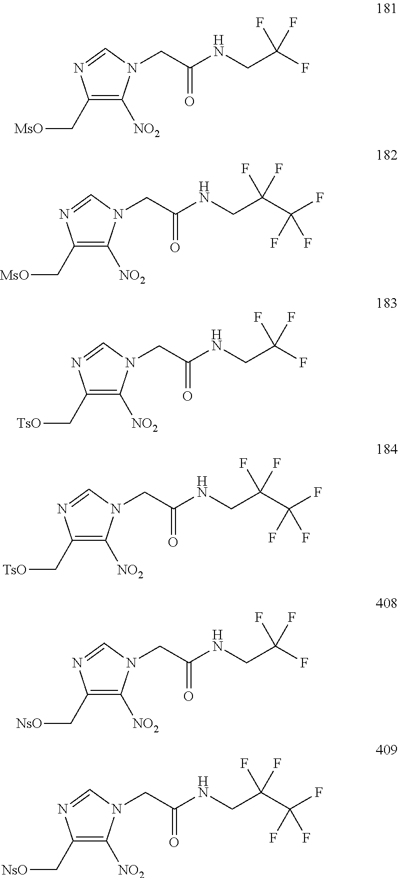

Non-radiolabelled (cold) examples of 4-substituted-5-nitroimidazole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

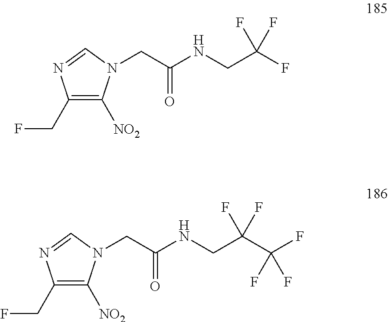 [SMILES: O=C(Cn1cnc(CF)c1[N+](=O)[O-])NCC(F)(F)F ; O=C(Cn1cnc(CF)c1[N+](=O)[O-])NCC(F)(F)C(F)(F)F]

¹⁸F-labelled 4-substituted-5-nitroimidazole PET imaging agents for the detection of bacterial nitroreductase expression:

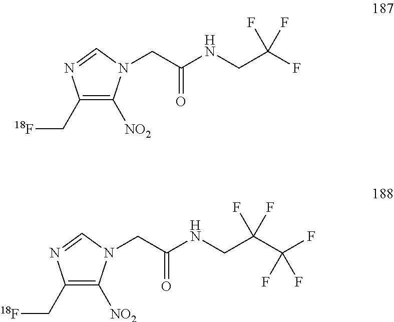 [SMILES: O=C(Cn1cnc(C[18F])c1[N+](=O)[O-])NCC(F)(F)F ; O=C(Cn1cnc(C[18F])c1[N+](=O)[O-])NCC(F)(F)C(F)(F)F]

Mesylate, tosylate and nosylate radiolabelling precursors for the preparation of ¹⁸F-labelled 2-substituted-5-nitropyrrole PET imaging agents for the detection of bacterial nitroreductase expression:

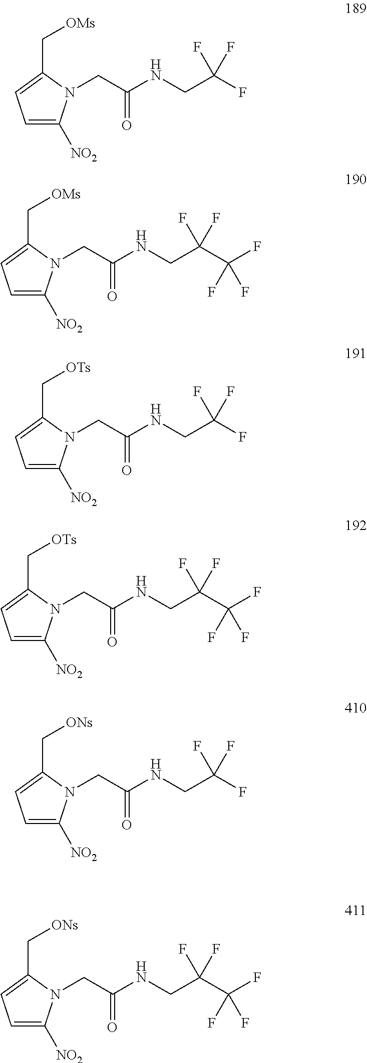

Non-radiolabelled (cold) examples of 2-substituted-5-nitropyrrole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

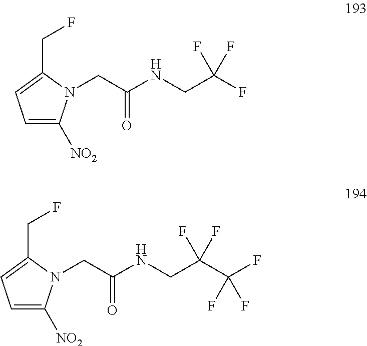 [SMILES: O=C(Cn1c(CF)ccc1[N+](=O)[O-])NCC(F)(F)F ; O=C(Cn1c(CF)ccc1[N+](=O)[O-])NCC(F)(F)C(F)(F)F]

¹⁸F-labelled 2-substituted-5-nitropyrrole PET imaging agents for the detection of bacterial nitroreductase expression:

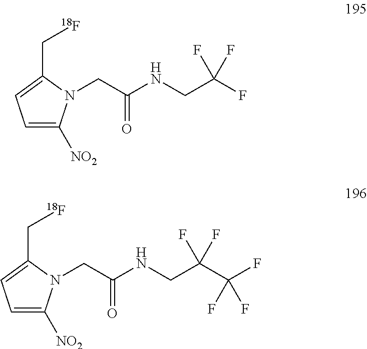 [SMILES: O=C(Cn1c(C[18F])ccc1[N+](=O)[O-])NCC(F)(F)F ; O=C(Cn1c(C[18F])ccc1[N+](=O)[O-])NCC(F)(F)C(F)(F)F]

Mesylate, tosylate and nosylate radiolabelling precursors for the preparation of ¹⁸F-labelled 3-substituted-2-nitropyrrole PET imaging agents for the detection of bacterial nitroreductase expression:

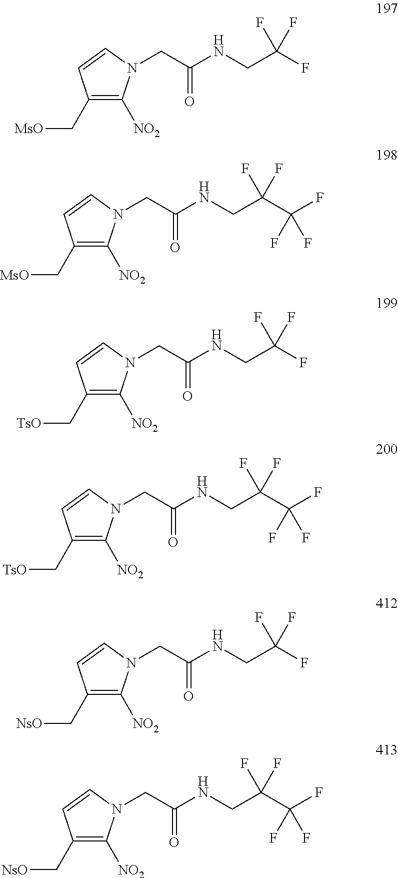

Non-radiolabelled (cold) examples of 3-substituted-2-nitropyrrole PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

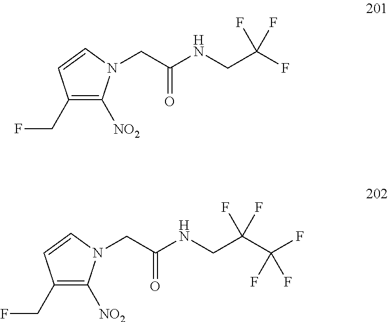 [SMILES: O=C(Cn1ccc(CF)c1[N+](=O)[O-])NCC(F)(F)F ; O=C(Cn1ccc(CF)c1[N+](=O)[O-])NCC(F)(F)C(F)(F)F]

¹⁸F-labelled 3-substituted-2-nitropyrrole PET imaging agents for the detection of bacterial nitroreductase expression:

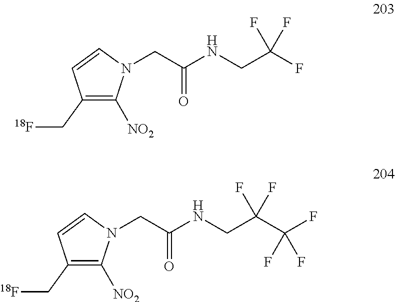 [SMILES: O=C(Cn1ccc(C[18F])c1[N+](=O)[O-])NCC(F)(F)F ; O=C(Cn1ccc(C[18F])c1[N+](=O)[O-])NCC(F)(F)C(F)(F)F]

Mesylate, tosylate and nosylate radiolabelling precursors for the preparation of ¹⁸F-labelled 1-substituted-5-nitrophenyl PET imaging agents for the detection of bacterial nitroreductase expression:

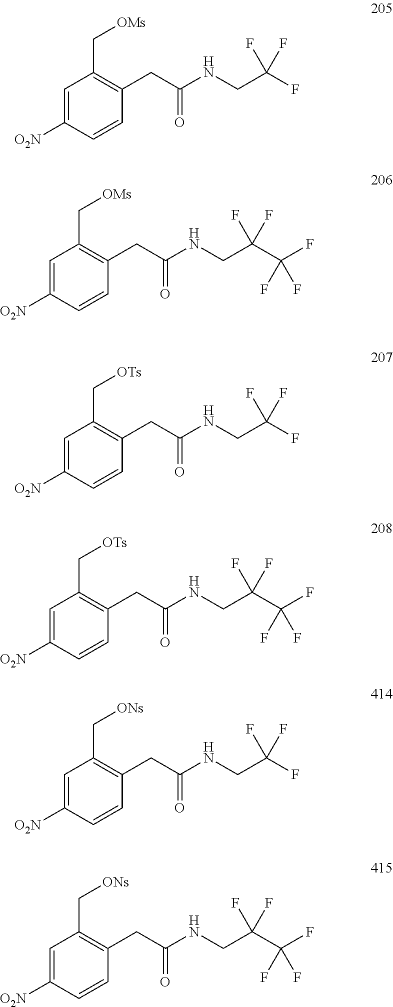

Non-radiolabelled (cold) examples of 1-substituted-5-nitrophenyl PET imaging agents for use at high dose to perform single cell ablation of bacterial nitroreductase expressing cells or biological vectors (viruses and bacteria) and for immunohistochemical detection of bacterial nitroreductase expression through antibody detection of trifluoro and pentafluoro side chain epitopes following tissue biopsy:

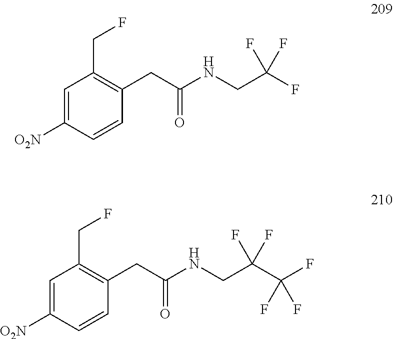 [SMILES: O=C(Cc1ccc([N+](=O)[O-])cc1CF)NCC(F)(F)F ; O=C(Cc1ccc([N+](=O)[O-])cc1CF)NCC(F)(F)C(F)(F)F]

¹⁸F-labelled 1-substituted-5-nitrophenyl PET imaging agents for the detection of bacterial nitroreductase expression:

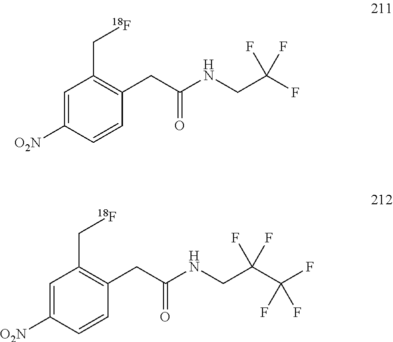 [SMILES: O=C(Cc1ccc([N+](=O)[O-])cc1C[18F])NCC(F)(F)F ; O=C(Cc1ccc([N+](=O)[O-])cc1C[18F])NCC(F)(F)C(F)(F)F]

### Dual Use as a Radiolabelled Imaging Probe and a “Cold” Immunohistochemistry Probe

In a further embodiment the invention provides compounds of general formula I or V as defined above wherein Y is selected from IIIb, IIIc or IIIh and R is selected from CH₂F or CH₂¹⁸F. These compounds have utility as imaging agents that act as a substrate for an antibody specific to the compound. Such compounds have particular utility in immunohistochemical imaging.

In this embodiment of the invention, the compound has dual utility as a) a radiolabelled imaging probe for PET/SPECT imaging and b) a non-radiolabelled probe for immunohistochemical analysis. The radiolabelled probe and the non-radiolabelled “cold” probe are effectively the same compound and only differ in the isotopic form of one of the nuclides i.e. one contains a radionuclide while the other has a non-radioactive nuclide. Since the compounds are effectively the same when considering chemical and pharmacokinetic properties, the same compound can be used to image NTR metabolism using two independent methods thereby providing for cross-validation of NTR expression/metabolism.

Such imaging agents have particular utility in correlating PET imaging with immunohistochemical analysis of the extent of vectors and/or biological agents introduced as part of GDEPT or gene therapy strategies.

The immunohistochemical analysis is carried out using antibodies generated to recognise the particular structural conformation of the Y side chains. In a particular embodiment, the compound of the invention is administered to the subject in a relatively high dose (compared to the dose used for radiolabelled imaging), then a tissue biopsy is taken and stained with an antibody specific to the compound structure. Imaging of the bound antibody is used to determine the extent and concentration of the NTR expressing cell and/or biological agent.

A compound of this aspect also has a further advantage over known compounds (for example EF3, EF5 and pimonidazole) as the radiolabelled imaging probe can be prepared by safer and more convenient methods. In order to prepare radiolabelled EF3 and EF5, ¹⁸F₂ gas is required and the reaction normally proceeds by addition of the radiolabelled fluorine to a double-bond. In contrast, in a particular embodiment of the present invention, the compound is prepared using the safer and more convenient Na¹⁸F and proceeds via fluoride displacement of a mesylate, tosylate or nosylate attached to the R group of the precursor compound. The Y side chain in this embodiment contains “cold” fluorine nuclides (where relevant) which can be detected using specific antibodies (for example EF3, EF5 or piminidazole antibodies) thereby providing a dual-use compound detectable by different imaging methods. The inventors have demonstrated the utility of compounds of the invention in this dual-use imaging application (i.e. PET and IHC) in the examples using compounds 67 and 93 with an EF5 antibody (see FIG. 7). In a further embodiment of this aspect, the compound has utility as a single cell ablator compound.

### Synthesis of Radiolabelled Imaging Probes and “Cold” Compounds

The invention provides precursor compounds of general formula I or V and in particular embodiments, Y is selected from formula IVa to IVg. ¹⁸F-labelled PET imaging agents are made from appropriate precursor molecules such as, but not limited to, alkenes, mesylates, tosylates, nosylates, trifluoromethanesulfonates, chlorides, bromides and iodides by reaction with ¹⁸F-labelled fluorine gas (for alkene precursors) or ¹⁸F-labelled fluoride salts such as Na¹⁸F, OF and Bu₄¹⁸F (for mesylates, tosylates, nosylates, trifluoromethanesulfonates, chlorides, bromides and iodides) using methods familiar to one skilled in the art. The corresponding “cold” compounds are prepared in a similar way but using non-radioactive isotopes.

The below figure uses labelling of precursor molecules for the production of [¹⁸F]-EF3, [¹⁸F]-EF5, [¹⁸F]-MISO and [¹⁸F]-HX4 as specific examples of this approach. Similar techniques may be used to prepare compounds of use in the invention from their precursor compounds.

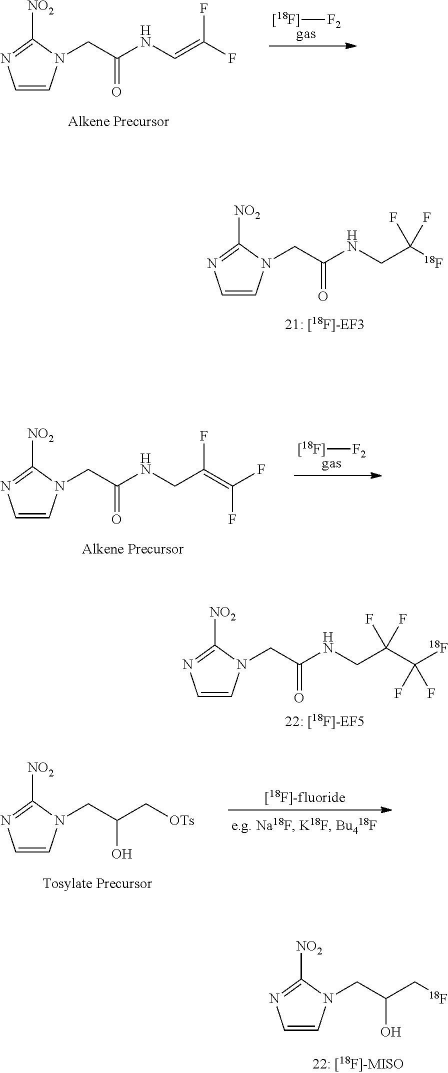

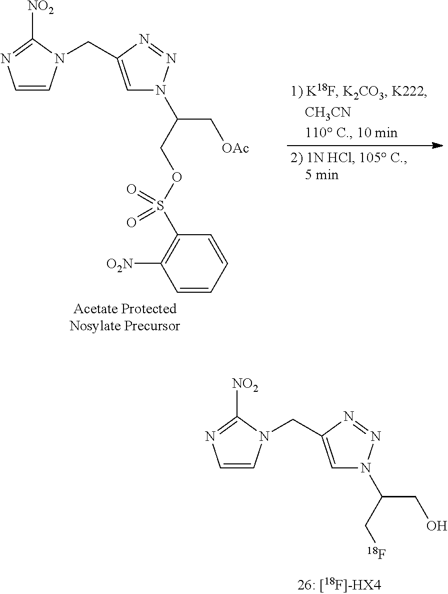 [SMILES: CC(=O)OCC(COS(=O)(=O)c1ccccc1[N+](=O)[O-])n1cc(Cn2ccnc2[N+](=O)[O-])nn1 ; O=[N+]([O-])c1nccn1Cc1cn(C(CO)C[18F])nn1]

In further embodiments, the invention provides a compound of general formula I or V where Y is selected from IIg or IIIg. These embodiments have particularly utility as imaging agents because such compounds are believed to have the capacity to be quickly removed from the body during and after administration.

In a further aspect, the invention provides a method of treatment or diagnosis of a disease using a compound of general formula I or V as defined above wherein the disease is selected from the group consisting of cancer, Parkinson's disease, Alzheimer's disease, stroke, heart disease, rheumatological diseases and a disease treated by stem-cell transplantation.

In a further aspect, the invention provides the use of a compound of general formula I or V as defined above in the manufacture of a medicament for the treatment of a disease selected from the group consisting of cancer, Parkinson's disease, Alzheimer's disease, stroke, heart disease, rheumatological diseases and a disease treated by stem-cell transplantation.

In a further aspect, the invention provides a compound of general formula I or V as defined above for use in the treatment of a disease selected from the group consisting of cancer, Parkinson's disease, Alzheimer's disease, stroke, heart disease, rheumatological diseases and a disease treated by stem-cell transplantation.

In a further aspect, the invention provides a composition comprising a compound of general formula I or V as defined above and a pharmaceutically acceptable diluent, excipient, carrier or adjuvant.

Compounds of use in the invention may be introduced to a subject in any way. Typically, the compound will be introduced as a compositions or medicament by standard methods of administration. The compositions or medicaments of the invention may include a pharmaceutically acceptable diluent, carrier, excipient and/or adjuvant of any of the foregoing. The choice of diluent, carrier, excipient and/or adjuvant can depend upon, among other factors, the desired mode of administration. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The compositions or medicaments can additionally include lubricating agents such as talc, magnesium stearate, and mineral oil, wetting agents, emulsifying and suspending agents, preserving agents such as methyl- and propylhydroxy-benzoates, sweetening agents, pH adjusting and buffering agents, toxicity adjusting agents, flavoring agents, and the like. The compositions or medicaments can be formulated so as to provide quick, sustained or delayed release of the active ingredient after administration to the patient by employing procedures known in the art. A composition or medicament can be formulated in unit dosage form, each dosage comprising a physically discrete unit suitable as a unitary dosage for humans and other mammals, each unit containing a predetermined quantity of active material calculated to produce the desired therapeutic effect, in association with a suitable pharmaceutical excipient, diluent, carrier and/or adjuvant.

In a further aspect, the invention provides a kit for evaluation of in vivo distribution of a nitroreductase-expressing cell and/or biological agent comprising a compound of general formula I or V as defined above.

In a further aspect, the invention provides a kit comprising a one or more of:
a. a radiolabelled compound according to formula 104:

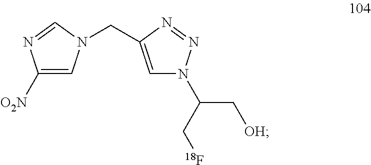 [SMILES: O=[N+]([O-])c1cn(Cc2cn(C(CO)C[18F])nn2)cn1]

b. a precursor compound according to formula 367:

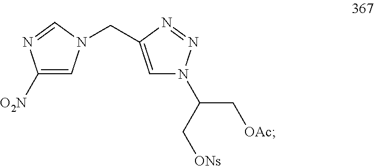

and/or
c. a compound according to formula 97:

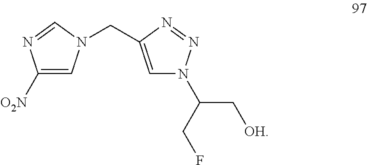 [SMILES: O=[N+]([O-])c1cn(Cc2cn(C(CO)CF)nn2)cn1]

In a particular embodiment, the kit is used in conjunction with a nitroreductase enzyme expressed by a wild type or mutant variant of E coli NfsA.

In a further aspect, the invention provides a kit for the control of a cell and/or a biological agent comprising a compound of general formula I or V as defined above.

### Synthesis of Compounds of the Invention

The invention provides a method of synthesis of a non-precursor compound of general formula I or V as defined above. In particular embodiments, the non-precursor compound is synthesised from a precursor compound of general formula I or V as defined above.

In a particular embodiment, the method comprises a) a fluoride displacement of a mesylate, tosylate or nosylate followed by in situ deprotection of any protecting groups where necessary or b) a fluorine gas addition to a double bond or c) amide coupling of fluorinated amine intermediates with their acid counterparts to provide “cold” fluorine containing compounds or d) click coupling of azide intermediates with alkynes to provide triazole derivatives.

In a particular embodiment, the compound comprises compound 67, the ¹⁸F-labelled analogue 74 and its alkene radiolabelling precursor 60 and the method comprises a Swern oxidation as described below:

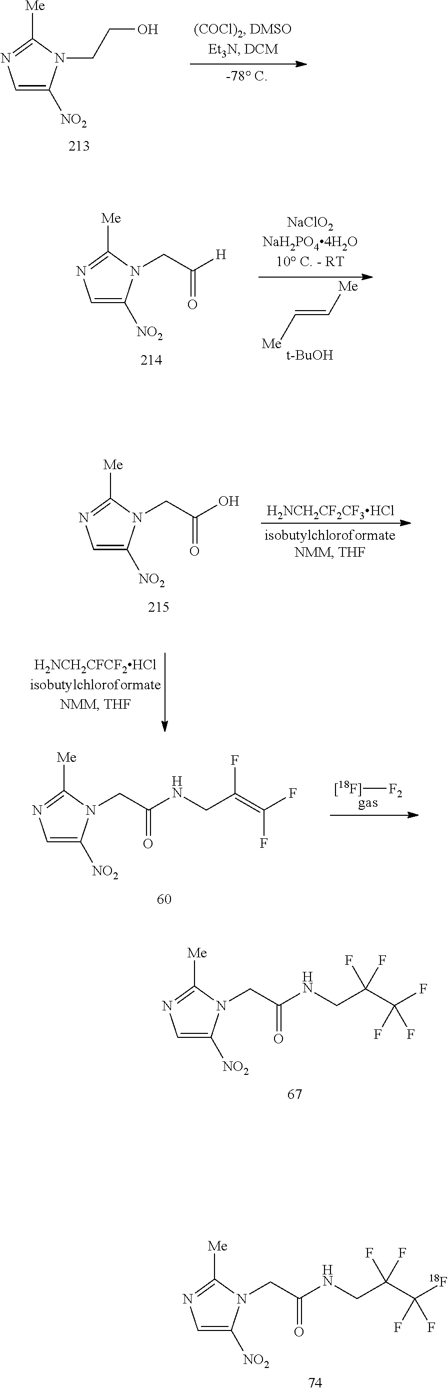

Swern oxidation of commercially available metronidazole (213) provided the aldehyde 214 which was subsequently further oxidised with sodium chlorite to give the acid 215. Isobutylchloroformate-mediated amide coupling of acid 215 with the free base of 2,2,3,3,3-pentafluoropropylamine hydrochloride then gave the desired compound 67. The direct ¹⁸F-labelled analogue 74 can similarly be prepared from isobutylchloroformate-mediated amide coupling of acid 215 with the free base of 2,3,3-trifluoroprop-2-en-1-amine hydrochloride to give the precursor 60, which is in turn reacted with ¹⁸F-fluorine gas.

In a particular embodiment, the compound comprises compound 93, the ¹⁸F-labelled analogue 100 and its alkene radiolabelling precursor 86 and the method comprises alkylation as described below:

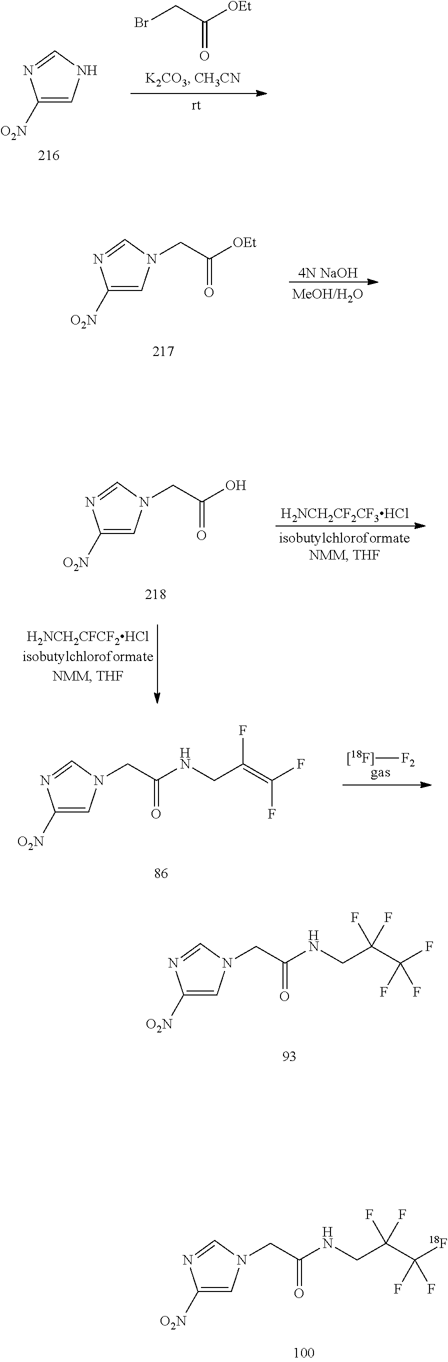

Potassium carbonated mediated alkylation of 4-nitroimidazole (216) with ethyl 2-bromoacetate gave ester 217 which was subsequently hydrolysed to acid 218. Isobutylchloroformate-mediated amide coupling of acid 218 with the free base of 2,2,3,3,3-pentafluoropropylamine hydrochloride then gave the desired compound 93. The direct ¹⁸F-labelled analogue 100 can similarly be prepared from isobutylchloroformate-mediated amide coupling of acid 218 with the free base of 2,3,3-trifluoroprop-2-en-1-amine hydrochloride to give the precursor 86, which is in turn reacted with ¹⁸F-fluorine gas.

In a particular embodiment, the compound comprises compound 119, the ¹⁸F-labelled analogue 126 and its alkene radiolabelling precursor 112 and the method comprises alkylation as described below:

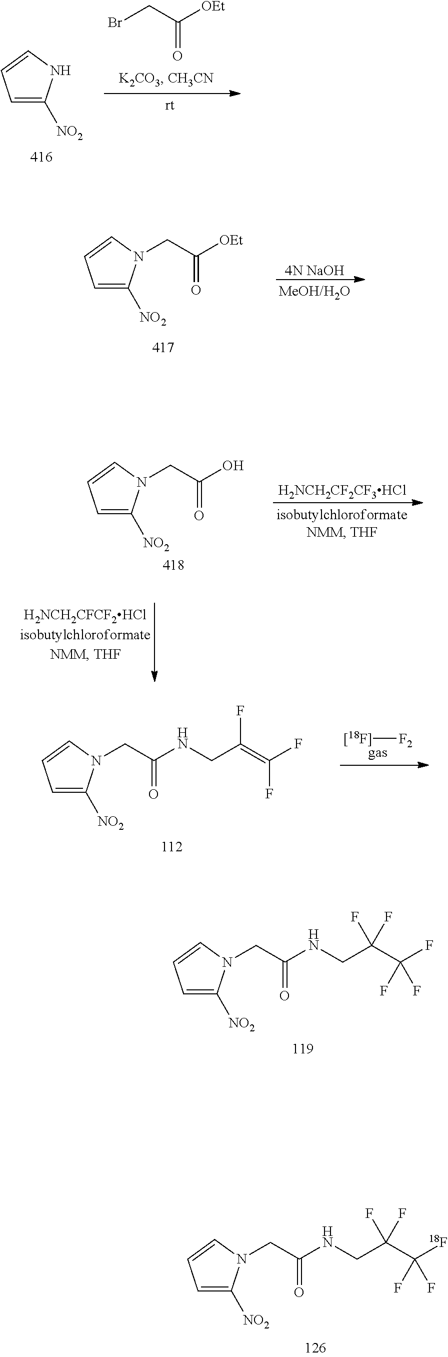

Potassium carbonated mediated alkylation of 2-nitropyrrole (416) with ethyl 2-bromoacetate to give ester 217 which can subsequently be hydrolysed to acid 418. Isobutylchloroformate-mediated amide coupling of acid 418 with the free base of 2,2,3,3,3-pentafluoropropylamine hydrochloride will then give the desired compound 119. The direct ¹⁸F-labelled analogue 126 can similarly be prepared from isobutylchloroformate-mediated amide coupling of acid 418 with the free base of 2,3,3-trifluoroprop-2-en-1-amine hydrochloride to give the precursor 112, which is in turn reacted with ¹⁸F-fluorine gas.

In a particular embodiment, the compound comprises compound 145, the ¹⁸F-labelled analogue 152 and it's alkene radiolabelling precursor 138 and the method comprises an amide coupling as described below:

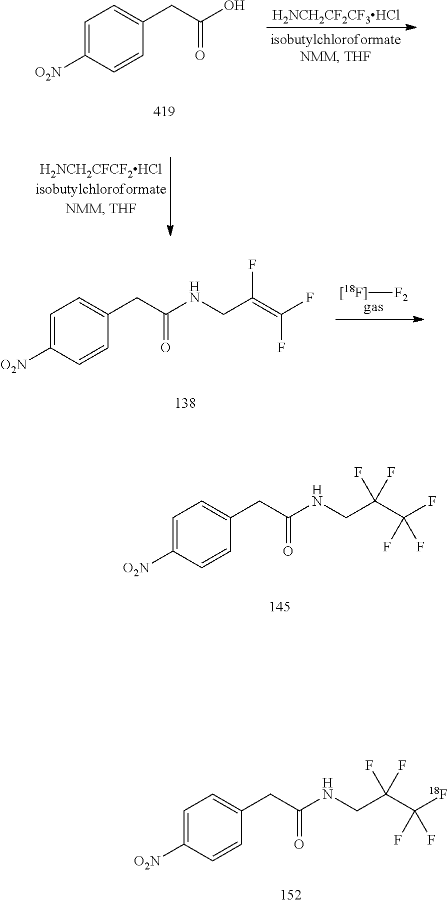

Isobutylchloroformate-mediated amide coupling of 2-(4-nitrophenyl)acetic acid 419 with the free base of 2,2,3,3,3-pentafluoropropylamine hydrochloride will give the desired compound 145. The direct ¹⁸F-labelled analogue 152 can similarly be prepared from isobutylchloroformate-mediated amide coupling of acid 419 with the free base of 2,3,3-trifluoroprop-2-en-1-amine hydrochloride to give the precursor 138, which is in turn reacted with ¹⁸F-fluorine gas.

In a particular embodiment, the compound comprises compound 71, the ¹⁸F-labelled analogue 78 and it's acetate-protected nosylate radiolabelling precursor 350 and the method comprises a Swern oxidation as described below:

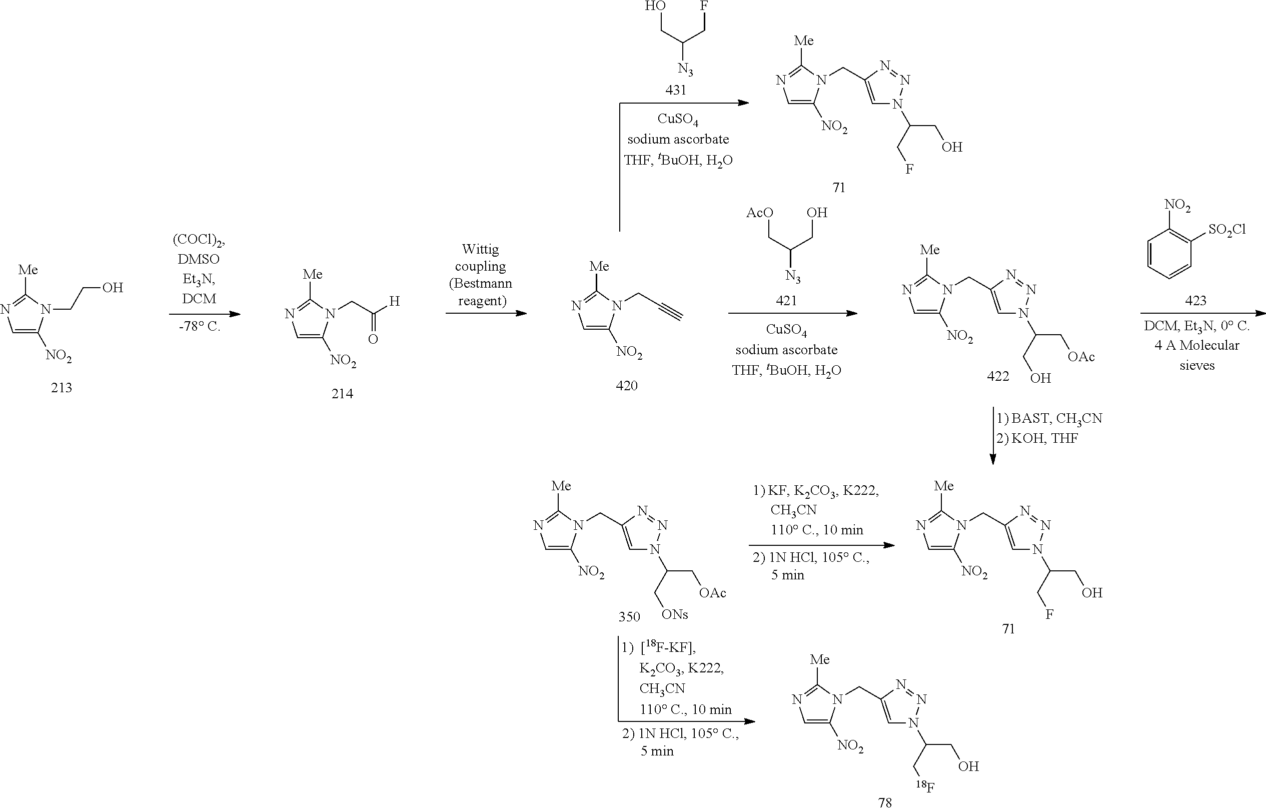

Swern oxidation of commercially available metronidazole (213) provided the aldehyde 214 which can subsequently undergo Wittig coupling with Bestmann-Ohira reagent [Synthetic Communications, 1989, 19(3&4), 561-564] to provide the alkyne 420. Click coupling of this alkyne with the known azide 421 [WO2008/124651A2 PCT/US2008/059505] will afford the triazole 422, which can be fluorinated directly with BAST and then deprotected to give the “cold” fluorinated derivative 71. Alternately, reaction with nosyl chloride (423) will give the acetate-protected nosylate radiolabelling precursor 350. Reaction of this with “cold” potassium fluoride, followed by acid mediated in situ protection of the acetate group will provide compound 71, similarly reaction with [¹⁸F] potassium fluoride followed by acetate deprotection will give the ¹⁸F-labelled PET agent 78. The “cold” fluorinated derivative 71 can alternately be prepared by click coupling of alkyne 420 with the known azide 431 [WO2008/124651 A2 PCT/US2008/059505].

In a particular embodiment, the compound comprises compound 97, the ¹⁸F-labelled analogue 104 and it's acetate-protected nosylate radiolabelling precursor 367 and the method comprises an alkylation as described below:

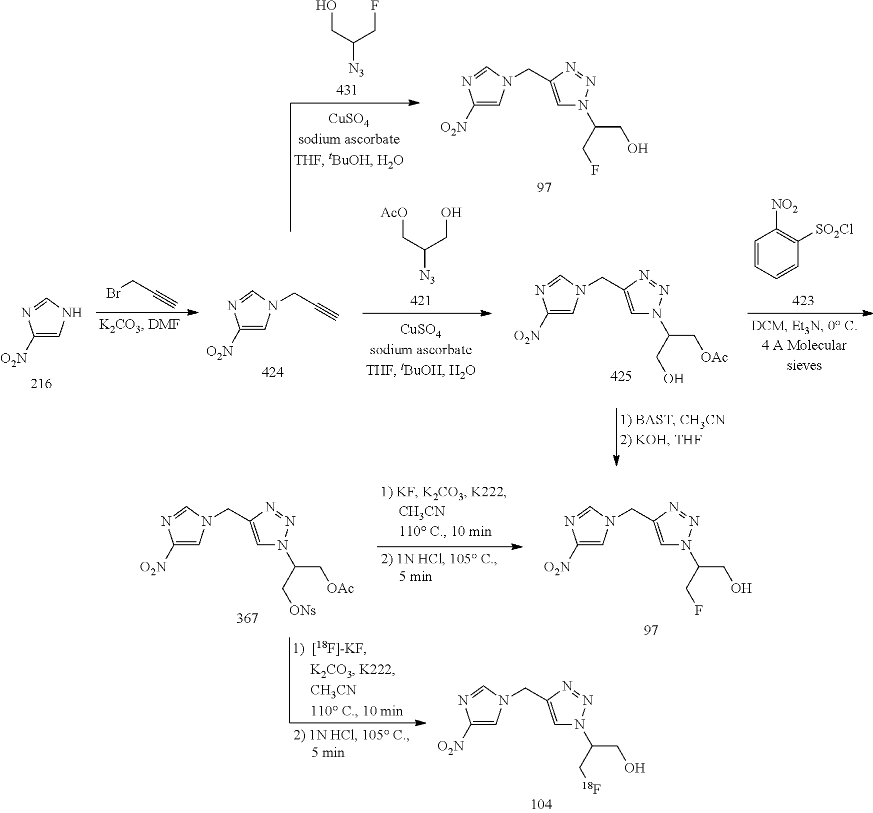

Potassium carbonate mediated alkylation of commercially available 4-nitroimidazole (216) provided the alkyne 424. Click coupling of this alkyne with the known azide 421 [WO2008/124651A2 PCT/US2008/059505] then gave the triazole 425, which can be fluorinated directly with BAST and then deprotected to give the “cold” fluorinated derivative 97. Alternately, alcohol 425 was reacted with nosyl chloride (423) to give the acetate-protected nosylate radiolabelling precursor 367. Reaction of this with “cold” potassium fluoride, followed by acid mediated in situ protection of the acetate group gave compound 97, similarly reaction with [¹⁸F] potassium fluoride followed by acetate deprotection will give the ¹⁸F-labelled PET agent 104. The “cold” fluorinated derivative 97 can alternately be prepared by click coupling of alkyne 424 with the known azide 431 [WO2008/124651A2 PCT/US2008/059505].

In a particular embodiment, the compound comprises compound 123, the ¹⁸F-labelled analogue 130 and it's acetate-protected nosylate radiolabelling precursor 384 and the method comprises an alkylation as described below:

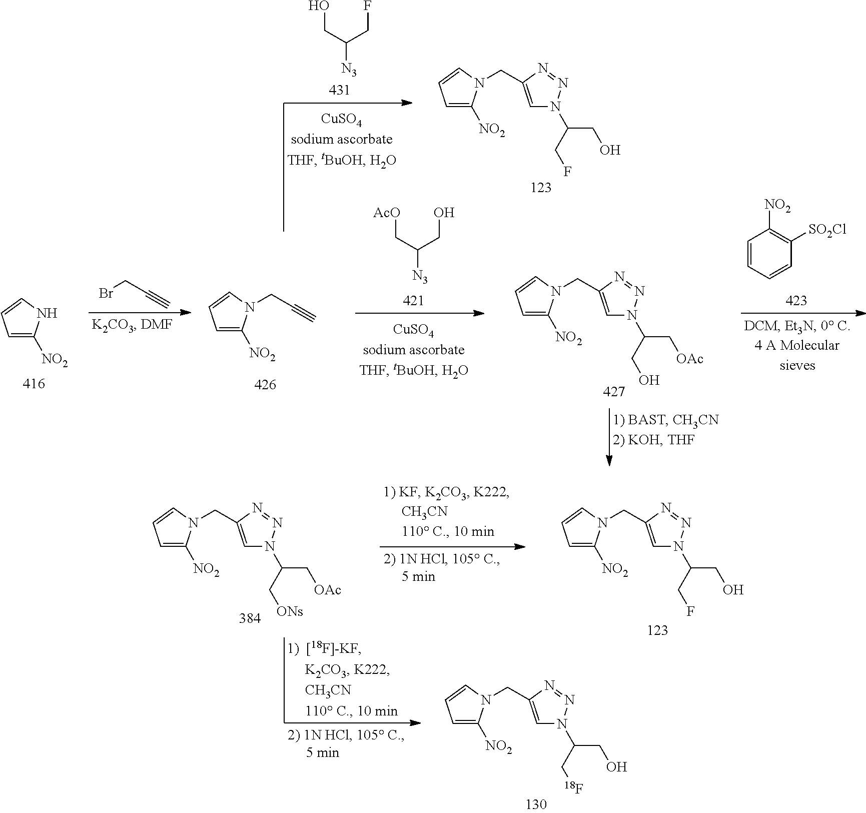

Potassium carbonate mediated alkylation of commercially available 2-nitropyrrole (416) will provide the alkyne 426. Click coupling of this alkyne with the known azide 421 [WO2008/124651A2 PCT/US2008/059505] will then give the triazole 427, which can be fluorinated directly with BAST and then deprotected to give the “cold” fluorinated derivative 123. Alternately, alcohol 425 can be reacted with nosyl chloride (423) to give the acetate-protected nosylate radiolabelling precursor 367. Reaction of this with “cold” potassium fluoride, followed by acid mediated in situ protection of the acetate group will also afford compound 123, similarly reaction with [¹⁸F] potassium fluoride followed by acetate deprotection will then give the ¹⁸F-labelled PET agent 130. The “cold” fluorinated derivative 123 can alternately be prepared by click coupling of alkyne 426 with the known azide 431 [WO2008/124651 A2 PCT/US2008/059505].

In a particular embodiment, the compound comprises compound 149, the ¹⁸F-labelled analogue 156 and it's acetate-protected nosylate radiolabelling precursor 401 and the method comprises a Wittig coupling followed by a click coupling as described below:

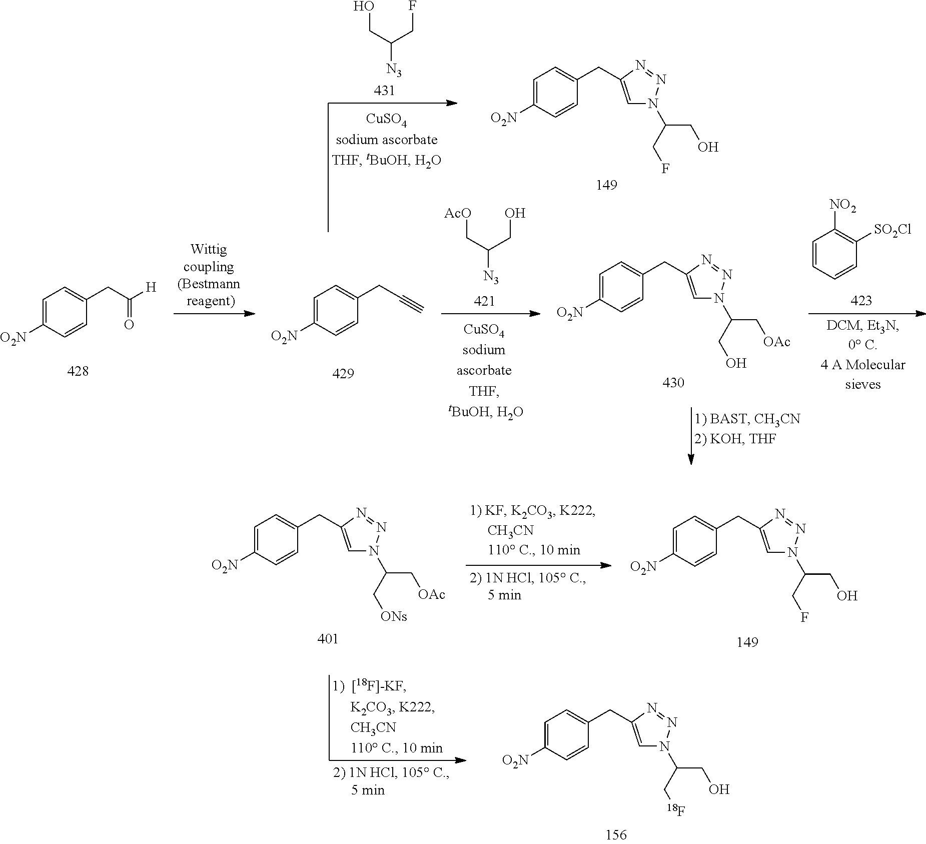

Wittig coupling of 2-(4-nitrophenyl)acetaldehyde (428) with Bestmann-Ohira reagent [Synthetic Communications, 1989, 19(3&4), 561-564] will provide the alkyne 429. Click coupling of this alkyne with the known azide 421 [WO2008/124651A2 PCT/US2008/059505] will then give the triazole 430, which can be fluorinated directly with BAST and then deprotected to give the “cold” fluorinated derivative 149. Alternately, alcohol 430 can be reacted with nosyl chloride (423) to give the acetate-protected nosylate radiolabelling precursor 401. Reaction of this with “cold” potassium fluoride, followed by acid mediated in situ protection of the acetate group will also afford compound 149, similarly reaction with [¹⁸F] potassium fluoride followed by acetate deprotection will then give the ¹⁸F-labelled PET agent 156. The “cold” fluorinated derivative 149 can alternately be prepared by click coupling of alkyne 429 with the known azide 431 [WO2008/124651A2 PCT/US2008/059505].

It will be appreciated that the compounds of the invention may occur in different geometric and enantiomeric forms, and that both pure forms and mixtures of these compounds are included.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may be said broadly to consist in the parts, elements and features referred to or indicated in the specification, individually or collectively, in any or all combinations of two or more of said parts, elements or features.

Wherein the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the scope of the invention.

### EXAMPLES

### Example 1

### Experimental for the synthesis of 2-methyl-5-nitroimidazol-1-N-2,2,3,3,3-pentafluoropropyl acetamide (67)

Swern oxidation of metronidazole (213) according to the reported method (WO 2008/008480 PCT/US2007/015970) provided crude 2-(2-methyl-5-nitro-1H-imidazol-1-yl)acetaldehyde 214 (3.08 g, 61%) which was used directly.

A solution of NaClO₂ (16.47 g, 182.10 mmol) in water (65 mL) was added dropwise to a stirred mixture of 2-(2-methyl-5-nitro-1H-imidazol-1-yl)acetaldehyde 214 (3.08 g, 18.21 mmol) and 2-methyl-2-butene (48.23 mL, 455.24 mmol) in tert-butanol (260 mL), and NaH₂PO₄.4H₂O (19.89 g, 127.47 mmol) in water (65 mL). The mixture was stirred overnight then acidified with HCl (10%, 200 mL). The aqueous phase was then extracted with EtOAc (×3) and the combined organic layers were washed with water and brine, dried, and concentrated under reduced pressure. The residue was triturated with petroleum ether to give 2-(2-methyl-5-nitro-1H-imidazol-1-yl)acetic acid 215 as a pale yellow gum (426 mg, 13%). ¹H NMR [(CD₃)₂SO] δ (OH not seen) 8.06 (s, 1H), 5.08 (s, 2H), 2.42 (s, 3H). LRMS (APCI) calcd. for C₆H₈N₃O₄ (M+1) m/z 186.15. found 186.60; calcd. for C₆H₆N₃O₄ (M−1) m/z 184.13. found 184.50.

Isobutyl chloroformate (354 mg, 2.59 mmol) was added to a stirred solution of 2-(2-methyl-5-nitro-1H-imidazol-1-yl)acetic acid 215 (400 mg, 2.16 mmol) and N-methylmorpholine (570 μL, 5.18 mmol) in THF (80 mL) at 0° C. and under N₂. The mixture was stirred at 0° C. for 1 h and then solid 2,2,3,3,3-pentafluoropropylamine hydrochloride was added portionwise. The reaction mixture was then allowed to warm to room temperature with further stirring overnight. The mixture was evaporated to dryness and the residue was dissolved in EtOAc, washed with water and brine, dried and concentrated under reduced pressure. The crude product was purified by column chromatography on silica gel eluting with dichloromethane:MeOH (16:1) to give 2-(2-methyl-5-nitro-1H-imidazol-1-yl)-N-(2,2,3,3,3-pentafluoropropyl)acetamide 67 as a pale yellow solid (112 mg, 16%), mp 149-151° C.; ¹H NMR [(CD₃)₂SO] δ 9.04 (t, J=6.2 Hz, 1H), 8.04 (s, 1H), 5.08 (s, 2H), 4.02 (sextet, J=6.3 Hz, 2H), 2.37 (s, 3H). Anal. Calcd. for C₉H₉F₅N₄O₃: C, 34.19; H, 2.87; N, 17.72%. Found: C, 34.54; H, 2.93; N, 17.36%.

### Example 1.1

### Experimental for the synthesis of 3-fluoro-2-(4-((4-nitro-1H-imidazol-1-yl)methyl)-1H-1,2,3-triazol-1-yl)propan-1-ol (97)

Potassium carbonate mediated alkylation of 4-nitroimidazole 216 with 3-bromoprop-1-yne according to the procedure described by Rao et al [Journal of Chemical Synopses, 1993, 12, 506-507] gave 4-nitro-1-(prop-2-yn-1-yl)-1H-imidazole 424.

A solution of 4-nitro-1-(prop-2-yn-1-yl)-1H-imidazole 424 (191.6 mg, 1.27 mmol) and 2-azido-3-fluoropropan-1-ol 431 (prepared according to the procedure described in WO2008/124651A2 PCT/US2008/059505) (212.5 mg, 1.78 mmol) in THF:t-BuOH:H₂O (6.5 mL, 2.5:2.5:1.5) was treated with CuSO₄.H₂O (23 mg, 0.09 mmol) and sodium ascorbate (53 mg, 0.27 mmol). The reaction mixture was vigorously stirred overnight at the room temperature then diluted with CH₂Cl₂ and evaporated to dryness. The residue was purified by flash chromatography on silica gel eluting with EtoAc:Hexane followed by CH₂Cl₂:MeOH (9:1) to provide 3-fluoro-2-(4-((4-nitro-1H-imidazol-1-yl)methyl)-1H-1,2,3-triazol-1-yl)propan-1-ol 97 (188.4 mg, 55%) as a white solid, m.p. 126-129° C. ¹HNMR [(CD₃)₂SO] δ 8.42 (d, J=1.4 Hz, 1H), 8.29 (br, s, 1H), 7.96 (d, J=1.4 Hz, 1H), 5.44 (s, 2H), 5.25 (t, J=5.5 Hz, 1H), 5.01-4.89 (m, 2H), 4.83-4.75 (m, 1H), 3.82 (t, J=5.5 Hz, 2H). Anal. Calcd for C₉H₁₁FN₆O₃: C, 40.00; H, 4.10; N, 31.10%. found: C, 40.27; H, 4.07; N, 31.20%. HPLC Purity 95%.

### Example 1.2

### Experimental for the synthesis of 2-(4-((4-nitro-1H-imidazole-1-yl)methyl)-1H-1,2,3-triazol-1-yl)-3-(((2-nitrophenyl)sulfonyl)oxy) propyl acetate (367)

A solution of 4-nitro-1-(prop-2-yn-1-yl)-1H-imidazole 424 (950 mg, 6.29 mmol) and 2-azido-3-hydroxypropyl acetate 421 (prepared according to the procedure described in WO2008/124651A2 PCT/US2008/059505) (1 g, 6.28 mmol) in THF:t-BuOH:H₂O (21 mL, 1:1:1) was treated with CuSO₄.H₂O (81.2 mg, 0.33 mmol) and sodium ascorbate (126 mg, 0.64 mmol). The reaction mixture was vigorously stirred overnight at the room temperature then diluted with CH₂Cl₂ and evaporated to dryness. The residue was purified by flash chromatography on silica gel eluting with EtoAc:Hexane followed by CH₂Cl₂:MeOH (9:1) to provide triazole 425 (1.1 g, 56%) as a white solid, m.p. 118-121° C. ¹HNMR [(CD₃)₂SO] δ 8.40 (d, J=1.4 Hz, 1H), 8.29 (br, s, 1H), 7.94 (d, J=1.5 Hz, 1H), 5.43 (s, 2H), 5.23 (br, s, 1H), 4.90-4.84 (m, 1H), 4.47-4.39 (m, 2H), 3.82-3.81 (m, 2H), 1.93 (s, 3H). HRMS (ESI) Calc. for C₁₁H₁₄N₆NaO₅ [M+Na]⁺ m/z 333.0918. found 333.0919.

To a solution of triazole 425 (862 mg, 2.78 mmol) in anhydrous CH₂Cl₂ (38 mL) and acetonitrile (19 mL) was added 4 Å molecular sieves (750 mg) and AR grade Et₃N (774 μL, 5.56 mmol) at 0° C. The reaction mixture was stirred for 1 h at 0° C. then treated with 2-nitrobenzenesulfonyl chloride 423 (738 mg, 3.33 mmol), and further stirred overnight at the room temperature. The solvents were removed and the residue was dissolved in CH₂Cl₂, washed with water and brine, dried with Na₂SO₄ and concentrated under reduced pressure. The material was purified by flash chromatography on silica gel eluting with Et₂O:MeOH (9:1) and further recrystallized from CH₂Cl₂:iPr₂O to give 2-(4-((4-nitro-1H-imidazole-1-yl)methyl)-1H-1,2,3-triazol-1-yl)-3-(((2-nitrophenyl)sulfonyl)oxy) propyl acetate 367 (547 mg, 40%) as a white foam. ¹HNMR [(CD₃)₂SO] δ 8.35 (d, J=1.4 Hz, 1H), 8.31 (br, s, 1H), 8.09-8.00 (m, 3H), 7.93-7.89 (m, 2H), 5.41 (s, 2H), 5.34-5.28 (m, 1H), 4.80-4.72 (m, 2H), 4.48-4.40 (m, 2H), 1.91 (s, 3H). HRMS (ESI) Calc. for C₁₇H₁₇N₇NaO₃S [M+Na]⁺ m/z 518.0710. found 5180701. HPLC Purity 95%.

### Example 2

### One-Electron Reduction Potential [E(1)] of Compounds 67 and 93

Electron-affinic nitroheterocyclic or nitroaromatic compounds can be selectively reduced by 1-electron processes in the hypoxic regions of solid tumours, in contrast to under normoxic conditions in normal tissues, to form a nitroso or hydroxylamine species that can covalently modify macromolecules and therefore be retained in hypoxic cells (Brown and Wilson, Nature Rev. Cancer, 2004, 4, 437-447). The nitroheterocyclic or nitroaromatic compounds should contain a nitro group possessing a 1-electron reduction potential, E(1), preferably between −0.45 V to −0.30V vs. NHE. The E(1) values of many compounds can be obtained from the literature, (for example, Wardman, P. J. Phys. Chem. Ref. Data, 1989, 18, 1637-1755.) or determined by a number of methods. The pulse radiolysis method, for example, measures the equilibrium constant between the radical anions of the nitroheterocyclic or nitroaromatic compound, formed upon their 1-electron reduction, and reference standards such as viologen and quinone compounds, from which data the E(1) values of the compounds can be calculated. (Meisel and Czapski. J. Phys. Chem., 1975, 79, 1503-1509.)

To confirm compounds 67 and 93 of the present invention possess 1-electron reduction potentials too low to have significant metabolism and retention in mammalian cells under hypoxia, relative to the known non-labelled hypoxia PET imaging agent EF5 (compound 15), a linear accelerator delivering short pulses of high energy electrons (2-3 Gy in 200 ns of 4 MeV) equipped with a fast spectophotometric detection system was used. (Anderson et al, J. Phys. Chem. A, 101, 9704-9709, 1997). Compounds were dissolved in N₂O-saturated solutions containing formate ions, as above, which, following pulse radiolysis, resulted in the rapid formation of the radical anions of the compounds within a few microseconds.

The E(1) values of compounds 15, 67 and 93 were measured by the pulse radiolysis method and while compound 15 was determined to be within the appropriate range for hypoxic metabolism in mammalian cells compounds 67 and 93 where shown to be significantly lower in electron affinity such that they fall outside the preferred range for hypoxic metabolism, binding and therefore retention in hypoxic cells (Table 1).

TABLE 1
Radiolytic reduction of selected compounds by the CO₂ ^{.-} radical.

| Compound | E(1)/V^{a} |
|---|---|
| 15 | −0.395 |
| 67 | −0.501 |
| 93 | −0.578 |

Footnotes for Table 1
^{a}Determined against methylviologen, E(1)MV²⁺/MV^{+.} = −447 ± 7 mV.

### Example 3

### A Bacterial Nitroreductase Library Over-Expressed in E. coli for Screening Bacterial Nitroreductase Metabolism of Nitroheterocyclic and Nitroaromatic Compounds

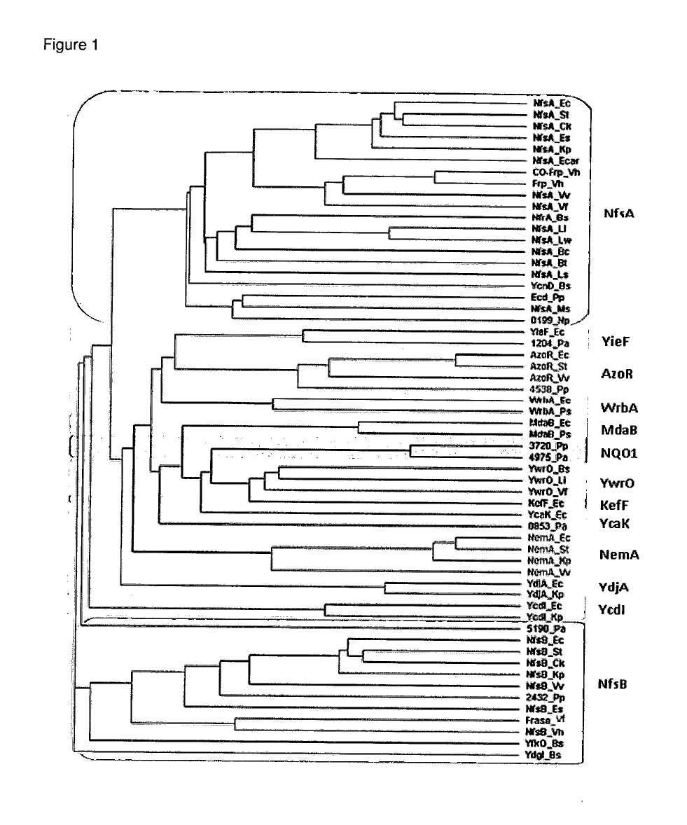

FIG. 1 illustrates the family relationships of the 58 nitroreductase (NTR) candidates in the E. coli NTR over-expression library, derived from 13 bacterial enzyme families. Multiple sequence alignment was performed using ClustalW2 (http://www.ebi.ac.uk/Tools/msa/clustalw2/). Grouping of enzymes into families was based on the degree of shared sequence identity with the closest E. coli representative for all families except for YwrO and NQO1, which (lacking clear E. coli orthologues) were aligned against Bacillus amyloliquefaciens YwrO or Homo sapiens NQO1, respectively. To distinguish genes or enzymes with the same family name, for the purpose of this work each NTR candidate was referred to using standard nomenclature followed by an underscore or parentheses enclosing a two letter abbreviation of the genus and species, e.g. NfsA_Kp and NemA_Ec or NfsA (K.p) and NemA (E.c) for the NfsA enzyme from K. pneumoniae and NemA enzyme from E. coli, respectively. The full list of candidate genes in the 58-membered NTR library is as follows, ordered alphabetically by the bacterial strain (underlined) that each was amplified from: Bacillus coagulans (strain 36D1) nfsA; Bacillus subtilis (ATCC 6051) nfrA, ycnD, ydgI, yfkO, ywrO; Bacillus thuringiensis serovar konkukian (strain 97-27) nfsA; Citrobacter koseri (ATCC 27156) nfsA, nfsB; Enterobacter (Chronobacter) sakazakii (ATCC 29544) nfsA, nfsB; Erwinia carotovora subsp. atrosepticum (strain SCRI1043) nfsA; Escherichia coli (W3110) azoR, kefF, mdaB, nemA, nfsA, nfsB, wrbA, ycaK, ycdI, ydjA, yieF; Klebsiella pneumoniae (ATCC 13883) nemA, nfsA, nfsB, ycdI, ydjA; Lactobacillus sakei subsp. sakei (strain 23K) nfsA; Listeria innocua (Clip11262) nfsA, ywrO; Listeria welshimeri serovar 6b (strain SLCC5334) nfsA; Mycobacterium smeqmatis (strain MC2 155) nfsA; Nostoc punctiforme (PCC 73102) nfsA; Pseudomonas aeruqinosa (PAO1) nfsB (PA5190), nqo1 (PA4975), ycaK (PA0853), yieF (PA 1204); Pseudomonas putida (KT2440) azoR (PP4538), nfsA (PP2490), nfsB (PP2432), nqo1 (PP3720); Pseudomonas syringae pv. phaseolicola (1448a) mdaB, wrbA; Salmonella typhi (ATCC 19430) azoR, nemA, nfsA, nfsB; Vibrio fischeri (ATCC 7744) FRaseI (flavin reductase 1), nfsA, ywrO; Vibrio harveyi (ATCC 33843) co-frp (flavin reductase P), nfsB; Vibrio harvevi (KCTC 2720) frp (flavin reductase P); Vibrio vulnificus (ATCC 27562) azoR, nfsA, nfsB, nemA. All strains were from existing Victoria University stocks or sourced from the Environmental Science and Research Ltd. bacterial strain collection (Porirua, New Zealand). The E. coli strain used for over-expression of all NTR candidate genes was SOS-R2, a nfsA nfsB nemA azoR to/C deletion mutant derived from E. coli strain SOS-R1 as described in [G A Prosser, J N Copp, S P Syddall, E M Williams, J B Smaill, W R Wilson, A V Patterson and D F Ackerley. 2010. Discovery and evaluation of Escherichia coli nitroreductases that activate the anti-cancer prodrug CB1954. Biochemical Pharmacology 79: 678-687].

### Example 4

### Bacterial Nitroreductase Metabolism Profiles of Compounds 67, 93 and 97

FIG. 2 illustrates the metabolism of compound 67 by members of the 58-membered NTR over-expression library as measured by (A) Growth Inhibition assay and (B) SOS assay. (A) Growth Inhibition assay. Turbidity (OD₆₀₀) of NTR over-expressing cell cultures was recorded directly before and after 4 h incubation with 400 μM compound 67. Percentage Growth Inhibition represents the decrease in OD₆₀₀ of challenged cells relative to unchallenged control cells for each strain post-incubation (i.e. 100−[100×OD₆₀₀ of challenged cells/OD₆₀₀ of unchallenged cells]). Data are the average of 2 independent assays and the error bars indicate ±1 standard deviation. Labelled bars indicate the NfsA and NfsB family members within the NTR library. (B) SOS assay. Compound 67 demonstrates an ability to evoke the E. coli SOS (DNA damage repair) response upon activation. The data presented is the SOS response, measured by β-galactosidase activity (in Miller units), of NTR over-expressing E. coli SOS-R2 after 4 h challenge with 8 μM compound 67. Full details of assay protocol and calculations are as described in G A Prosser, J N Copp, S P Syddall, E M Williams, J B Smaill, W R Wilson, A V Patterson and D F Ackerley. 2010. Discovery and evaluation of Escherichia coli nitroreductases that activate the anti-cancer prodrug CB1954. Biochemical Pharmacology 79: 678-687. Data are the average of 2 independent assays and the error bars indicate ±1 standard deviation. The dashed line indicates the baseline activity for the empty plasmid control, and labelled bars indicate the NfsA and NfsB family members within the NTR library.

FIG. 2.1 illustrates the metabolism of compound 93 by members of the 58-membered NTR over-expression library as measured by Growth Inhibition assay. The assay was performed as described for FIG. 2(A), above, except that challenged cultures were incubated with 130 μM compound 93. Data are the average of 2 independent assays and the error bars indicate ±1 standard deviation. Labelled bars indicate the NfsA and NfsB family members within the NTR library.

FIG. 2.2 illustrates the metabolism of compound 97 by members of the 58-membered NTR over-expression library as measured by Growth Inhibition assay. The assay was performed as described for FIG. 2(A), above, except that challenged cultures were incubated with 800 μM compound 97. Data are the average of 2 independent assays and the error bars indicate ±1 standard deviation. Labelled bars indicate the NfsA, NfsB and NemA family members within the NTR library.

NTR library screening indicates compound 67 is readily reductively metabolised at the nitro moiety by the majority of NfsA family members along with a subset of the NfsB family, to produce cytotoxic metabolites that either inhibit the growth of the NTR over-expressing bacteria or induce an SOS response in the NTR over-expressing bacteria. Compound 93 is selectively reductively metabolised at the nitro moiety by the NfsA family to produce cytotoxic metabolites that inhibit the growth of the NTR over-expressing bacteria. Compound 97 is reductively metabolised at the nitro moiety by the majority of the NfsA and NfsB families tested to produce cytotoxic metabolites that inhibit the growth of the NTR over-expressing bacteria. Modest metabolism of compound 97 is also observed for members of the NemA nitroreductase family.

### Example 4.1

### Initial Rates of Metabolism of Compounds 67, 93 and 97 by Purified Recombinant Bacterial Nitroreductase Enzymes in the Presence of NADPH Co-Factor

The relative initial rates of reductive metabolism of the nitro moiety of test compounds by bacterial nitroreductase's can be measured experimentally by incubating the test compound at near-saturating concentration (determined empirically) with the purified recombinant bacterial nitroreductase enzymes in the presence of NADPH co-factor. UV/Vis spectroscopy is used to measure consumption of the co-factor, indicating metabolism of the test compound by the nitroreductase.

Compounds 67, 93 and 97 (500 μM) were added to NADPH (200 μM) in 10 mM Tris-Cl pH 7.0. Reactions were initiated by enzyme addition (between 5 and 20 μg per reaction). Rates represent μmol of NADPH consumed per mg enzyme added per minute.

Results were consistent with the results of the NTR over-expression library assays (FIGS. 2, 2.1 and 2.2). Compounds 67, 93, and 97 were readily metabolised by the selected purified recombinant bacterial nitroreductase enzymes at rates ranging from 0.82 to 12.2 μmol/min/mg (Tables 2, 3 and 4).

TABLE 2
Rates of metabolism of compound 67 by selected NTRs

| Enzyme | Rate (μmol/min/mg) | Error (1 std. dev.) |
|---|---|---|
| CO-Frp (V.h) | 1.1 | 0.2 |
| NfrA (B.s) | 1.7 | 0.1 |
| NfsA (E.c) | 2.7 | 0.4 |
| NfsB (E.s) | 2.4 | 0.2 |

TABLE 3
Rates of metabolism of compound 93 by selected NTRs

| Enzyme | Rate (μmol/min/mg) | Error (1 std. dev.) |
|---|---|---|
| CO-Frp (V.h) | 12.2 | 3.1 |
| NfrA (B.s) | 7.5 | 1.3 |
| NfsA (E.c) | 7.6 | 1.6 |
| NfsB (E.c) | 0.82 | 0.11 |

TABLE 4
Rates of metabolism of compound 97 by selected NTRs

| Enzyme | Rate (μmol/min/mg) | Error (1 std. dev.) |
|---|---|---|
| YcnD (B.s) | 6.2 | 4.1 |
| NfrA (B.s) | 3.9 | 0.9 |
| NfsA (E.c) | 4.3 | 1.1 |
| NfsB (E.c) | 1.4 | 0.2 |

### Example 4.2

### 50% Inhibitory Concentration (IC₅₀) of Compounds 67, 93 and 97 in NTR Over-Expressing Bacteria for Selected NTR Library Strains

FIG. 2.3 illustrates the IC₅₀ of compound 67 for selected NTR library strains (i.e. the concentration of compound 67 that yielded only 50% turbidity relative to an unchallenged control 4 h post-challenge, in replicate cultures across a serial dilution of compound 67). The strains selected were all those observed to have SOS activity above the empty plasmid control (“Empty”) in response to challenge with compound 67 as illustrated in FIG. 2(B), plus NfsB (E.c) as a negative control. For the IC₅₀ assays, 100 μl of overnight cultures were used to inoculate 2 ml M63 minimal medium supplemented with 100 μg·ml⁻¹ ampicillin and 50 μM IPTG and incubated at 30° C., 200 rpm for 3.5 h. 40 μL aliquots from each culture were then added to individual wells of a sterile 384 well plate, each already containing 40 μl of ampicillin and IPTG-supplemented M63 medium and a dilution series of compound 67 as indicated on the X-axis of panel A. A. Raw growth curves. Each NTR over-expression strain was tested at least in duplicate (independent replicates), with the exception of NfsB (E.c), which was only measured once. Culture turbidity was monitored by optical density at 600 nm 4 h post-challenge. B. IC₅₀ values calculated by comparison of the challenged cells with the unchallenged control for each strain, after subtracting the initial absorbance values (t=0 h), using SigmaPlot 10.0 (Systat Software Inc., Richmond, Calif.). Errors are 1 standard error of the mean (SEM).

FIG. 2.4 illustrates the IC₅₀ of compound 93 for selected NTR library strains. IC₅₀ assays were performed exactly as described for FIG. 2.3, above. NfrA (B.s) and CO_Frp (V.h) were selected on the basis of being the two most active enzymes observed in Growth Inhibition assays (FIG. 2.1), and NfsA (E.c) and NfsB (E.c) as the standard benchmark NTRs. “Empty” refers to the empty plasmid control strain. A. Raw growth curves. Each NTR over-expression strain was tested in duplicate (independent replicates). B. IC₅₀ values calculated by comparison of the challenged cells with the unchallenged control for each strain, after subtracting the initial absorbance values (t=0 h), using SigmaPlot 10.0 (Systat Software Inc., Richmond, Calif.). Errors are 1 standard error of the mean (SEM).

FIG. 2.5 illustrates the IC₅₀ of compound 97 for selected NTR library strains. IC₅₀ assays were performed exactly as described for FIG. 2.3, above. NfrA (B.s) and YcnD (B.s) were selected on the basis of being the two most active enzymes observed in Growth Inhibition assays (FIG. 2.2), and NfsA (E.c) and NfsB (E.c) as the standard benchmark NTRs. “Empty” refers to the empty plasmid control strain. A. Raw growth curves. Each NTR over-expression strain was tested in duplicate (independent replicates). B. IC₅₀ values calculated by comparison of the challenged cells with the unchallenged control for each strain, after subtracting the initial absorbance values (t=0 h), using SigmaPlot 10.0 (Systat Software Inc., Richmond, Calif.). Errors are 1 standard error of the mean (SEM).

Inhibition of bacterial cell growth was selectively observed for compounds 67, 93 and 97 in bacterial nitroreductase over-expressing E. coli strains compared to appropriate controls, a result consistent with metabolism of the nitro moiety of the test compounds by the over-expressed nitroreductase to produce anti-proliferative or cytotoxic metabolites.

### Example 4.3

### 50% Inhibitory Concentration (IC₅₀) Values of Compounds 15, 93, 67, 19 and 97 in HCT116 Wild Type (WT) Cancer Cells and HCT116 Cells Overexpressing the Nitroreductase NfsA from E. coli.

Table 5 shows the 50% inhibitory concentration (IC₅₀) values of compounds 15, 93, 67, 19 and 97 in HCT116 wild type (WT) cancer cells and HCT116 cells overexpressing the nitroreductase NfsA from E. coli. Inhibition of cell proliferation is a surrogate endpoint for cellular metabolism, binding and retention and indicates that NfsA can activate these compounds in vitro in a low cell density assay. IC₅₀ values were determined as the concentration of prodrug required to inhibit cell growth by 50% of untreated controls following 4 hour drug exposure, with washing and regrowth for 5 days. WT:NfsA ratios were determined as the WT IC₅₀/NfsA IC₅₀.

TABLE 5
IC₅₀ values of compounds 15, 93, 67, 19 and 97
in HCT116 wild type (WT) and HCT116-NfsA cells.

| Compound | WT IC₅₀ (μM) | NfsA IC₅₀ (μM) | WT:NfsA |
|---|---|---|---|
| 15 | 2270 | 1 | 2270 |
| 93 | 1455 | 14.5 | 100 |
| 67 | 6313 | 19 | 332 |
| 19 | >12500 | 16 | >781 |
| 97 | >12500 | 178 | >70 |

### Example 5

### Metabolism and Retention of Compounds 15, 67 and 93 by the Bacterial Nitroreductase E. coli NfsA when Expressed in Mammalian Cells

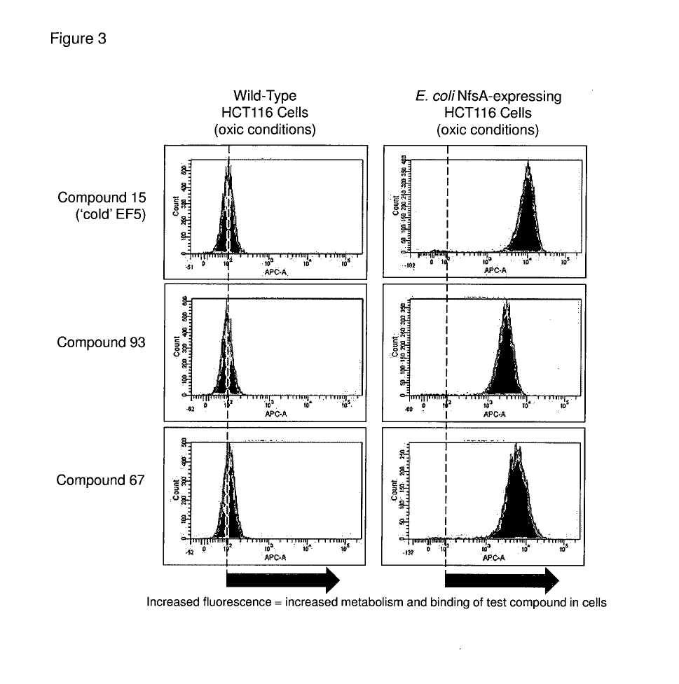

FIG. 3 illustrates the results of flow cytometry analysis of HCT-116 cells stably expressing E. coli NfsA relative to HCT-116 wild-type cells after in vitro exposure to 20 μM of compounds 15, 93 and 67 for 2 hours. 1×10⁶ cells were incubated with test compounds under oxic conditions. Samples were fixed and stained with EF5 antibody Alexa 488 ELK3.51 at 100 μg/ml. Samples were then analysed on a Becton Dickinson FACscan flow cytometer.

Compound 15, 67 and 93 are all excellent substrates for E. coli NfsA under oxic conditions (21% O₂, 5% CO₂) demonstrating evidence of metabolism and cellular retention in HCT-116 cells overexpressing E. coli NfsA by FACS analysis. In contrast minimal metabolism and binding is observed in wild-type HCT-116 cells with all test compounds demonstrating FACS profiles comparable to non-drug treated control cells.

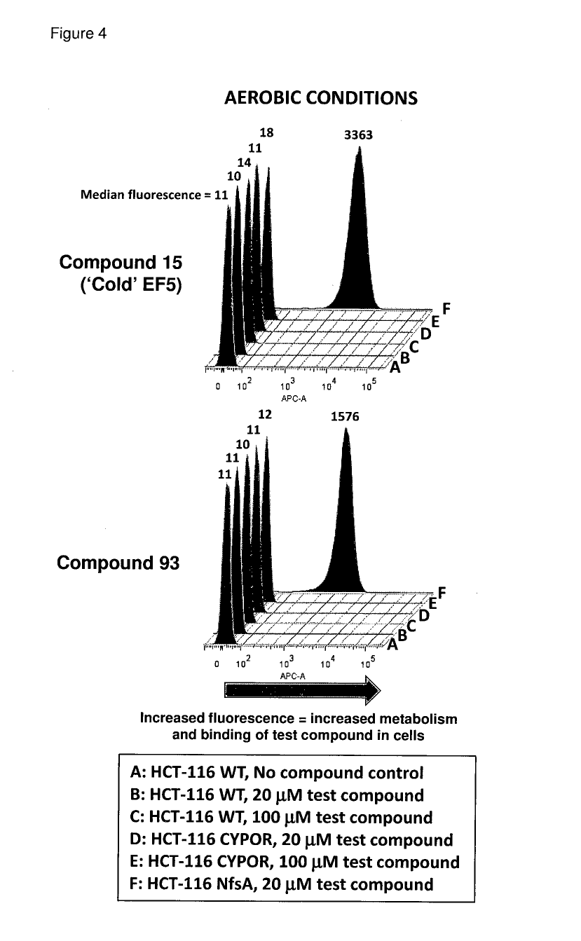

FIG. 4 illustrates the results of a second independent flow cytometry analysis of compound 15 and 93 metabolism and binding in wild-type HCT-116 cells, HCT-116 cells stably over-expressing cytochrome P450 reductase (CYPOR), a human one-electron reductase known to metabolise nitroheterocyclic and nitroaromatic compounds, or HCT-116 cells stably expressing the bacterial nitroreductase E. coli NfsA. 1×10⁶HCT-116 cells were seeded in 6 well plates underaerobic conditions (21% O₂, 5% CO₂). After 2 h incubation, drug free control (wild-type HCT-116, foreground), 20 μM (wild-type HCT-116, second plot), 100 μM (wild-type HCT-116, third plot), 20 μM (HCT-116-CYPOR, forth plot), 100 μM (HCT-116-CYPOR, fifth plot) or 20 μM (HCT-116-NfsA, sixth plot) of compound 15 or 93 was added. After 2 h incubation cells were harvested and fixed with paraformaldehyde before being incubated overnight with 100 μl of 75 μg/ml EF5 CY5 conjugated antibody. Samples were then analysed on a Becton Dickinson FACscan flow cytometer.

Compounds 15 and 93 are excellent substrates for E. coli NfsA under aerobic conditions demonstrating evidence of metabolism and cellular retention in HCT-116 cells overexpressing E. coli NfsA by FACS analysis. In contrast minimal metabolism and binding is observed in wild-type HCT-116 cells and HCT-116-CYPOR cells with both test compounds at both concentrations investigated, demonstrating FACS profiles comparable to non-drug treated control cells.

### Example 6

### Metabolism of Compound 67 Relative to ‘Cold’ EF5 (Compound 15) in HCT-116-CYPOR Cells Under Aerobic, Pathologically Hypoxic and Anoxic Conditions

FIG. 5 illustrates the results of flow cytometry analysis of HCT-116 cells stably over-expressing cytochrome P450 reductase (CYPOR), a human one-electron reductase known to metabolise nitroheterocyclic and nitroaromatic compounds. 1×10⁶HCT-116-CYPOR cells were seeded in 6 well plates in aerobic, anoxic and 0.2% oxygen conditions designed to replicate the lower limit of pathological hypoxia observed in human tumours. After 2 h incubation, drug free control (foreground), 20 μM (middle) or 100 μM (background) of compound 15 or 67 was added. After 2 h incubation cells were harvested and fixed with paraformaldehyde before being incubated overnight with 100 μl of 75 μg/ml EF5 CY5 conjugated antibody. Samples were then analysed on a Becton Dickinson FAC scan flow cytometer.

The known hypoxia imaging agent EF5 (compound 15) demonstrated negligible metabolism and binding in aerobic HCT-116-CYPOR cells. Significant dose-dependent increases in metabolism were observed in cells under 0.2% oxygen and anoxia respectively. In contrast, compound 67 showed negligible metabolism and binding in aerobic HCT-116-CYPOR cells and cells under 0.2% oxygen, indicating compound 67 is incapable of imaging human tumour hypoxia. Under severe anoxia compound 67 demonstrates 8 to 13-fold less retention in HCT-116-CYPOR cells than compound 15.

### Example 7

### Metabolism of Compound 93 Relative to ‘Cold’ EF5 (Compound 15) in HCT-116 Cells Under Anoxic Conditions

FIG. 6 illustrates the results of flow cytometry analysis of compound 15 and 93 metabolism and binding in wild-type HCT-116 cells and HCT-116 cells stably over-expressing cytochrome P450 reductase (CYPOR), a human one-electron reductase known to metabolise nitroheterocyclic and nitroaromatic compounds. 1×10⁶ HCT-116 cells were seeded in 6 well plates underanoxic conditions. After 2 h incubation, drug free control (wild-type HCT-116, foreground), 20 μM (wild-type HCT-116, second plot), 100 μM (wild-type HCT-116, third plot), 20 μM (HCT-116-CYPOR, forth plot) or 100 μM (HCT-116-CYPOR, fifth plot) of compound 15 or 93 was added. After 2 h incubation cells were harvested and fixed with paraformaldehyde before being incubated overnight with 100 μl of 75 μg/ml EF5 CY5 conjugated antibody. Samples were then analysed on a Becton Dickinson FACscan flow cytometer.

The known hypoxia imaging agent EF5 (compound 15) demonstrated significant dose-dependent and reductase dependent increases in metabolism and binding in HCT-116 cells under anoxia. In contrast, compound 93 showed negligible metabolism and binding in wild-type HCT-116 cells and HCT-116-CYPOR cells under anoxia indicating compound 93 is incapable of imaging human tumour hypoxia.

### Example 8

### Immunohistochemical Detection of the 2-Nitroimidazoles EF5 (Compound 15) and Pimonidazole Binding in Human HCT-116 and H1299 Xenografts Relative to Compounds 67 and 93

FIG. 7 illustrates immunohistochemical detection of ‘cold’ EF5 (compound 15) binding in human tumour xenografts harbouring 0% or 25% HCT-116 NfsA-expressing cells.

The mixed tumour xenograft expressing 25% of E. coli NfsA expressing HCT-116 cells results in significantly enhanced EF5 metabolism, binding and retention. However, a background signal of EF5 binding can be observed in HCT-116 wild-type xenografts consistent with metabolism and binding of EF5 in the hypoxic regions of the tumour. This background provides unwanted noise when seeking to determine the extent of introduced NTR expressing cells and/or biological agents. The present invention provides nitroheterocyclic and nitroaromatic compounds for PET imaging of NTR-expressing cells free of this background of tumour hypoxia.

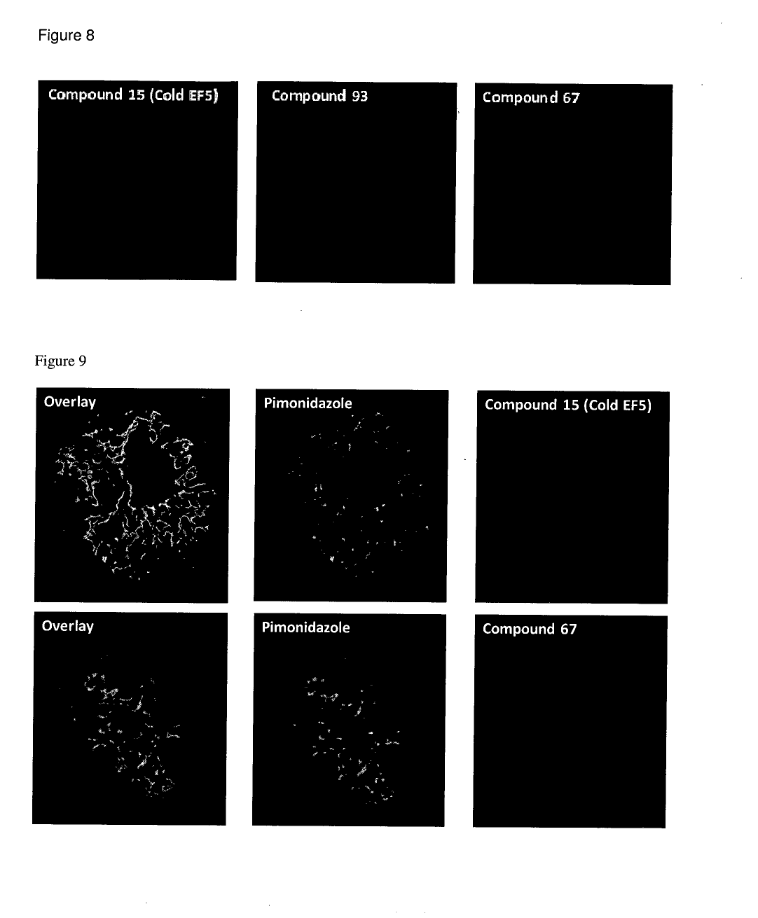

FIG. 8 illustrates the in vivo binding of compounds 15, 93 and 67 in the human lung tumour xenograft NCI-H1299 harbouring approximately 5% NfsA-positive cells. NfsA expressing cells are readily detected by immunohistochemistry with single cell resolution following binding of compound 15, compound 93 or compound 67. Mixed NfsA/WT NCI-H1299 cells were inoculated subcutaneously onto the flank of NIH-III nude mice. When the mixed tumours reached approximately 500 mm³, mice were dosed i.p. with 60 mg/kg of either compound 15, compound 93 or compound 67. After 60 minutes the tumours were excised, and fixed in formalin before being embedded in paraffin wax. Tumour section were cut (5 microns) and mounted onto glass slides for immunodetection of bound adducts of compound 15, compound 93 or compound 67 using the monoclonal antibody ELK3-51 directly conjugated to the fluorophore CY5 (Ex/Em 650/670 nm). Fluorescent microscopy was employed to visualise the presence of cellular adducts of each test compound present in individual tumour cells. Image gain was reduced due to intense fluorescent signal indicating extensive adduct binding. Images were acquired on a Zeiss LSM 710 confocal microscope (×20 magnification).

FIG. 9 illustrates the absence of hypoxic dependent binding of compound 67 in the human solid tumour xenograft HCT116 relative to compound 15 whilst including hypoxia co-staining by pimonidazole (Hypoxyprobe™) as an internal reference (positive control). HCT116 WT tumours were inoculated subcutaneously onto the flank of NIH-Ill nude mice. When the mixed tumours reached approximately 500 mm³, mice were dosed i.p. with 60 mg/kg of pimonidazole, and 60 minutes later dosed with either 60 mg/kg of compound 15 or 60 mg/kg of compound 67. After 120 minutes the tumours were excised, and fixed in formalin before being embedded in paraffin wax. Tumour section were cut (5 microns) and mounted onto glass slides for immunodetection of bound adducts of compounds. Immunofluorescent microscopy was performed using a monoclonal antibody (Mab1, hybridoma clone 4.3.11.3) conjugated to Alexa-488 (green) for the detection of pimonidazole adducts, and monoclonal antibody ELK3-51 directly conjugated to the fluorophore CY5 (Ex/Em 650/670 nm) for detection of adducts formed by compound 15 or compound 67. The overlap of pimonidazole with either one of these markers appears as yellow. Images were acquired on a Zeiss LSM 710 confocal microscope (×20 magnification). It is readily evident that compound 15 detects an identical set of hypoxic tumour cells as seen by pimonidazole, whereas compound 67 is not detected in the pimonidazole positive hypoxic regions of the tumour indicating the hypoxia-dependent binding and retention of compound 67 is absent.

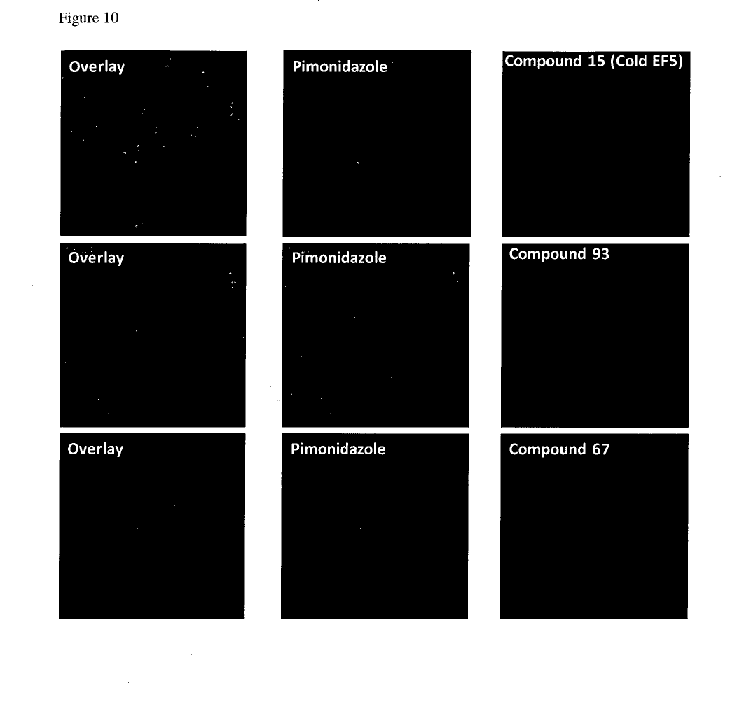

FIG. 10 illustrates the absence of hypoxic-dependent binding of compound 67 and compound 93 by fluorescent immune-histochemistry in the human solid tumour xenograft NCI-H1299, with reference to hypoxia staining by compound 15 and pimonidazole (Hypoxyprobe™) as internal standards (positive controls). NCI-H1299 WT tumours were inoculated subcutaneously onto the flank of NIH-Ill nude mice. When the mixed tumours reached approximately 800 mm³, mice were dosed i.p. with 60 mg/kg of pimonidazole, and 60 minutes later dosed with either 60 mg/kg of compound 15, or 60 mg/kg of compound 67, or 60 mg/kg of compound 93. After 120 minutes the tumours were excised, and fixed in formalin before being embedded in paraffin wax. Tumour section were cut (5 microns) and mounted onto glass slides for immunodetection of bound adducts of compounds. Immunofluorescent microscopy was performed using a monoclonal antibody (Mab1, hybridoma clone 4.3.11.3) conjugated to Alexa-488 (Ex/Em 499/519; green) for the detection of pimonidazole adducts, and monoclonal antibody ELK3-51 directly conjugated to the fluorophore CY5 (Ex/Em 650/670 nm; red) for detection of adducts formed by compound 15 or compound 67 or compound 93. The overlap of pimonidazole with any one of these markers appears as yellow. Images were acquired on a Zeiss LSM 710 confocal microscope (×20 magnification). It is readily evident that compound 15 detects an identical set of hypoxic tumour cells as that seen by pimonidazole in NCI-H1299 tumours, whereas compound 67 and compound 93 are not detected in the pimonidazole positive hypoxic regions of the NCI-H1299 tumours indicating the hypoxia-dependent binding and retention of compound 67 and compound 93 is absent.

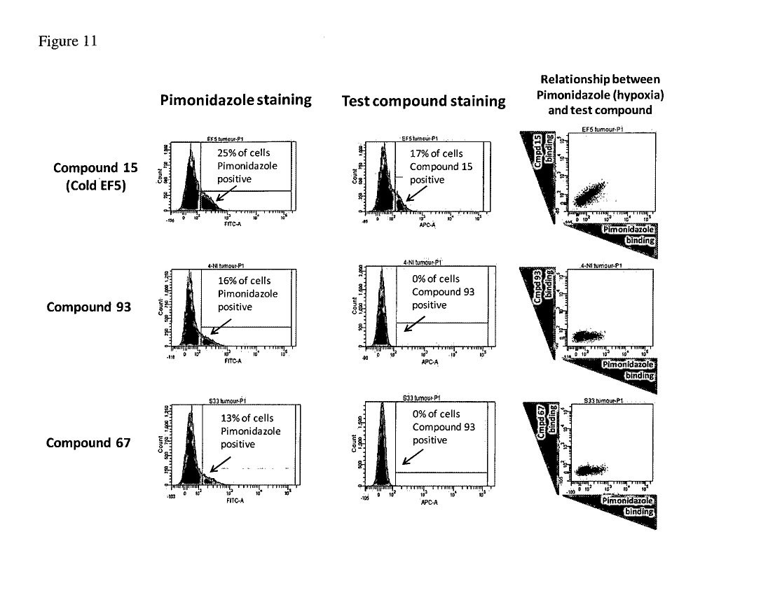

FIG. 11 illustrates the absence of hypoxic-dependent binding of compound 67 and compound 93 by flow cytometry in the human solid tumour xenograft NCI-H1299, with reference to hypoxia staining by compound 15 and pimonidazole (Hypoxyprobe™) as internal standards (positive controls). NCI-H1299 WT tumours were inoculated subcutaneously onto the flank of NIH-III nude mice. When the mixed tumours reached approximately 800 mm³, mice were dosed i.p. with 60 mg/kg of pimonidazole, and 60 minutes later dosed with either 60 mg/kg of compound 15, or 60 mg/kg of compound 67, or 60 mg/kg of compound 93. After 120 minutes the tumours were excised and enzyme digested to form a single cell suspension before fixation in ice cold 80% ethanol. Single cell immunodetection of bound adducts of compounds was performed using a monoclonal antibody (Mab1, hybridoma clone 4.3.11.3) conjugated to Alexa-488 (Ex/Em 499/519; green) for the detection of pimonidazole adducts, and monoclonal antibody ELK3-51 directly conjugated to the fluorophore CY5 (Ex/Em 650/670 nm; red) for detection of adducts formed by compound 15 or compound 67 or compound 93. The ex-vivo tumour cell samples were analysed on a Becton Dickinson FACscan flow cytometer using FACS Diva software. Integrated fluorescence measurements were recorded for 10,000 single non-debris events. Fluorescence emission was monitored at 530 nm±20 and 670-700 nm for detection of Alexa-488 and CY5 conjugated monoclonal antibodies, respectively. The left hand column of histograms labelled “Pimonidazole staining” illustrates that all three tumour samples contain hypoxic cells that are detected by pimonidazole adduct retention. The central column of histograms labelled “Test compound staining” indicates that compound 15 but not compounds 93 or 67 will bind and thus detect these hypoxic tumour cells. The right hand side column labelled “Relationship between pimonidazole (hypoxia) and test compound” is a series of dot plots that demonstrates that pimonidazole and compound 15 both detect the identical tumour cell population whereas compound 93 and 67 are unable to bind to and thus detect pimonidazole-positive (hypoxic) tumour cells. This demonstrates that compound 93 and compound 67 are free of undesirable hypoxic metabolism and retention in the human tumour xenograft NCI-H1299.

1. A method of imaging and/or ablation of a bacterial nitroreductase-expressing cell and/or a bacterial nitroreductase-expressing biological agent comprising:
i. introduction of a compound of formula I to a subject; and
ii. metabolising the compound with a bacterial nitroreductase expressed by the cell and/or biological agent;
wherein the compound is substantially insensitive to metabolism under oxic or hypoxic conditions in a cell or biological agent that does not express a bacterial nitroreductase; and
wherein formula I comprises: 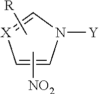
wherein:
a. when X═O, S or C—H,
R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
NO₂ is attached at any unsubstituted position; and
Y comprises a formula selected from the group consisting of formulae IIa to IIg: 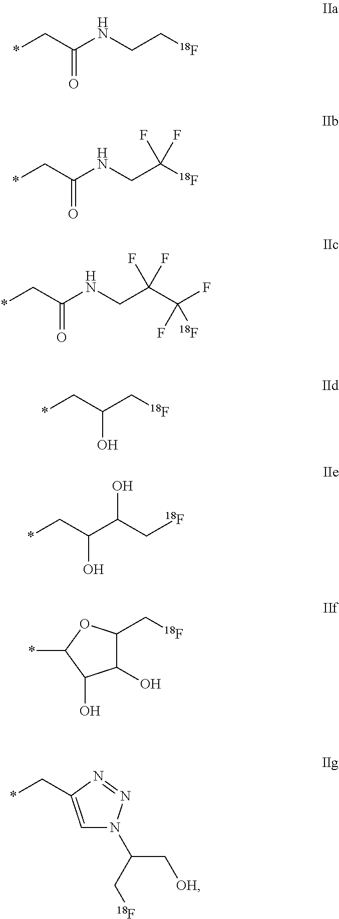
and IIIa to IIIh; 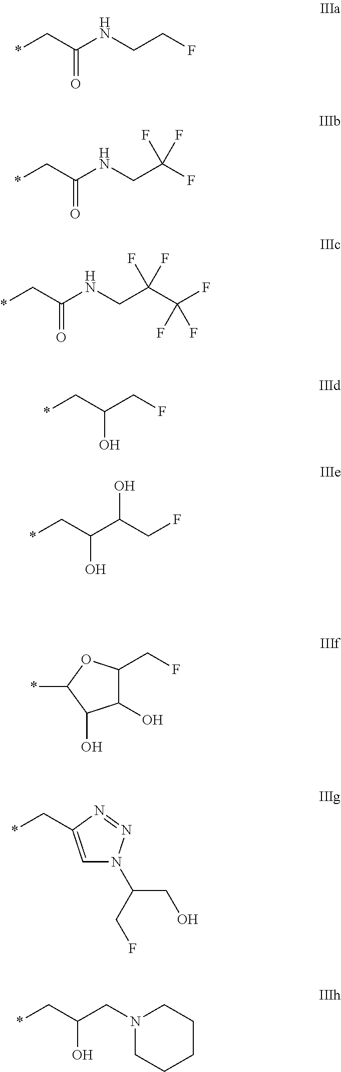
where *=a point of attachment to Formula I; or
b. when X═N,
R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
NO₂ is attached at the 4- or 5-position; and
Y is selected from the group consisting of formulae IIa-g and IIIa-h where *=a point of attachment to Formula I.

2. (canceled)

3. (canceled)

4. The method as claimed in claim 1, wherein the method comprises a method of immunohistochemical imaging and wherein Y is selected from IIIb, IIIc or IIIh and R is selected from CH₂F or CH₂¹⁸F.

5. (canceled)

6. A compound of formula I: 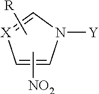
wherein:
a. when X═O, S or C—H,
R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
NO₂ is attached at any unsubstituted position; and
Y comprises a formula selected from the group consisting of formulae IIa to IIg and IIIa to IIIh as defined in claim 0 where *=a point of attachment to Formula I;
or a precursor thereof; or
b. when X═N,
R═H, CF₃, CH₂F, CH₂¹⁸F, OCF₃, SO₂C₁-C₆ alkyl, SOC₁-C₆ alkyl, CN, CONH₂, CONHC₁-C₆ alkyl, CON(C₁-C₆ alkyl)₂, OC₁-C₆ alkyl, C₁-C₆ alkyl;
NO₂ is attached at the 4- or 5-position; and
Y is selected from the group consisting of: formulae IIa-g and IIIa-IIc and IIIe to IIIh where *=a point of attachment to Formula I;
or a precursor thereof.

7. The compound according to claim 6 wherein the compound is a precursor compound and Y is selected from the group consisting of formulae IVa-g: 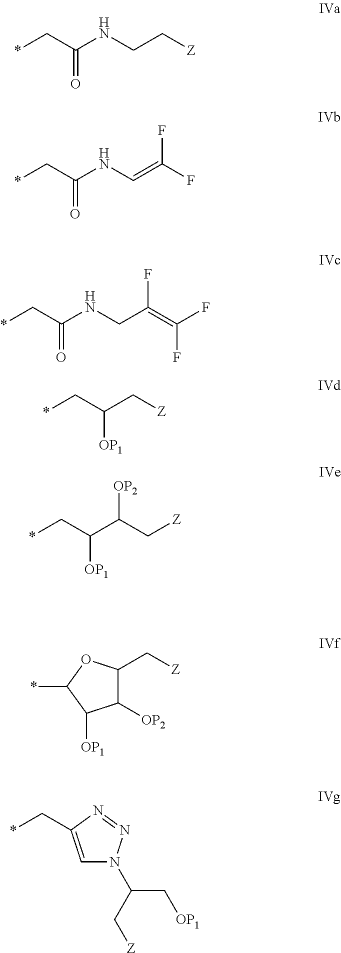
where *=a point of attachment to Formula I and Z═Cl, Br, I, OSO₂CH₃, OTs, ONs, OSO₂CF₃ and P₁ and P₂ can be independently selected from H, CO(C₁-C₆ alkyl), CO^{t}Bu, Si(CH₃)₃, Si(CH₃)₂^{t}Bu, Si(Ph)₂^{t}Bu, CH₂Ph, CH₂C₆H₄OMe, C(Ph)₃ or together may form an acetonide ring.

8. The compound according to claim 6 comprising a radiolabelled compound according to formula 104: 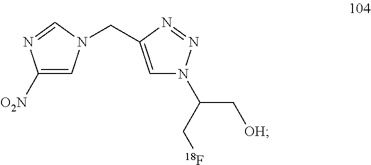 [SMILES: O=[N+]([O-])c1cn(Cc2cn(C(CO)C[18F])nn2)cn1]
or
a precursor compound according to formula 367: 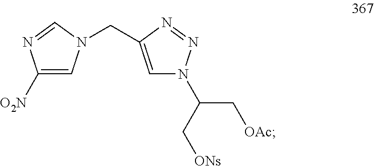
or
a compound according to formula 97:

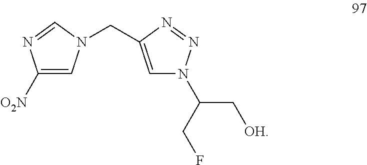 [SMILES: O=[N+]([O-])c1cn(Cc2cn(C(CO)CF)nn2)cn1]

9-21. (canceled)

22. A method of treatment or diagnosis of a disease using the compound of claim 6, wherein the disease is selected from the group consisting of cancer, Parkinson's disease, Alzheimer's disease, stroke, heart disease, rheumatological diseases and a disease treated by stem-cell transplantation.

23-31. (canceled)
